(19) United States
(12) Patent Application Publication
(10) Pub. No.: US 2015/0335665 A1
(43) Pub. Date: Nov. 26, 2015

(54) USE OF ETHANOLAMINE PHOSPHATE

(71) Applicant: KABUSHIKI KAISHA TOYOTA CHUO KENKYUSHO, Nagakute-shi (JP)

(72) Inventor: Minoru HIRANO, Nagakute-shi (JP)

(73) Assignee: KABUSHIKI KAISHA TOYOTA CHUO KENKYUSHO, Nagakute-shi (JP)

(21) Appl. No.: 14/815,431

(22) Filed: Jul. 31, 2015

Related U.S. Application Data
(63) Continuation of application No. 14/419,051, filed on Feb. 2, 2015.

(30) Foreign Application Priority Data
Jul. 31, 2012 (JP) 2012-169322
Jul. 29, 2013 (JP) 2013-156760
Jul. 30, 2013 (JP) 2013-158259

(51) Int. Cl. A61K 31/661

(52) U.S. Cl. CPC A61K 31/661

(57) ABSTRACT

A method of inhibiting oxidative stress to a cell that includes contacting a cell with an effective amount of ethanolamine phosphate. An animal cell function enhancer containing ethanolamine phosphate (EP) as an active ingredient. The presence of the ethanolamine phosphate enhances the function of the animal cell even in the absence of serum and growth factors.

This is a continuation of application Ser. No. 14/419,051 filed Feb. 2, 2015, which is a National Stage Application of PCT/JP2013/070815 filed Jul. 31, 2013, and claims the benefit of Japanese Application Nos. 2012-169322 filed Jul. 31, 2012, 2013-156760 filed Jul. 29, 2013, and 2013-158259 filed Jul. 30, 2013. The entire disclosures of the prior applications are hereby incorporated by reference in their entirety.

### TECHNICAL FIELD

This application claims priority based on Japanese Patent Application No. 2012-169322 filed on Jul. 31, 2012, Japanese Patent Application No. 2013-156760 filed on Jul. 29, 2013 and Japanese Patent Application No. 2013-158259, filed on Jul. 30, 2013, the entire contents of which are hereby incorporated by reference into the present specification.

The present specification relates to a use of ethanolamine phosphate.

### DESCRIPTION OF RELATED ART

Ethanolamine phosphate (which may be called simply ‘EP’ below) is widely distributed along with ethanolamine and ethanolamine phosphoglycerol in the living membranes and the like of animals. EP is also known as a precursor of phospholipids essential for cells, such as phosphatidylcholine (lecithin), which is a key constituent of cell membranes, and phosphatidylethanolamine, which is common in neural tissue.

Moreover, it is known that EP and ethanolamine can be added to media as enhancers of cell growth factors (Japanese Patent Application Publication No. H6-78759, Japanese Patent Application Publication No. H4-501660, and Japanese Patent Application Publication No. 2000-506374). EP is also an active component that enhances acetaldehyde dehydrogenase activity, and is known to be useful for hangovers and other liver disorders and disorders of the intestinal mucosa (Japanese Patent Application Publication No. 2002-104961, Japanese Patent Application Publication No. H8-310994, and International Publication No. WO 99/39703). It has also been reported to be a potential biomarker for depression.

### BRIEF SUMMARY

The actions of EP are diverse as described above. The present specification provides a novel use of EP. Not all of the actions of EP have been elucidated. It is entirely unknown whether EP promotes or suppresses the expressions of any genes when it acts on cells. If the genes associated with the onset of action of EP could be elucidated, it would be possible to discover useful functions of EP, provide novel uses of EP, and search for compounds that enhance the action of EP or act on cells in the same way as EP.

The inventors performed various studies focusing on the actions of EP on cells. As a result, it was discovered that EP has new functions not available in the past, and that novel uses could be provided based on these functions. The inventors have also clarified the expression levels of genes associated with the actions of EP, and have further elucidated the actions of EP. The present specification provides a novel use of EP based on these findings.

The inventors discovered new actions of EP on cells when EP is administered to cells, and also analyzed genes whose expression is promoted or suppressed by EP. Promotion of the expression of a specific gene group and suppression of the expression of a specific gene group were also confirmed through gene expression analysis. New actions were also discovered. The following means are provided based on these findings.

(1) An animal cell function enhancer containing ethanolamine phosphate as an active ingredient.

(2) The animal cell function enhancer according to (1), wherein the enhancer is a cell proliferation agent in an absence of serum and growth factors.

(3) The animal cell function enhancer according to (1) or (2), wherein the enhancer is a cell proliferation agent for one or two or more kinds of animal cells selected from the group consisting of airway epithelial cells, lung fibroblasts, skin epidermal cells and hair dermal papilla cells.

(4) The animal cell function enhancer according to any of (1) to (3), wherein the enhancer is a cell proliferation agent that selectively promotes proliferation of normal cells more than proliferation of atypical cells.

(5) The animal cell function enhancer according to (1), wherein the enhancer is a promoter of a barrier function of epithelial system cells.

(6) The animal cell function enhancer according to (1), wherein the enhancer is an apoptosis suppressor.

(7) The animal cell function enhancer according to (1), wherein the enhancer is a cultured cell structure preparation agent.

(8) A hair growth agent comprising ethanolamine phosphate an active ingredient.

(9) A method for enhancing a function of animal cells by bringing the animal cells into contact with ethanolamine phosphate outside an animal body.

(10) A method for screening agents that act on animal cells, comprising step of:

bringing the animal cells into contact with a test compound in a presence of ethanolamine phosphate, and measuring effects on the animal cells.

The following means are also provided:

(1) An inflammatory cytokine suppressor comprising ethanolamine phosphate as an active ingredient.

(2) An anti-inflammatory agent comprising ethanolamine phosphate as an active ingredient.

(3) An antioxidant having ethanolamine phosphate as an active ingredient.

(4) A composition for prevention or treatment of inflammatory disease, wherein the composition comprises ethanolamine phosphate as an active ingredient.

(5) A gene expression method, wherein expression of one or more genes selected from a first gene group shown below is promoted and expression of one or more genes selected from a second gene group shown below is suppressed by administering ethanolamine phosphate to animal cells:

### (First Gene Group)

TABLE 1

| ID | GeneSymbol | Description | GenbankAccession | log2 [ratio] | R* | p-value |
|---|---|---|---|---|---|---|
| A_23_P250554 | AGGF1 | a1 | NM_018046 | 0.607 | 1.523 | 0.012 |
| A_23_P146644 | ANXA2 | a2 | NM_001002857 | 0.742 | 1.673 | 0.007 |
| A_32_P94798 | ANXA2 | a3 | NM_001002857 | 0.691 | 1.614 | 0.018 |
| A_23_P110151 | ARHGAP24 | a4 | NM_001025616 | 0.740 | 1.670 | 0.035 |
| A_24_P388659 | ARHGAP24 | a5 | NM_001025616 | 0.803 | 1.745 | 0.011 |
| A_23_P54144 | BMP4 | a6 | NM_001202 | 0.864 | 1.820 | 0.003 |
| A_23_P55544 | CCBE1 | a7 | NM_133459 | 0.811 | 1.755 | 0.005 |
| A_32_P171043 | CCBE1 | a8 | NM_133459 | 0.818 | 1.890 | 0.006 |
| A_23_P211762 | COL8A1 | a9 | CN371015 | 0.716 | 1.643 | 0.035 |
| A_23_P69030 | COL8A1 | a10 | NM_001850 | 0.999 | 1.999 | 0.005 |
| A_24_P139152 | COL8A1 | a11 | AL359062 | 0.768 | 1.703 | 0.017 |
| A_23_P105803 | FGF9 | a12 | NM_002010 | 0.684 | 1.606 | 0.008 |
| A_23_P373521 | HAND2 | a13 | NM_021973 | 0.979 | 1.971 | 0.028 |
| Description | | | | | | |
| a1 | Homo sapiens angiogenic factor with G patch and FHA domains 1 (AGGF1), | | | | | |
| | mRNA [NM 018046] | | | | | |
| a2 | Homo sapiens annexin A2 (ANXA2), transcript variant 2, mRNA [NM 001002857] | | | | | |
| a3 | Homo sapiens annexin A2 (ANXA2), transcript variant 2, mRNA [NM 001002857] | | | | | |
| a4 | Homo sapiens Rho GTPase activating protein 24 (ARHGAP24), transcript variant 1, | | | | | |
| | mRNA [NM 001025616] | | | | | |
| a5 | Homo sapiens Rho GTPase activating protein 24 (ARHGAP24), transcript variant 1, | | | | | |
| | mRNA [NM 001025616] | | | | | |
| a6 | Homo sapiens bone morphogenetic protein 4 (BMP4), transcript variant 1, mRNA | | | | | |
| | [NM 001202] | | | | | |
| a7 | Homo sapiens collagen and calcium binding EGF domains 1 (CCBE1), mRNA | | | | | |
| | [NM 133459] | | | | | |
| a8 | Homo sapiens collagen and calcium binding EGF domains 1 (CCBE1), mRNA | | | | | |
| | [NM 133459] | | | | | |
| a9 | collagen, type VIII, alpha 1 [Source: HGNC Symbol; Acc: 2215] | | | | | |
| | [ENST00000261037] | | | | | |
| a10 | Homo sapiens collagen, type VIII, alpha 1 (COL8A1), transcript variant 1, mRNA | | | | | |
| | [NM 001850] | | | | | |
| a11 | collagen, type VIII, alpha 1 [Source: HGNC Symbol; Acc: 2215] | | | | | |
| | [ENST00000261037] | | | | | |
| a12 | Homo sapiens fibroblast growth factor 9 (glia-activating factor) (FGF9), mRNA | | | | | |
| | [NM 002010] | | | | | |
| a13 | Homo sapiens heart and neural crest derivatives expressed 2 (HAND2), mRNA | | | | | |
| | [NM 021973] | | | | | |
| R*: | Ratio to control (times) | | | | | |
| ID | GeneSymbol | Description | GenbankAccession | log2 [ratio] | R* | p-value |
| A_24_P231104 | LEPR | a14 | NM_001003679 | 0.730 | 1.659 | 0.003 |
| A_23_P122216 | LOX | a15 | NM_002317 | 0.711 | 1.637 | 0.001 |
| A_23_P163787 | MMP2 | a16 | NM_004530 | 0.658 | 1.578 | 0.002 |
| A_23_P88589 | NR2F2 | a17 | NM_021005 | 0.733 | 1.662 | 0.005 |
| A_24_P313354 | NR2F2 | a18 | NM_021005 | 0.804 | 1.746 | 0.001 |
| A_24_P135322 | NRP1 | a19 | NM_001024629 | 0.735 | 1.664 | 0.003 |
| A_24_P928052 | NRP1 | a20 | NM_003873 | 0.993 | 1.990 | 0.001 |
| A_23_P201808 | PPAP2B | a21 | NM_003713 | 0.941 | 1.919 | 0.006 |
| A_23_P104881 | ROBO4 | a22 | NM_019055 | 0.836 | 1.785 | 0.011 |
| A_23_P344421 | ROBO4 | a23 | NM_019055 | 0.707 | 1.633 | 0.005 |
| A_23_P56933 | RTN4 | a24 | NM_020532 | 0.594 | 1.510 | 0.014 |
| A_23_P211957 | TGFBR2 | a25 | NM_001024847 | 0.708 | 1.634 | 0.009 |
| A_23_P337242 | TGFBR2 | a26 | NM_001024847 | 0.705 | 1.630 | 0.005 |
| A_23_P167096 | VEGFC | a27 | NM_005429 | 0.711 | 1.637 | 0.005 |
| A_23_P74380 | WARS2 | a28 | NM_201263 | 0.673 | 1.594 | 0.036 |
| A_23_P168909 | ZFPM2 | a29 | NM_012082 | 0.746 | 1.678 | 0.018 |

Description

| | |
|---|---|
| a14 | Homo sapiens leptin receptor (LEPR), transcript variant 3, mRNA |
| | [NM 001003679] |
| a15 | Homo sapiens lysyl oxidase (LOX), mRNA [NM 002317] |
| a16 | Homo sapiens matrix metallopeptidase 2 (gelatinase A, 72 kDa gelatinase, 72 kDa |
| | type IV collagenase) (MMP2), transcript variant 1 mRNA [NM 004530] |
| a17 | Homo sapiens nuclear receptor subfamily 2, group F, member 2 (NR2F2), |
| | transcript variant 1, mRNA [NM 021005] |
| a18 | Homo sapiens nuclear receptor subfamily 2, group F, member 2 (NR2F2), |
| | transcript variant 1, mRNA [NM 021005] |
| a19 | Home sapiens neuropilin 1 (NRP1), transcript variant 3, mRNA [NM 001024629] |
| a20 | Homo sapiens neuropilin 1 (NRP1), transcript variant 1, mRNA [NM 003673] |
| a21 | Homo sapiens phosphatidic acid phosphatase type 2B (PPAP2B), mRNA |
| | [NM 003713] |
| a22 | Homo sapiens roundabout homolog 4, magic roundabout (Drosophila) (ROBO4), |
| | mRNA [NM 019055] |
| a23 | Homo sapiens roundabout homolog 4, magic roundabout (Drosophila) (ROBO4), |
| | mRNA [NM 019055] |
| R*: | Ratio to control (times) |
| a24 | Homo sapiens reticulon 4 (RTN4), transcript variant 1, mRNA [NM 020532] |
| a25 | Homo sapiens transforming growth factor, beta receptor II (70/80 kDa) |
| | (TGFBR2), transcript variant 1, mRNA [NM 001024847] |
| a26 | Homo sapiens transforming growth factor, beta receptor II (70/80kDa) |
| | (TGFBR2), transcript variant 1, mRNA [NM 001024847] |
| a27 | Homo sapiens vascular endothelial growth factor C (VEGFC), mRNA [NM 005429] |
| a28 | Home sapiens tryptophanyl tRNA synthetase 2, mitochondrial (WARS2), nuclear |
| | gene encoding mitochondrial protein, transcript variant 2, mRNA [NM 201263] |
| a29 | Homo sapiens zinc finger protein, multitype 2 (ZFPM2), mRNA [NM 012082] |
| R*: | Ratio to control (times) |

TABLE 2

| ID | GeneSymbol | Description | GenbankAccession | log2 [ratio] | R* | p-value |
|---|---|---|---|---|---|---|
| A_24_P302833 | APPL1 | b1 | NM_012096 | 0.785 | 1.723 | 0.005 |
| A_24_P935986 | BCAT1 | b2 | NM_005504 | 0.783 | 1.721 | 0.001 |
| A_23_P118061 | CKLF | b3 | NM_181641 | 0.586 | 1.501 | 0.040 |
| A_23_P206396 | CKLF | b4 | NM_001040138 | 0.679 | 1.601 | 0.006 |
| A_24_P215804 | CKLF | b5 | NM_016951 | 0.633 | 1.551 | 0.044 |
| A_23_P71727 | CKS2 | b6 | NM_001827 | 0.595 | 1.510 | 0.003 |
| A_23_P500034 | GLI2 | b7 | NM_005270 | 1.043 | 2.060 | 0.007 |
| A_24_P193911 | MAPRE2 | b8 | NM_014268 | 0.736 | 1.666 | 0.002 |
| A_23_P89835 | MAPRE2 | b9 | CR597075 | 0.621 | 1.538 | 0.023 |
| A_24_P38363 | MYC | b10 | NM_002467 | 0.754 | 1.686 | 0.025 |
| A_24_P313504 | PLK1 | b11 | NM_005030 | 0.955 | 1.939 | 0.004 |
| A_23_P118174 | PLK1 | b12 | NM_005030 | 1.291 | 2.447 | 0.000 |
| A_23_P156310 | SKP2 | b13 | NM_032637 | 0.615 | 1.531 | 0.001 |
| A_23_P149529 | TACSTD2 | b14 | NM_002353 | 0.591 | 1.507 | 0.003 |
| A_23_P37736 | TNFRSF17 | b15 | NM_001192 | 1.336 | 2.525 | 0.005 |
| A_23_P28625 | WDR12 | b16 | NM_018256 | 0.745 | 1.676 | 0.004 |

| | | |
|---|---|---|
| Description | b1 | Homo sapiens adaptor protein, phosphotyrosine interaction, PH domain and leucine zipper |
| | | containing 1 (APPL1), mRNA [NM_012096] |
| | b2 | Homo sapiens branched chain aminotransferase 1, cytosolic (BCAT1), mRNA [NM_005504] |
| | b3 | Homo sapiens chemokine-like factor (CKLF), transcript variant 4, mRNA [NM_181641] |
| | b4 | Homo sapiens chemokine-like factor (CKLF), transcript variant 5, mRNA [NM_001040138] |
| | b5 | Homo sapiens chemokine-like factor (CKLF), transcript variant 1, mRNA [NM_016951] |
| | b6 | Homo sapiens CDC28 protein kinase regulatory subunit 2 (CKS2), mRNA [NM_001827] |
| | b7 | Homo sapiens GLI family zinc finger 2 (GLI2), mRNA [NM_005270] |
| | b8 | Homo sapiens microtubule-associated protein, RP/EB family, member 2 (MAPRE2), |
| | | transcript variant 1, mRNA [NM_014268] |
| | b9 | microtubule-associated protein, RP/EB family, member 2 [Source; HGNC Symbol; Ace: 6891] |
| | | [ENST00000300249] |
| | b10 | Homo sapiens v-myc myelocytomatosis viral oncogene homolog (avian) (MYC), mRNA |
| | | [NM_002467] |
| | b11 | Homo sapiens polo-like kinase 1 (Drosophila) (PLK1), mRNA [NM_005030] |
| | b12 | Homo sapiens polo-like kinase 1 (Drosophila) (PLK1), mRNA [NM_005030] |
| | b13 | Homo sapiens S-phase kinase-associated protein 2 (p45) (SKP2), transcript variant 2, |
| | | mRNA [NM_032637] |
| | b14 | Home sapiens tumor-associated calcium signal transducer 2 (TACSTD2), mRNA |
| | | [NM_002353] |
| | b15 | Homo sapiens tumor necrosis factor receptor superfamily, member 17 (TNFRSF17), mRNA |
| | | [NM_001192] |
| | b16 | Homo sapiens WD repeat domain 12 (WDR12), mRNA [NM_018256] |

R*: Ratio to control (times)

TABLE 3

| ID | GeneSymbol | Description | GenbankAccession | PubMed ID | log2 [ratio] | R* | p-value |
|---|---|---|---|---|---|---|---|
| A_23_P105138 | CAT | c1 | NM_001752 | 8099875 | 0.589 | 1.505 | 0.001 |
| A_23_P128215 | SOCS2 | c2 | NM_003877 | 9266833 | 0.783 | 1.720 | 0.016 |
| A_23_P253932 | FAIM | c3 | NM_001033030 | 10075978 | 0.740 | 1.670 | 0.000 |
| A_23_P56922 | HSPE1 | c4 | NM_002157 | 11282911 | 0.615 | 1.531 | 0.019 |
| A_23_P64129 | HTATIP2 | c5 | NM_006410 | 11282911 | 0.795 | 1.735 | 0.010 |
| A_23_P72285 | YWHAE | c6 | NM_006761 | 17409426 | 0.613 | 1.529 | 0.019 |
| A_23_P91504 | ITSN1 | c7 | AF064244 | 17405881 | 1.802 | 3.487 | 0.048 |
| A_24_P937256 | RELA | c8 | NM_001145138 | 9342183 | 0.688 | 1.611 | 0.039 |

| | | |
|---|---|---|
| Description | c1 | Homo sapiens catalase (CAT), mRNA [NM_001752] |
| | c2 | Homo sapiens suppressor of cytokine signaling 2 (SOCS2), mRNA [NM_003877] |
| | c3 | Homo sapiens Fas apoptotio inhibitory inhibitory molecule (FAIM), transcript variant 1, mRNA |
| | | [NM_001033030] |
| | c4 | Homo sapiens heat shock 10 kDa protein 1 (chaperonin 10) (HSPE1), mRNA |
| | | [NM_002157] |
| | c5 | Homo sapiens HIV-1 Tat interactive protein 2, 30 kDa (HTATIP2), transcript variant 2, |
| | | mRNA [NM_006410] |
| | c6 | Homo sapiens tyrosine 3-monooxygenase/tryptophan 5-monooxygenase activation |
| | | protein, epsilon polypeptide (YWHAE), transcript variant 1, mRNA [NM_006761] |
| | c7 | intersectin 1 (SH3 domain protein) [Source: HGNC Symbol; Acc; 6183] |
| | | [ENST00000381318] |
| | c8 | Homo sapiens v-rel reticuloendotheliosis viral oncogene homolog A (avian) (RELA), |
| | | transcript variant 2, mRNA [NM_001145138] |

R*: Ratio to control (times)

(Second Gene Group)

TABLE 4

| ID | GeneSymbol | Description | GenbankAccession | PubMed ID | log2 [ratio] | R* | p-value |
|---|---|---|---|---|---|---|---|
| A_23_P207003 | SEPT4 | d1 | NM_004574 | 11146656 | −0.990 | 0.503 | 0.006 |
| A_23_P206077 | AEN | d2 | NM_022767 | 18264133 | −1.253 | 0.420 | 0.003 |
| A_32_P75094 | AIFM2 | d3 | NM_032797 | 11980907 | −1.327 | 0.399 | 0.000 |
| A_23_P258194 | AIFM3 | d4 | NM_001018060 | 15764604 | −0.761 | 0.590 | 0.008 |
| A_23_P36611 | APAF1 | d5 | NM_181861 | 9267021 | −1.253 | 0.420 | 0.008 |
| A_23_P85024 | ARHGEF6 | d6 | NM_004840 | 11594864 | −1.889 | 0.270 | 0.025 |
| A_23_P145357 | BAK1 | d7 | NM_001188 | 7715730 | −1.313 | 0.402 | 0.001 |
| A_23_P208706 | BAX | d8 | NM_138764 | 12529375 | −1.143 | 0.453 | 0.028 |
| A_23_P346311 | BAX | d9 | NM_138764 | 12529375 | −0.969 | 0.511 | 0.038 |
| A_23_P382775 | BBC3 | d10 | NM_014417 | 11572983 | −1.666 | 0.315 | 0.001 |
| A_24_P305312 | BBC3 | d11 | NM_014417 | 11572983 | −1.452 | 0.366 | 0.002 |
| A_23_P115286 | BCL10 | d12 | NM_003921 | 10187815 | −0.883 | 0.542 | 0.002 |
| A_24_P19983 | BCL10 | d13 | NM_003921 | 10187815 | −0.810 | 0.571 | 0.017 |
| A_24_P122921 | BCL2L11 | d14 | NM_138621 | 11997495 | −0.652 | 0.636 | 0.004 |
| A_23_P128050 | BCL2L14 | d15 | NM_030766 | 11054413 | −0.751 | 0.594 | 0.019 |
| A_23_P404667 | BIK | d16 | NM_001197 | 7478623 | −1.748 | 0.298 | 0.003 |
| A_23_P23960 | BLOC1S2 | d17 | NM_001001342 | 18188704 | −1.336 | 0.396 | 0.001 |
| A_24_P295543 | BLOC1S2 | d18 | NM_001001342 | 18188704 | −1.381 | 0.384 | 0.001 |
| A_23_P138635 | BNIP3 | d19 | NM_004052 | 9396766 | −1.418 | 0.374 | 0.000 |
| A_23_P134925 | BNIP3L | d20 | NM_004331 | 9396766 | −0.661 | 0.633 | 0.002 |
| A_23_P87560 | BTG1 | d21 | NM_001731 | 9690562 | −1.265 | 0.416 | 0.003 |
| A_23_P15247 | C16orf5 | d22 | NM_013399 | 17598062 | −0.947 | 0.519 | 0.009 |
| A_23_P202978 | CASP1 | d23 | NM_033292 | 8242741 | −0.642 | 0.641 | 0.004 |
| A_23_P209408 | CASP10 | d24 | NM_032977 | 8755496 | −0.857 | 0.552 | 0 012 |
| A_24_P139993 | CASP10 | d25 | NM_032977 | 8755496 | −0.716 | 0.609 | 0.017 |
| A_23_P59210 | CDKN1A | d26 | NM_000389 | 8118801 | −1.187 | 0.439 | 0.000 |
| A_24_P89457 | CDKN1A | d27 | NM_078467 | 8118801 | −1.768 | 0.294 | 0.004 |
| A_23_P143242 | CEBPB | d28 | NM_005194 | 20981511 | −0.697 | 0.617 | 0.003 |
| A_23_P411296 | CEBPB | d29 | NM_005194 | 20961511 | −1.385 | 0.383 | 0.002 |
| A_23_P92025 | CIDEC | d30 | NM_022094 | 12429024 | −0.754 | 0.593 | 0.012 |

| | | |
|---|---|---|
| Description | d1 | Homo sapiens septin 4 (SEPT4), transcript variant 1, mRNA [NM_004574] |
| | d2 | Homo sapiens apoptosis enhancing nuclease (AEN), mRNA [NM_022767] |
| | d3 | Homo sapiens apoptosis-inducing factor, mitochondrion-associated, 2 (AIFM2), nuclear gene encoding |
| | | mitochondrial protein, mRNA [NM_032797] |
| | d4 | Homo sapiens apoptosis-inducing factor, mitochondrion-associated, 3 (AIFM3), nuclear gene encoding |
| | | mitochondrial protein, transcript variant 2, mRNA [NM_001018060] |
| | d5 | Homo sapiens apoptotic peptidase activating factor 1 (APAF1), transcript variant 3, mRNA [NM_181861] |
| | d6 | Homo sapiens Rac/Cdc42 guanine nucleotide exchange factor (GEF) 6 (ARHGEF6), mRNA [NM_004840] |
| | d7 | Homo sapiens BCL2-antagonist/killer 1 (BAK1), mRNA [NM_001188] |
| | d8 | Homo sapiens BCL2-associated X protein (BAX), transcript variant sigma, mRNA [NM_138764] |
| | d9 | Homo sapiens BCL2-associated X protein (BAX), transcript variant sigma, mRNA [NM_138764] |
| | d10 | Homo sapiens BCL2 binding component 3 (BBC3), transcript variant 4, mRNA [NM_014417] |
| | d11 | Homo sapiens BCL2 binding component 3 (BBC3), transcript variant 4, mRNA [NM_014417] |
| | d12 | Homo sapiens B-cell CLL/lymphoma 10 (BCL10), mRNA [NM_003921] |
| | d13 | Homo sapiens B-cell CLL/lymphoma 10 (BCL10), mRNA [NM_003921] |
| | d14 | Homo sapiens BCL2-like 11 (apoptosis facilitator) (BCL2L11), transcript variant 1, mRNA [NM_138621] |
| | d15 | Homo sapiens BCL2-like 14 (apoptosis facilitator) (BCL2L14), transcript variant 2, mRNA [NM_030766] |
| | d16 | Homo sapiens BCL2-interacting killer (apoptosis-inducing) (BIK), mRNA [NM_001197] |
| | d17 | Homo sapiens biogenesis of lysosomal organelles complex-1, subunit 2 (BLOC1S2), transcript variant 2, |
| | | mRNA [NM_001001342] |
| | d18 | Homo sapiens biogenesis of lysosomal organelles complex-1, subunit 2 (BLOC1S2), transcript variant 2, |
| | | mRNA [NM_001001342] |
| | d19 | Homo sapiens BCL2/adenovirus E1B 19 kDa interacting protein 3 (BNIP3), nuclear gene encoding |
| | | mitochondrial protein, mRNA [NM_004052] |
| | d20 | Homo sapiens BCL2/adenovirus E1B 19 kDa interacting protein 3-like (BNIP3L), mRNA [NM_004331] |
| | d21 | Homo sapiens B-cell translocation gene 1, anti-proliferative (BTG1), mRNA [NM_001731] |
| | d22 | Homo sapiens chromosome 16 open reading frame 5 (C16orf5), mRNA [NM_013399] |
| | d23 | Homo sapiens caspase 1, apoptosis-related cysteine peptidase (interleukin 1, beta, convertase) (CASP1), |
| | | transcript variant alpha, mRNA [NM_033292] |
| | d24 | Homo sapiens caspase 10, apoptosis-related cysteine peptidase (CASP10), transcript variant D. mRNA |
| | | [NM_032977] |
| | d25 | Homo sapiens caspase 10, apoptosis-related cysteine peptidase (CASP10), transcript variant D, mRNA |
| | | [NM_032977] |
| | d28 | Homo sapiens cyclin-dependent kinase inhibitor 1A (p21, Cip1) (CDKN1A), transcript variant 1, mRNA |
| | | [NM_000389] |
| | d27 | Homo sapiens cyclin-dependent kinase inhibitor 1A (p21, Cip1) (CDKN1A), transcript variant 2, mRNA |
| | | [NM_078467] |
| | d28 | Homo sapiens CCAAT/enhancer binding protein (C/EBP), beta (CEBPB), mRNA [NM_005194] |
| | d29 | Homo sapiens CCAAT/onhancer binding protein (C/EBP), beta (CEBPB), mRNA [NM_005194] |
| | d30 | Homo sapiens cell death-inducing DFFA-like effector c (CIDEC), mRNA [NM_022094] |

R*: Ratio to control (times)

TABLE 5

| ID | GeneSymbol | Description | GenbankAccession | PubMed ID | log2 [ratio] | R* | p-value |
|---|---|---|---|---|---|---|---|
| A_23_P21134 | DDIT3 | e1 | NM_004083 | 8898082 | −2.249 | 0.210 | 0.001 |
| A_24_P207479 | DEDD2 | e2 | NM_133328 | 11741985 | −0.744 | 0.597 | 0.016 |
| A_24_P4661 | EI24 | e3 | NM_004879 | 10594026 | −0.618 | 0.652 | 0.002 |
| A_23_P63896 | FAS | e4 | NM_000043 | 1713127 | −1.457 | 0.364 | 0.005 |
| A_23_P110052 | FOXL2 | e5 | NM_023067 | 16153597 | −1.504 | 0.352 | 0.001 |
| A_23_P160025 | IFI16 | e6 | NM_005531 | 14990579 | −0.669 | 0.629 | 0.001 |
| A_23_P217866 | IFI16 | e7 | NM_005531 | 14990579 | −0.773 | 0.585 | 0.001 |
| A_23_P151294 | IFNG | e8 | NM_000619 | 15115612 | −0.710 | 0.611 | 0.031 |
| A_23_P303210 | IKBIP | e9 | NM_153687 | 15389287 | −1.095 | 0.468 | 0.003 |
| A_23_P53467 | IKBIP | e10 | NM_201612 | 15389287 | −0.675 | 0.626 | 0.023 |
| A_23_P72096 | ILIA | e11 | NM_000575 | 1924307 | −2.211 | 0.216 | 0.001 |
| A_32_P230808 | KNG1 | e12 | NM_000893 | 11099478 | −1.484 | 0.358 | 0.008 |
| A_24_P284523 | MAP3K10 | e13 | NM_002446 | 10801775 | −0.810 | 0.570 | 0.029 |
| A_23_P89550 | NLRP1 | e14 | NM_033004 | 15212762 | −1.048 | 0.483 | 0.005 |
| A_24_P83615 | NLRP1 | e15 | NM_001033053 | 15212762 | −0.841 | 0.558 | 0.010 |
| A_23_P128230 | NR4A1 | e16 | NM_002135 | 12030839 | −1.727 | 0.302 | 0.004 |
| A_23_P360797 | NTF3 | e17 | NM_002527 | 21720807 | −1.024 | 0.492 | 0.005 |
| A_23_P34804 | NTRK1 | e18 | NM_002529 | 9136990 | −1.936 | 0.261 | 0.012 |
| A_24_P273157 | OBSCN | e19 | NM_052843 | 22441987 | −0.613 | 0.654 | 0.019 |
| A_23_P25354 | P2RX7 | e20 | NM_002562 | 9843914 | −0.834 | 0.561 | 0.017 |
| A_24_P319113 | P2RX7 | e21 | NM_002562 | 9843914 | −0.772 | 0.586 | 0.024 |
| A_23_P214950 | PERP | e22 | NM_022121 | 10733530 | −0.941 | 0.521 | 0.002 |
| A_23_P62959 | PHLDA3 | e23 | NM_012396 | 19203586 | −1.884 | 0.271 | 0.001 |
| A_23_P207999 | PMAIP1 | e24 | NM_021127 | 10807576 | −1.540 | 0.344 | 0.003 |
| A_23_P358944 | PML | e25 | NM_033244 | 9583681 | −0.770 | 0.586 | 0.003 |
| A_24_P261169 | SEMA4D | e26 | NM_006378 | 22198439 | −0.842 | 0.558 | 0.009 |
| A_23_P169738 | SOX7 | e27 | NM_031439 | 19108950 | −1.464 | 0.362 | 0.001 |
| A_23_P306987 | SOX7 | e28 | NM_031439 | 19108950 | −1.566 | 0.338 | 0.002 |
| A_24_P226782 | SOX7 | e29 | NM_031439 | 19108950 | −0.720 | 0.607 | 0.020 |
| A_23_P252817 | SST | e30 | NM_001048 | 10433861 | −0.587 | 0.666 | 0.045 |

| | | |
|---|---|---|
| Description | e1 | Homo sapiens DNA-damage-inducible transcript 3 (DDIT3), mRNA [NM_004083] |
| | e2 | Homo sapiens death effector domain containing 2 (DEDD2), mRNA [NM_133328] |
| | e3 | Homo sapiens etoposide induced 2, 4 mRNA (EI24), transcript variant 1, mRNA [NM_004879] |
| | e4 | Homo sapiens Fas (TNF receptor superfamily, member 6) (FAS), transcript variant 1, mRNA [NM_000043] |
| | e5 | Homo sapiens forkhead box L2 (FOXL2), mRNA [NM_023067] |
| | e6 | Homo sapiens interferon, gamma-inducible protein 16 (IFI16), mRNA [NM_005531] |
| | e7 | Homo sapiens interferon, gamma-inducible protein 16 (IFI16), mRNA [NM_005531] |
| | e8 | Homo sapiens interferon, gamma (IFNG), mRNA [NM_000619] |
| | e9 | Homo sapiens IKBKB interacting protein (IKBIP), transcript variant 1, mRNA [NM_153687] |
| | e10 | Homo sapiens IKBKB interacting protein (IKBIP), transcript variant 2, mRNA [NM_201612] |
| | e11 | Homo sapiens interleukin 1, alpha (1L1A), mRNA [NM_000575] |
| | e12 | Homo sapiens kininogen 1 (KNG1), transcript variant 2, mRNA [NM_000893] |
| | e13 | Homo sapiens mitogen-activated protein kinase kinase kinase 10 (MAP3K10), mRNA [NM_002446] |
| | e14 | Homo sapiens NLR family, pyrin domain containing 1 (NLRP1), transcript variant 1, mRNA [NM_033004] |
| | e15 | Homo sapiens NLR family, pyrin domain containing 1 (NLRP1), transcript variant 5, mRNA |
| | e16 | Homo sapiens nuclear receptor subfamily 4, group A, member 1 (NR4A1), transcript variant 1, mRNA |
| | e17 | Homo sapiens neurotraphin 3 (NTF3), transcript variant 2, mRNA [NM_002527] |
| | e18 | Homo sapiens neurotrophic tyrosine kinase, receptor, type 1 (NTRK1), transcript variant 2, mRNA |
| | e19 | Homo sapiens obscurin, cytoskeletal calmodulin and titin-interacting RhoGEF (OBSCN), transcript variant |
| | | 1, mRNA [NM_052843] |
| | e20 | Homo sapiens purinergic receptor P2X, ligand-gated ion channel, 7 (P2RX7), mRNA [NM_002562] |
| | e21 | Homo sapiens purinergic receptor P2X, ligand-gated ion channel, 7 (P2RX7), mRNA [NM_002562] |
| | e22 | Homo sapiens PERP, TP53 apoptosis effector (PERP), mRNA [NM_022121] |
| | e23 | Homo sapiens pleckstrin homology-like domain, family A, member 3 (PHLDA3), mRNA [NM_012396] |
| | e24 | Homo sapiens phorbol-12-myristate-13-acetate-induced protein 1 (PMAIP1), mRNA [NM_021127] |
| | e25 | Homo sapiens promyelocytic leukemia (PML), transcript variant 5, mRNA [NM_033244] |
| | e26 | Homo sapiens sema domain, immunoglobulin domain (Ig), transmembrane domain (TM) and short |
| | | cytoplasmic domain (semaphorin) 4D (SEMA4D), transcript variant 1, mRNA [NM_006378] |
| | e27 | Homo sapiens SRY (sex determining region Y)-box 7 (SOX7), mRNA [NM_031439] |
| | e28 | Homo sapiens SRY (sex determining region Y)-box 7 (SOX7), mRNA [NM_031439] |
| | e29 | Homo sapiens SRY (sex determining region Y)-box 7 (SOX7), mRNA [NM_031439] |
| | e30 | Homo sapiens somatostatin (SST), mRNA [NM_001048] |

R*: Ratio to control (times)

TABLE 6

| ID | GeneSymbol | Description | GenbankAccession | PubMed ID | log2 [ratio] | R* | p-value |
|---|---|---|---|---|---|---|---|
| A_23_P82550 | STK17A | f1 | NM_004760 | 9786912 | −0.683 | 0.623 | 0.001 |
| A_23_P88404 | TGFB3 | f2 | NM_003239 | 8753879 | −0.761 | 0.590 | 0.001 |
| A_23_P92499 | TLR2 | f3 | NM_003264 | 10426996 | −0.662 | 0.632 | 0.002 |
| A_23_P255653 | TNFRSF10A | f4 | NM_003844 | 22246181 | −1.314 | 0.402 | 0.007 |
| A_23_P169030 | TNFRSF10B | f5 | NM_003842 | 22789540 | −0.653 | 0.636 | 0.004 |
| A_24_P218265 | TNFRSF10B | f6 | NM_003842 | 22789540 | −1.073 | 0.475 | 0.003 |
| A_23_P126844 | TNFRSF25 | f7 | NM_148965 | 8875942 | −0.909 | 0.532 | 0.003 |
| A_23_P94754 | TNFSF15 | f8 | NM_005118 | 11911831 | −1.327 | 0.399 | 0.046 |
| A_23_P5392 | TP5313 | f9 | NM_004881 | 8631304 | −1.241 | 0.423 | 0.000 |
| A_23_P168882 | TP53INP1 | f10 | NM_033285 | 11511362 | −1.339 | 0.395 | 0.009 |
| A_23_P740131 | TP73 | f11 | NM_005427 | 10391251 | −0.843 | 0.557 | 0.007 |
| A_24_P413470 | TP73 | f12 | NM_005427 | 10391251 | −1.054 | 0.482 | 0.002 |
| A_23_P502553 | TRIM35 | f13 | NM_171982 | 14662771 | −0.695 | 0.618 | 0.004 |
| A_24_P639629 | ZMAT3 | f14 | NM_152240 | 9250682 | −0.612 | 0.654 | 0.012 |

| | | |
|---|---|---|
| Description | f1 | Homo sapiens serine/threonine kinase 17a (STK17A), mRNA [NM_004760] |
| | f2 | Homo sapiens transforming growth factor, beta 3 (TGFB3), mRNA [NM_003239] |
| | f3 | Homo sapiens toll-like receptor 2 (TLR2), mRNA [NM_003264] |
| | f4 | Homo sapiens tumor necrosis factor receptor superfamily, member 10a (TNFRSF10A), mRNA |
| | f5 | Homo sapiens tumor necrosis factor receptor superfamily, member 10b (TNFRSF10B), transcript variant |
| | | 1, mRNA [NM_003842] |
| | f6 | Homo sapiens tumor necrosis factor receptor superfamily, member 10b (TNFRSF10B), transcript variant |
| | | 1, mRNA [NM_003842] |
| | f7 | Homo sapiens tumor necrosis factor receptor superfamily, member 25 (TNFRSF25), transcript variant 1, |
| | | mRNA [NM_148965] |
| | f8 | Homo sapiens tumor necrosis factor (ligand) superfarnily, member 15 (TNFSF15), mRNA [NM_005118] |
| | f9 | Homo sapiens tumor protein p53 inducible protein 3 (TP53I3), transcript variant 1, mRNA [NM_004881] |
| | f10 | Homo sapiens tumor protein p53 inducible nuclear protein 1 (TP53INP1), transcript variant 1, mRNA |
| | | [NM_033285] |
| | f11 | Homo sapiens tumor protein p73 (TP73), transcript variant 1, mRNA [NM_005427] |
| | f12 | Homo sapiens tumor protein p73 (TP73), transcript variant 1, mRNA [NM_005427] |
| | f13 | Homo sapiens tripartite motif-containing 35 (TRIM35), transcript variant 2, mRNA [NM_171982] |
| | f14 | Homo sapiens zinc finger, matrin type 3 (ZMAT3), transcript variant 2, mRNA [NM_152240] |

R*: Ratio to control (times)

(6) A method for evaluating reactivity of animal cells relative to ethanolamine phosphate, the method comprising steps of:

administering ethanolamine phosphate to animal cells;

measuring an expression level of one or two or more genes selected from the first gene group described in claim 15 and/or an expression level of one or two or more genes selected from the second gene group described in (5) in the animal cells to which the ethanolamine phosphate was administered; and

evaluating the reactivity of the animal cells relative to ethanolamine phosphate based on the expression levels of the one or two or more genes as obtained in the previous step.

(7) A method for screening compounds that regulate action of ethanolamine phosphate, the method comprising steps of:

administering ethanolamine phosphate and one or two or more test compounds to animal cells;

measuring an expression level of one or two or more genes selected from the first gene group described in (5) and/or an expression level of one or two or more genes selected from the second gene group described in (5) in the animal cells; and

evaluating suppressing action or enhancing action of the one or two or more test compounds on the action of ethanolamine phosphate based on the expression levels of the one or two or more genes as obtained in the measuring.

(8) A method for screening ethanolamine phosphate-like compounds, the method comprising steps of:

administering, to animal cells, one or two or more test compounds selected from the group consisting of expression products of one or two or more genes selected from the first gene group described in (5), compounds that promote expression of these genes, compounds that act suppressively on expression products of one or two or more genes selected from the second gene group described in claim 15 and compounds that suppress expression of these genes; and

measuring one or two or more cell functions of the animal cells.

(9) A solid-phase carrier for screening regulators of the action of ethanolamine phosphate, the carrier comprising

a first region on which one or two or more probes for detecting one or two or more genes selected from the first gene group described in (5) are fixed; and

a second region on which one or two or more probes for detecting one or two or more genes selected from the second gene group described in (5) are fixed.

### BRIEF DESCRIPTION OF DRAWINGS

FIG. 1 shows proliferative effects of EP on an established line of normal human airway epithelial cells at different FBS concentrations, with absolute values shown on the left side and values relative to the control shown on the right side;

FIG. 2 shows images of cells under serum-free conditions, with EP 0 μM on the left side and EP 1000 μM on the right side;

FIG. 3 shows proliferative effects of EP on primary human airway epithelial cells;

FIG. 4 shows proliferative effects of EP on primary human lung fibroblast cells;

FIG. 5 shows proliferative effects of EP on primary human hair dermal papilla cells;

FIG. 6 shows proliferative effects of EP and minoxidil on primary human hair dermal papilla cells;

FIG. 7 shows proliferative effects of EP in cancer cells (A549, THP-1, HL-60) and model cells (HEK293);

FIG. 8 shows suppression of the cell proliferative effects of EP by a MEK 1/2 inhibitor;

FIG. 9 is a map of a metabolic process from EP and its metabolic derivatives to cell constituents;

FIG. 10 shows cell proliferation/toxicity effects of EP and its metabolic derivatives;

FIG. 11 shows results confirming cell proliferation suppressing effects of GABA receptor agonists, and the left side shows results obtained by GABA, while the right side shows results obtained by homotaurine;

FIG. 12 shows results confirming the cell proliferation suppressing effects of a GABA receptor antagonist;

FIG. 13 shows results confirming inhibitory effects of FBS and EP against increased caspase 3/7 activity induced by stress in the absence of serum;

FIG. 14 shows results confirming the barrier function promoting effects of EP in an established line of normal airway epithelial cells;

FIG. 15 shows results confirming the barrier function improvement effects of EP in a human airway epithelial cell cancer cell line Calu-3 (left side) and human normal primary keratinocytes HEKa (right side);

FIG. 16 is a box plot showing apoptosis-associated genes, expression of which was diminished by 2 times or more with EP, in comparison with H₂O₂-treated samples;

FIG. 17 is a scatter plot comparing expression of all apoptosis-associated probes after treatment with EP and H₂O₂;

FIG. 18 is a box plot showing oxidation-reduction associated genes, expression of which was diminished by two or more times with EP, in comparison with H₂O₂-treated samples;

FIG. 19 is a scatter plot comparing expression obtained by EP and H₂O₂ treatment of oxidation-reduction gene-associated probes;

FIG. 20 shows the number of genes, expressions of which were significantly altered by EP (1.5-fold or more or 1/1.5-fold or less, p<0.05);

FIG. 21 shows cell toxicity caused by hydrogen peroxide (H₂O₂) with or without EP pre-treatment;

FIG. 22 shows ELISA assay results for LPS-induced TNF-α secreted from THP-1 cells after pre-treatment with EP at various concentrations; and

FIG. 23 shows ELISA assay results for LPS-induced IL-8 secreted from THP-1 cells after pre-treatment with EP at various concentrations.

### DETAILED DESCRIPTION OF INVENTION

The present specification relates to an animal cell function enhancer having EP as an active ingredient, to a method for enhancing animal cell function using EP, and to a method for screening agents or inhibitors for animal cells and the like. EP is well known as a component of cell membranes, but the inventors have found that the actions of EP on cells are remarkably different from those of its close relatives on the metabolic pathway, and that EP can be used to effectively enhance the functions of animal cells. Enhancing the functions of animal cells includes increasing cell proliferation, suppressing apoptosis, improving barrier function and the like.

The present specification also clarifies the expression levels of genes associated with the action of EP, which has a variety of cell activating actions, and provides a novel use of EP.

The present specification relates to the actions of EP on cells when administered to cells, and to the promotion and suppression of the expression of gene groups that cause this action. Based on findings by the inventors about new actions of EP and about the expression states of these genes, it is possible to provide methods for screening gene expression control by EP, EP-like compounds, EP action enhancers and components of combined drugs using EP, and a solid-phase carrier for use in expression analysis of genes associated with the action of EP is also provided.

EP provides a wide range of animal cell function enhancement ranging from cell proliferation to apoptosis control, barrier function improvement and the like. It also has a hair growth action. Moreover, it also has an inflammatory cytokine suppressing action, anti-inflammatory action and antioxidant action.

Disclosures of the present specification are explained in detail below.

(Animal Cell Function Enhancer)

EP can be used as an active ingredient of an animal cell function enhancer. The EP may also be in the form of a phosphate ion forming a salt with sodium, potassium or another univalent metal ion or calcium, magnesium or another bivalent metal ion or the like on the phosphate group.

The animal cells are not particularly limited, and examples include cells of humans and non-human animals. The type of cell is also not particularly limited. The animal cells may be primary cells or an established cell line. Their derivation is also not particularly limited, but examples include cells derived from the lungs, airway and epidermis. In terms of cell morphology, either epithelial cells or fibroblasts or other cells of connective tissue are acceptable. The animal cells may be ES cells, pluripotent cells artificially induced from somatic cells (iPS cells), or cells that have been differentiated from such cells. The animal cells may also constitute all or part of a tissue or organ.

Of the animal cells, airway epithelial cells, lung fibroblasts, skin epidermal cells and hair dermal papilla cells are particularly desirable for cell proliferation by EP. These cells may be primary cells or established cell lines. The animal cells may also be animal cells collected from an individual animal.

The actions of EP are useful in animal cells, and particularly in the cells of humans and other mammals. Based on these actions of EP, various actions are expressed in animal cells when EP itself is administered in various forms, either externally, vascularly through the blood vessels or the like, orally, or by infusion or injection.

(Cell Proliferation Function)

One function of animal cells that is enhanced by a cell function enhancer is the cell proliferation function. One feature of the cell proliferation function of EP is that EP can ensure not only cell proliferation in the presence of serum or various growth factors and other growth factors, but also animal cell proliferation without serum or growth factors. In other words, it is possible to ensure proliferation of animal cells by adding EP to basal medium containing only sugar and other carbon sources and inorganic salts. These actions are not obtained with ethanolamine, phosphorylcholine, CDP-choline, triethanolamine or diethanolamine, which are related compounds on the metabolic pathway of EP.

The serum here is not particularly limited as long as it is derived from animal blood, and typical examples include serum derived from the blood of various animals including fetal calves, newborn calves, calves, adult cattle and other cattle, goats, chickens, pigs and the like. The growth factors are also not particularly limited, and examples include known endogenous proteins that can be used for cell proliferation. Typical examples include epithelial growth factor (EGF), insulin-like growth factor (IGF), transforming growth factor (TGF), neural growth factor (NGF), brain derived neurotrophic factor (BDNF), vascular endothelial growth factor (VEGF), granulocyte colony stimulating factor (G-CSF), granulocyte macrophage colony stimulating factor (GM-CSF), platelet derived growth factor (PDGF), erythropoietin (EPO), thrombopoietin (TPO), basic fibroblast growth factor (bFGF), hepatocyte growth factor (HGF) and the like.

Another feature of the cell function enhancer is that it selectively promotes proliferation of normal cells more than proliferation of atypical cells. A fact that the cell function enhancer exerts a proliferative function selectively on normal cells rather than cancer cells and other atypical cells, is particularly meaningful for diseases (various cancers for example, as well as conditions requiring wound healing) in which no adverse effects based on promoting proliferation of atypical cells and proliferation of normal cells, are important in terms of treatment and prevention especially when the cell function enhancer is administered to a living body.

“Atypical cells” are cells that deviate from the cell morphology ordinarily observed in normal cells. Typically, it means that the cells deviate from normal cell morphology when test cells are observed under an optical microscope. “Normal cells” here differ according to the type of cells, but may be evaluated according to the size of the nucleus or the like. The way in which atypical cells deviate from normal cells also differs according to the type of cells, but cancer cells are a typical example.

When EP is used as a cell proliferation agent, it can be added in a range that produces a cell proliferation function, and for example can be included in medium or the like in a range of 100 nM to 10 mM or preferably 1 μM to 1 mM or more preferably 10 μM to 1 mM.

(Barrier Function-Promoting Function)

Another function of animal cells that is enhanced by the cell function enhancer is the barrier function-promoting function of epithelial system cells. Epithelial cells are cells comprising tissue that serves a barrier function in the living body, and epithelial system cells are cells that serve a barrier function when they differentiate into epithelial cells. By enhancing the barrier function of epithelial system cells, it is possible to produce epithelial cells and tissue capable of resisting the intrusion of harmful substances and allergens. Since EP has a cell proliferation function in epithelial system cells, it can be used effectively to construct such tissues and cell populations. The concentration of EP when used a barrier function promoter is not particularly limited as long as it is sufficient to promote the barrier function, but for example it can be included in medium or the like in a concentration similar to that of the EP used as a cell proliferation agent.

In the present specification, a barrier function means more specifically an intrusion prevention mechanism against the intrusion of non-specific large molecules and irritating small molecules from the outside world. To inhibit the intrusion of foreign matter, the epithelium of living bodies forms structures called ‘tight junction's between epithelial cells, inhibiting the movement of molecules between adjacent cells (in the intercellular gaps). More specifically, the barrier function in the present specification is the action of inhibiting the intrusion of such foreign matter, or in other words the action of strengthening tight junctions. This barrier function, or in other words the strength of the tight junctions, is evaluated by measuring the transepithelial electrical resistance (TER) above and below the cell sheet. An increase in the tight junction strength is evaluated from a drop in the rate of permeation of dextran and other large molecules through the cell sheet.

(Apoptosis Suppressing Function)

Another function of animal cells that is enhanced by the cell function enhancer is the apoptosis suppressing function. Cell proliferation and cell functions can be obtained by exploiting the apoptosis suppressing function. Because EP targets the proliferative functions of normal cells as explained above, the apoptosis suppressing function can be called a normal cell apoptosis suppressing function. The concentration of EP when used an apoptosis suppressor is not particularly limited as long as it is sufficient to promote the apoptosis suppressing function, but for example it can be included in medium or the like in a concentration similar to that of the EP used as a cell proliferation agent.

(Cultured Cell Structure Preparation Agent)

Because EP has a cell proliferation function in fibroblasts as well as epithelial cells, and also has a barrier function promoting function, it is useful as a preparation agent for preparing various cultured cell structures formed by proliferation of epithelial cells, fibroblasts and both these kinds of cells. Examples include cell sheets used as skin substitutes and various other kinds of cultured cell structures. The form of the cultured cell structure is not particularly limited. Since the form of the cultured cell structure is not particularly limited, it can be selected appropriately according to the use of the cell structure and the type of tissue or organ to which it is applied. Examples include, sheet, bar, tubular and spherical forms and the like. Methods of preparing cultured cell structures are themselves well known to those skilled in the art, and EP can be used as a preparation agent for a cultured cell structure by including an effective amount of EP in the medium used to culture the cultured cell structure. The concentration of EP when used as a cultured cell structure preparation agent is not particularly limited as long as it is sufficient to promote the cell proliferation function and barrier function, but for example it can be included in medium or the like in a concentration similar to that of the EP used as a cell proliferation agent.

(Hair Growth Agent)

EP can be used as a hair growth agent. EP has a proliferative effect on hair dermal papilla cells. Proliferation of hair dermal papilla cells signifies a hair growth effect. Thus, a hair growth agent for external application to various kinds of skin including the scalp is provided, the agent containing EP as an active ingredient. The form of the hair growth agent is not particularly limited, and the form of a known topical preparation for application to the scalp and skin can be adopted. Examples include lotion, cream, essence, shampoo, rinse and the like. EP can be included in a range that produces a hair growth effect, such as for example 0.000001 mass % to 1 mass %. A preferred range is 0.0001 mass % to 0.1 mass %. The dosage and dosage regimen of the topical preparation are not particularly limited, but for example it may be applied externally between 1 and 3 times a day.

(Reagent for Evaluation or Screening)

Because EP acts on animal cells to enhance their function, it can be used as a reagent to evaluate the action of a test compound on animal cells by bringing the test compound into contact with animal cells in a presence of EP. More effective evaluation and screening are possible because the action of the test compound can be evaluated in cells whose function has been enhanced. The evaluation differs according to the type of cells, the type of test compound and the type of action, and can be set appropriately by a person skilled in the art according to an object of evaluation. When EP is used as a reagent for evaluating or screening a test compound, moreover, it may be used at a concentration at which the cell proliferation function, barrier function or other cell function is enhanced by EP, without any particular limitations, but for example it can be contained in medium or the like at a concentration similar to that of the EP used as a cell proliferation agent.

(Method for Enhancing Animal Cell Function, etc.)

The method for enhancing the function of animal cells disclosed in the present specification is a method for enhancing the function of animal cells by bringing animal cells into contact with EP outside the animal body. As explained above, EP can enhance cell functions. Outside the animal body, animal cells can be effectively proliferated, the barrier function can be promoted and apoptosis can be suppressed by bringing EP into contact with animal cells derived either from that individual animal or another animal Thus, this method is useful for proliferation and the like of cells when cells are used for various applications (fermentation, treatment (including prosthesis and transplantation), drugs). It is particularly useful in transplantation medicine in which cells are collected from an individual animal, cell function is enhanced with EP outside the body, and the cells are proliferated and then transplanted again into an individual animal for example.

The present method can also be implemented as a cell proliferation method for animal cells, a method for producing animal cells with a superior barrier function, or a method for producing animal cells with a superior apoptosis suppressing function. The present method can also be implemented as a hair promoting method in humans or non-human animals. Furthermore, the present method can be implemented as a method for improving the barrier function of animal cells or a method for suppressing apoptosis in animal cells.

(Screening Method, etc.)

The screening method disclosed in the present specification is a method for screening agents that act on animal cells, and may comprise a step of bringing a test compound into contact with the animal cells in the presence of EP and measuring its action in the animal cells. EP strengthens the function of animal cells. The action of a test compound on animal cells can also be measured more precisely or more practically in vitro by bringing the test compound into contact with animal cells in the presence of EP.

The test compound is not particularly limited, and examples include polypeptides, DNA, RNA and other nucleic acids, lipids, various other organic compounds, and various inorganic compounds including metal salt and other inorganic salt and the like. EP can normally be included in media for animal cells. The test compound can also typically be supplied temporarily, continuously or intermittently to the animal cell medium. The concentration of the EP for the cells is not particularly limited and may be set appropriately according to an object of screening. For example, it may be included in medium or the like at a concentration similar to that of the EP used as a cell proliferation agent.

The action of the test compound on the animal cells is not particularly limited. The action of promoting and the action of depressing various functions of cells are both included. Methods of measuring such actions can be implemented appropriately by a person skilled in the art according to the object of screening.

(Gene Expression Method)

The gene expression method disclosed in the present specification may comprise a step of administering EP to animal cells to thereby promote the expression of one or two or more genes selected from a first gene group and suppress the expression of one or two or more genes selected from a second gene group. With this method, it is possible to construct an expression state of a specific gene in animal cells by administering EP to animal cells. The expression state of this gene contributes to enhancing the functions of the animal cells. Thus, this expression method is also a method for manufacturing animal cells exhibiting such a gene expression state.

The animal cells are as explained previously. The method for administering the EP to the animal cells is not particularly limited, as long as the animal cells are brought into contact with EP. Typically, the animal cells may be cultured in medium containing EP.

In expression analysis of the total human genome by administration of EP to animal cells, expression of 801 and 1939 genes (probes) was changed by 1.5 times or more or 1/1.5 times or less (p<0.05), respectively. The results of functional analysis (GO analysis) of these genes are shown in Table 7 and Table 8. Table 7 shows the top 20 extraction results for gene functions with increased expression, while Table 8 shows the top 20 extraction results for gene functions with reduced expression. Expression analysis was performed using a dedicated analysis software ‘GeneSpring (Agilent)’.

(Functional Classification of Genes, Expression of Which was Significantly Increased by EP, Top 20 Ranked Based on p Values)

TABLE 7

| | | | | corrected | Count in | % Count in | Count in | % Count in |
|---|---|---|---|---|---|---|---|---|
| GO ID | GO ACCESSION | GO Term | p-value | p-value | Selection | Selection | Total | Total |
| 492 | GO:0000778 | condensed nuclear chromosome kinetochore | 2.62E−04 | 1.00E+00 | 3 | 4.48E−01 | 4 | 0.02 |
| 23582 | GO:0048514 | blood vessel morphogenesis | 5.05E−04 | 1.00E+00 | 18 | 2.69E+00 | 183 | 1.12 |
| 842 | GO:0001568 | blood vessel development | 6.23E−04 | 1.00E+00 | 20 | 2.99E+00 | 218 | 1.33 |
| 29817 | GO:0071824 | protein-DNA complex subunit organization | 7.23E−04 | 1.00E+00 | 11 | 1.64E+00 | 86 | 0.52 |
| 1162 | GO:0001944 | vasculature development | 9.25E−04 | 1.00E+00 | 20 | 2.99E+00 | 225 | 1.37 |
| 494 | GO:0000780 | condensed nuclear chromosome, centromeric | 1.04E−03 | 1.008+00 | 4 | 5.98E−01 | 12 | 0.07 |
| | | region | | | | | | |
| 3841 | GO:0005578 | proteinaceous extracellular matrix | 1.46E−03 | 1.00E+00 | 24 | 3.59E+00 | 302 | 1.84 |
| 14025 | GO:0031577 | spindle checkpoint | 1.46E−03 | 1.00E+00 | 4 | 5.98E−01 | 13 | 0.08 |
| 805 | GO:0001525 | angiogenesis | 1.53E−03 | 1.00E+00 | 14 | 2.09E+00 | 138 | 0.84 |
| 27990 | GO:0065004 | protein-DNA complex assembly | 1.64E−03 | 1.00E+00 | 10 | 1.49E+00 | 81 | 0.49 |
| 180 | GO:0000236 | mitotic prometaphase | 1.66E−03 | 1.00E+00 | 2 | 2.99E−01 | 2 | 0.01 |
| 3868 | GO:0005607 | laminin-2 complex | 1.66E−03 | 1.00E+00 | 2 | 2.99E−01 | 2 | 0.01 |
| 25924 | GO:0051984 | positive regulation of chromosome segregation | 1.66E−03 | 1.00E+00 | 2 | 2.99E−01 | 2 | 0.01 |
| 25927 | GO:0051987 | positive regulation of attachment of spindle | 1.66E−03 | 1.00E+00 | 2 | 2.99E−01 | 2 | 0.01 |
| | | microtubules to kinetochore | | | | | | |
| 17143 | GO:0034728 | nucleosome organization | 1.97E−03 | 1.00E+00 | 10 | 1.49E+00 | 83 | 0.51 |
| 2047 | GO:0002861 | regulation of inflammatory response to antigenic | 2.09E−03 | 1.00E+00 | 3 | 4.48E−01 | 7 | 0.04 |
| | | stimulus | | | | | | |
| 500 | GO:0000786\| | nucleosome | 2.32E−03 | 1.00E+00 | 8 | 1.20E+00 | 58 | 0.35 |
| | GO:0005718 | | | | | | | |
| 9243 | GO:0016043 | cellular component organization | 2.86E−03 | 1.00E+00 | 131 | 1.96E+01 | 2561 | 15.61 |
| 4449 | GO:0006334 | nucleosome assembly | 3.06E−03 | 1.00E+00 | 9 | 1.35E+00 | 74 | 0.45 |
| 13033 | GO:0030517 | negative regulation of axon extension | 3.24E−03 | 1.00E+00 | 3 | 4.48E−01 | 8 | 0.05 |

(Functional Classification of Genes, Expression of Which was Significantly Reduced by EP, Top 20 Ranked Based on p Values)

TABLE 8

| | | | | corrected | Count in | % Count in | Count in | % Count in |
|---|---|---|---|---|---|---|---|---|
| GO ID | GO ACCESSION | GO Term | p-value | p-value | Selection | Selection | Total | Total |
| 18960 | GO:0042981 | regulation of apoptosis | 1.51E−09 | 6.20E−05 | 123 | 8.23E+00 | 789 | 4.81 |
| 19043 | GO:0043067\| | regulation of programmed cell death | 2.82E−09 | 5.20E−05 | 123 | 8.23E+00 | 797 | 4.86 |
| | GO:0043070 | | | | | | | |
| 8102 | GO:0010941 | regulation of cell death | 3.55E−09 | 6.20E−05 | 123 | 823E+00 | 800 | 4.88 |
| 4934 | GO:0006917 | induction of apoptosis | 5.18E−09 | 6.20E−05 | 61 | 4.08E+00 | 309 | 1.88 |
| 8163 | GO:0012502\| | induction of programmed cell death | 5.89E−09 | 6.20E−05 | 61 | 4.08E+00 | 310 | 1.89 |
| | GO:0012503 | | | | | | | |
| 19041 | GO:0043065 | positive regulation of apoptosis | 1.44E−08 | 1.27E−04 | 74 | 4.95E+00 | 416 | 2.54 |
| 19044 | GO:0043068\| | positive regulation of programmed cell death | 1.97E−08 | 1.44E−04 | 74 | 4.95E+00 | 419 | 2.55 |
| | GO:0043071 | | | | | | | |
| 8103 | GO:0010942 | positive regulation of cell death | 2.19E−08 | 1.44E−04 | 74 | 4.95E+00 | 420 | 2.56 |
| 23921 | GO:0048869 | cellular developmental process | 1.50E−07 | 8.10E−04 | 203 | 1.36E+01 | 1582 | 9.64 |
| 12747 | GO:0030154 | cell differentiation | 1.54E−07 | 8.10E−04 | 199 | 1.33E+01 | 1545 | 9.42 |
| 24772 | GO:0050794\| | regulation of cellular process | 2.90E−07 | 1.39E−03 | 716 | 4.79E+01 | 6850 | 41.75 |
| | GO:0051244 | | | | | | | |
| 23581 | GO:0048513 | organ development | 5.03E−07 | 2.05E−03 | 183 | 1.22E+01 | 1420 | 8.66 |
| 14941 | GO:0032502 | developmental process | 5.07E−07 | 2.05E−03 | 343 | 2.29E+01 | 2976 | 18.14 |
| 27993 | GO:0065007 | biological regulation | 6.52E−07 | 2.45E−03 | 790 | 5.28E+01 | 7686 | 46.85 |
| 5222 | GO:0007275 | multicellular organismal development | 1.01E−06 | 3.54E−03 | 306 | 2.05E+01 | 2627 | 16.01 |
| 4466 | GO:0006355 | regulation of transcription, DNA-dependent | 1.65E−06 | 5.36E−03 | 206 | 1.35E+01 | 1664 | 10.14 |
| 25218 | GO:0051252 | regulation of RNA metabolic process | 1.73E−06 | 5.36E−03 | 211 | 1.41E+01 | 1713 | 10.44 |
| 24768 | GO:0050789\| | regulation of biological process | 2.21E−06 | 6.47E−03 | 756 | 5.06E+01 | 7365 | 44.89 |
| | GO:0050791 | | | | | | | |
| 4932 | GO:0006915\| | apoptosis | 4.13E−06 | 1.14E−02 | 81 | 5.42E+00 | 538 | 3.28 |
| | GO:0008632 | | | | | | | |
| 24771 | GO:0050793 | regulation of developmental process | 5.14E−06 | 1.35E−02 | 89 | 5.95E+00 | 610 | 3.72 |

As shown in Table 7, among the genes, expression of which was increased by EP administration, those associated with cell proliferation included gene functions associated with blood vessel formation and differentiation (GO: 0001568, 0048514, 0001944).

Moreover, as shown in Table 8, the genes, expression of which was reduced by EP administration, included gene functions associated with cell death and apoptosis (GO: 0042981, 0043067, 0043070, 0010941, 0006917, 0012502, 0012503, 0043065, 0043068, 0043071, 0010942, 0006915).

Of the functional molecules obtained by GO analysis shown in Table 7, the genes (29 probes, 20 genes) as functional molecules (GO: 0001944) are shown in Table 9. Table 10 shows those genes (16 probes, 12 genes) out of the genes associated with cell proliferation (GO: 0008283) that were not ranked in the top 20 in GO analysis but exhibited increased expression of 1.5-fold or more (p<0.05).

(Proliferation-Associated Genes, Expression of Which was Significantly Increased by EP (Angiogenesis-Associated Genes GO: 0001944))

TABLE 9

| ID | GeneSymbol | Description | GenbankAccession | log2 [ratio] | R* | p-value |
|---|---|---|---|---|---|---|
| A_23_P250554 | AGGF1 | a1 | NM_018046 | 0.607 | 1.523 | 0.012 |
| A_23_P146644 | ANXA2 | a2 | NM_001002857 | 0.742 | 1.673 | 0.007 |
| A_32_P94798 | ANXA2 | a3 | NM_001002857 | 0.691 | 1.614 | 0.018 |
| A_23_P110151 | ARHGAP24 | a4 | NM_001025616 | 0.740 | 1.670 | 0.035 |
| A_24_P388659 | ARHGAP24 | a5 | NM_001025616 | 0.803 | 1.745 | 0.011 |
| A_23_P54144 | BMP4 | a6 | NM_001202 | 0.864 | 1.820 | 0.003 |
| A_23_P55544 | CCBE1 | a7 | NM_133459 | 0.811 | 1.755 | 0.005 |
| A_32_P171043 | CCBE1 | a8 | NM_133459 | 0.918 | 1.890 | 0.006 |
| A_23_P211762 | COL8A1 | a9 | CN371015 | 0.716 | 1.643 | 0.035 |
| A_23_P69030 | COL8A1 | a10 | NM_001850 | 0.999 | 1.999 | 0.005 |
| A_24_P139152 | COL8A1 | a11 | AL359062 | 0.768 | 1.703 | 0.017 |
| A_23_P105803 | FGF9 | a12 | NM_002010 | 0.684 | 1.606 | 0.008 |
| A_23_P373521 | HAND2 | a13 | NM_021973 | 0.979 | 1.971 | 0.028 |
| A_24_P231104 | LEPR | a14 | NM_001003679 | 0.730 | 1.659 | 0.003 |
| A_23_P122216 | LOX | a15 | NM_002317 | 0.711 | 1.637 | 0.001 |
| A_23_P163787 | MMP2 | a16 | NM_004530 | 0.658 | 1.578 | 0.002 |
| A_23_P88589 | NR2F2 | a17 | NM_021005 | 0.733 | 1.662 | 0.005 |
| A_24_P313354 | NR2F2 | a18 | NM_021005 | 0.804 | 1.746 | 0.001 |
| A_24_P135322 | NRP1 | a19 | NM_001024629 | 0.735 | 1.664 | 0.003 |
| A_24_P928052 | NRP1 | a20 | NM_003873 | 0.993 | 1.990 | 0.001 |
| A_23_P201808 | PPAP2B | a21 | NM_003713 | 0.941 | 1.919 | 0.006 |
| A_23_P104881 | ROBO4 | a22 | NM_019055 | 0.836 | 1.785 | 0.011 |
| A_23_P344421 | ROBO4 | a23 | NM_019055 | 0.707 | 1.633 | 0.005 |
| A_23_P56933 | RTN4 | a24 | NM_020532 | 0.594 | 1.510 | 0.014 |
| A_23_P211957 | TGFBR2 | a25 | NM_001024847 | 0.708 | 1.634 | 0.009 |
| A_23_P337242 | TGFBR2 | a26 | NM_001024847 | 0.705 | 1.630 | 0.005 |
| A_23_P167096 | VEGFC | a27 | NM_005429 | 0.711 | 1.637 | 0.005 |
| A_23_P74380 | WARS2 | a28 | NM_201263 | 0.673 | 1.594 | 0.036 |
| A_23_P168909 | ZFPM2 | a29 | NM_012082 | 0.746 | 1.678 | 0.018 |

| | | |
|---|---|---|
| Description | a1 | Homo sapiens angiogenic factor with G patch and FHA domains 1 (AGGF1), |
| | | mRNA [NM_018046] |
| | a2 | Homo sapiens annexin A2 (ANXA2), transcript variant 2, mRNA [NM_001002857] |
| | a3 | Homo sapiens annexin A2 (ANXA2), transcript variant 2, mRNA [NM_001002857] |
| | a4 | Homo sapiens Rho GTPase activating protein 24 (ARHGAP24), transcript variant |
| | | 1, mRNA [NM_001025616] |
| | a5 | Homo sapiens Rho GTPase activating protein 24 (ARHGAP24), transcript variant |
| | | 1 mRNA [NM_001025616] |
| | a6 | Homo sapiens bone morphogenetic protein 4 (BMP4), transcript variant 1, mRNA |
| | | [NM_001202] |
| | a7 | Homo sapiens collagen and calcium binding EGF domains 1 (CCBE1), mRNA |
| | | [NM_133459] |
| | a8 | Homo sapiens collagen and calcium binding EGF domains 1 (CCBEI), mRNA |
| | | [NM_133459] |
| | a9 | collagen, type VIII, alpha 1 [Source: HGNC Symbol; Acc: 2215] |
| | | [ENST00000261037] |
| | a10 | Homo sapiens collagen, type VIII, alpha 1 (COL8A1), transcript variant 1, mRNA |
| | | [NM_001850] |
| | a11 | collagen, type VIII, alpha 1 [Source: HGNC Symbol: Ace: 2215] |
| | | [ENST00000261037] |
| | a12 | Homo sapiens fibroblast growth factor 9 (glia-activating factor) (FGF9), mRNA |
| | | [NM_002010] |
| | a13 | Homo sapiens heart and neural crest derivatives expressed 2 (HAND2), mRNA |
| | | [NM_021973] |
| | a14 | Homo sapiens leptin receptor (LEPR), transcript variant 3, mRNA |
| | | [NM_001003679] |
| | a15 | Homo sapiens lysyl oxidase (LOX), mRNA [NM_002317] |
| | a16 | Homo sapiens matrix metallopeptidase 2 (gelatinase A, 72 kDa gelatinase, 72 kDa |
| | | type IV collagenase) (MMP2), transcript variant 1, mRNA [NM_004530] |
| | a17 | Homo sapiens nuclear receptor subfamily 2, group F, member 2 (NR2F2), |
| | | transcript variant 1, mRNA [NM_021005] |
| | a18 | Homo sapiens nuclear receptor subfamily 2, group F, member 2 (NR2F2), |
| | | transcript variant 1, mRNA [NM_021005] |
| | a19 | Homo sapiens neuropilin 1 (NRP1), transcript variant 3, mRNA [NM_001024629] |
| | a20 | Homo sapiens neuropilin 1 (NRP1), transcript variant 1, mRNA [NM_003873] |
| | a21 | Homo sapiens phosphatidic acid phosphatase type 2B (PPAP2B), mRNA |
| | | [NM_003713] |
| | a22 | Homo sapiens roundabout homolog 4, magic roundabout (Drosophila) (ROBO4), |
| | | mRNA [NM_019055] |
| | a23 | Homo sapiens roundabout homolog 4, magic roundabout (Drosophila) (ROBO4), |
| | | mRNA [NM_019055] |
| | a24 | Homo sapiens reticulon 4 (RTN4), transcript variant 1, mRNA [NM_020532] |
| | a25 | Homo sapiens transforming growth factor, beta receptor II (70/80 kDa) |
| | | (TGFBR2), transcript variant 1, mRNA [NM_001024847] |
| | a26 | Homo sapiens transforming growth factor, beta receptor II (70/80 kDa) |
| | | (TGFBR2), transcript variant 1, mRNA [NM_001024847] |
| | a27 | Homo sapiens vascular endothelial growth factor C (VEGFG), mRNA [NM_005429] |
| | a28 | Homo sapiens tryptophanyl tRNA synthetase 2, mitochondrial (WARS2), nuclear |
| | | gene encoding mitochondrial protein, transcript variant 2, mRNA [NM_201263] |
| | a29 | Homo sapiens zinc finger protein, multitype 2 (ZFPM2), mRNA [NM_012082] |

R*: Ratio to control (times)

(Proliferation-Associated Genes, Expression of Which was Significantly Increased by EP (Cell Proliferation-Associated Genes GO: 0008283))

TABLE 10

| ID | GeneSymbol | Description | GenbankAccession | log2 [ratio] | R* | p-value |
|---|---|---|---|---|---|---|
| A_24_P302833 | APPL1 | b1 | NM_012096 | 0.785 | 1.723 | 0.005 |
| A_24_P935986 | BCAT1 | b2 | NM_005504 | 0.783 | 1.721 | 0.001 |
| A_23_P118061 | CKLF | b3 | NM_181641 | 0.586 | 1.501 | 0.040 |
| A_23_P206396 | CKLF | b4 | NM_001040138 | 0.679 | 1.601 | 0.006 |
| A_24_P215804 | CKLF | b5 | NM_016951 | 0.633 | 1.551 | 0.044 |
| A_23_P71727 | CKS2 | b6 | NM_001827 | 0.595 | 1.510 | 0.003 |
| A_23_P500034 | GLI2 | b7 | NM_005270 | 1.043 | 2.060 | 0.007 |
| A_24_P193911 | MAPRE2 | b8 | NM_014268 | 0.736 | 1.666 | 0.002 |
| A_23_P89835 | MAPRE2 | b9 | CR597075 | 0.621 | 1.538 | 0.023 |
| A_24_P38363 | MYC | b10 | NM_002467 | 0.754 | 1.686 | 0.025 |
| A_24_P313504 | PLK1 | b11 | NM_005030 | 0.955 | 1.939 | 0.004 |
| A_23_P118174 | PLK1 | b12 | NM_005030 | 1.291 | 2.447 | 0.000 |
| A_23_P156310 | SKP2 | b13 | NM_032637 | 0.615 | 1.531 | 0.001 |
| A_23_P149529 | TACSTD2 | b14 | NM_002353 | 0.591 | 1.507 | 0.003 |
| A_23_P37736 | TNFRSF17 | b15 | NM_001192 | 1.336 | 2.525 | 0.005 |
| A_23_P28625 | WDR12 | b16 | NM_018256 | 0.745 | 1.676 | 0.004 |

| | | |
|---|---|---|
| Description | b1 | Homo sapiens adaptor protein, phosphotyrosine interaction, PH domain and leucine zipper |
| | | containing 1 (APPL1), mRNA [NM_012096] |
| | b2 | Homo sapiens branched chain aminotransferase 1 cytosolic (BCAT1) mRNA [NM_005504] |
| | b3 | Homo sapiens chemokine-like factor (CKLF), transcript variant 4, mRNA [NM_181641] |
| | b4 | Homo sapiens chemokine-like factor (CKLF), transcript variant 5, mRNA [NM_001040138] |
| | b5 | Homo sapiens chemokine-like factor (CKLF), transcript variant 1, mRNA [NM_016951] |
| | b6 | Homo sapiens CDC28 protein kinase regulatory subunit 2 (CKS2), mRNA [NM_001827] |
| | b7 | Homo sapiens GLI family zinc finger 2 (GLI2), mRNA [NM_005270] |
| | b8 | Homo sapiens microtubule-associated protein, RP/EB family, member 2 (MAPRE2), |
| | | transcript variant 1, mRNA [NM_014268] |
| | b9 | microtubule-associated protein, RP/EB family, member 2 [Source: HGNC Symbol: Ace: 6891] |
| | | [ENST00000300249] |
| | b10 | Homo sapiens v-myc myelocytomatosis viral oncogene homolog (avian) (MYC), mRNA |
| | | [NM_002467] |
| | b11 | Homo sapiens polo-like kinase 1 (Drosophila) (PLK1), mRNA [NM_005030] |
| | b12 | Homo sapiens polo-like kinase 1 (Drosophila) (PLK1), mRNA [NM_005030] |
| | b13 | Homo sapiens S-phase kinase-associated protein 2 (p45) (SKP2), transcript variant 2, |
| | | mRNA [NM_032637] |
| | b14 | Homo sapiens tumor-associated calcium signal transducer 2 (TACSTD2), mRNA |
| | | [NM_002353] |
| | b15 | Homo sapiens tumor necrosis factor receptor superfamily, member 17 (TNFRSF17), mRNA |
| | | [NM_001192] |
| | b16 | Homo sapiens WD repeat domain 12 (WDR12), mRNA [NM_018256] |

R*: Ratio to control (times)

(Genes Having Apoptosis-promoting Action out of Apoptosis-Associated Genes, Expression of Which was Significantly Reduced by EP (Apoptosis Control-Associated Genes GO: 0042981))

TABLE 11

| ID | GeneSymbol | Description | GenbankAccession | PubMed ID | log2 [ratio] | R* | p-value |
|---|---|---|---|---|---|---|---|
| A_23_P207003 | SEPT4 | d1 | NM_004574 | 11146656 | −0.990 | 0.503 | 0.006 |
| A_23_P206077 | AEN | d2 | NM_022767 | 18264133 | −1.253 | 0.420 | 0.003 |
| A_32_P75094 | AIFM2 | d3 | NM_032797 | 11980907 | −1.327 | 0.399 | 0.000 |
| A_23_P258194 | AIFM3 | d4 | NM_001018060 | 15764604 | −0.761 | 0.590 | 0.008 |
| A_23_P36611 | APAF1 | d5 | NM_181861 | 9267021 | −1.253 | 0.420 | 0.008 |
| A_23_P85024 | ARHGEF6 | d6 | NM_004840 | 11564864 | −1.889 | 0.270 | 0.025 |
| A_23_P145357 | BAK1 | d7 | NM_001188 | 7715730 | −1.313 | 0.402 | 0.001 |
| A_23_P208706 | BAX | d8 | NM_138764 | 12529375 | −1.143 | 0.453 | 0.028 |
| A_23_P346311 | BAX | d9 | NM_138764 | 12529375 | −0.969 | 0.511 | 0.038 |
| A_23_P382775 | BBC3 | d10 | NM_014417 | 11572983 | −1.666 | 0.315 | 0 001 |
| A_24_P305312 | BBC3 | d11 | NM_014417 | 11572983 | −1.452 | 0.366 | 0.002 |
| A_23_P115286 | BCL10 | d12 | NM_003921 | 10187815 | −0.883 | 0.542 | 0.002 |
| A_24_P19983 | BCL10 | d13 | NM_003921 | 10187815 | −0.810 | 0.571 | 0.017 |
| A_24_P122921 | BCL2L11 | d14 | NM_138621 | 11997495 | −0.652 | 0.636 | 0.004 |
| A_23_P128050 | BCL2L14 | d15 | NM_030766 | 11054413 | −0.751 | 0.594 | 0.019 |
| A_23_P404667 | BIK | d16 | NM_001197 | 7478623 | −1.748 | 0.298 | 0.003 |
| A_23_P23960 | BLOC1S2 | d17 | NM_001001342 | 18188704 | −1.336 | 0.396 | 0.001 |
| A_24_P295543 | BLOC1S2 | d18 | NM_001001342 | 18188704 | −1.381 | 0.384 | 0.001 |
| A_23_P138635 | BNIP3 | d19 | NM_004052 | 9396766 | −1.418 | 0.374 | 0.000 |
| A_23_P134925 | BNIP3L | d20 | NM_004331 | 9396766 | −0.661 | 0.633 | 0.002 |
| A_23_P87560 | BTG1 | d21 | NM_001731 | 9690562 | −1.265 | 0.416 | 0.003 |
| A_23_P15247 | C16orf5 | d22 | NM_013399 | 17599062 | −0.947 | 0.519 | 0.009 |
| A_23_P202978 | CASP1 | d23 | NM_033292 | 8242741 | −0.642 | 0.641 | 0.004 |
| A_23_P209408 | CASP10 | d24 | NM_032977 | 8755496 | −0.857 | 0.552 | 0.012 |
| A_24_P139993 | CASP10 | d25 | NM_032977 | 8755496 | −0.716 | 0.609 | 0.017 |
| A_23_P59210 | CDKN1A | d26 | NM_000389 | 8118801 | −1.187 | 0.439 | 0.000 |
| A_24_P89457 | CDKN1A | d27 | NM_078467 | 8118601 | −1.768 | 0.294 | 0.004 |
| A_23_P143242 | CEBPB | d28 | NM_005194 | 20981511 | −0.697 | 0.617 | 0.003 |
| A_23_P411296 | CEBPB | d29 | NM_005194 | 20981511 | −1.385 | 0.383 | 0.002 |
| A_23_P92025 | GIDEC | d30 | NM_022094 | 12429024 | −0.754 | 0.593 | 0.012 |

| | | |
|---|---|---|
| Description | d1 | Homo sapiens septin 4 (SEPT4), transcript variant 1, mRNA [NM_004574] |
| | d2 | Homo sapiens apoptosis enhancing nuclease (AEN), mRNA [NM_022767] |
| | d3 | Homo sapiens apoptosis-inducing factor, mitochondrion-associated, 2 (AIFM2), nuclear gene encoding |
| | | mitochondrial protein, mRNA [NM_032797] |
| | d4 | Homo sapiens apoptosis-inducing factor, mitochondrion-associated, 3 (AIFM3), nuclear gene encoding |
| | | mitochondrial protein, transcript variant 2, mRNA [NM_001018060] |
| | d5 | Homo sapiens apoptotic peptidase activating factor 1 (APAF1), transcript variant 3, mRNA [NM_181861] |
| | d6 | Homo sapiens Rac/Cdc42 guanine nucleotide exchange factor (CEF) 6 (ARHGEF6), mRNA [NM_004840] |
| | d7 | Homo sapiens BCL2-antagonist/killer 1 (BAK1), mRNA [NM_001188] |
| | d8 | Homo sapiens BCL2-associated X protein (BAX), transcript variant sigma, mRNA [NM_138764] |
| | d9 | Homo sapiens BCL2-associated X protein (BAX), transcript variant sigma, mRNA [NM_138764] |
| | d10 | Homo sapiens BCL2 binding component 3 (BBC3), transcript variant 4, mRNA [NM_014417] |
| | d11 | Homo sapiens BCL2 binding component 3 (BBC3), transcript variant 4, mRNA [NM_014417] |
| | d12 | Homo sapiens B-cell CLL/lymphoma 10 (BCL10), mRNA [NM_003921] |
| | d13 | Homo sapiens B-cell CLL/lymphoma 10 (BCL10), mRNA [NM_003921] |
| | d14 | Homo sapiens BCL2-like 11 (apoptosis facilitator) (BCL2L11), transcript variant 1, mRNA [NM_138621] |
| | d15 | Homo sapiens BCL2-like 14 (apoptosis facilitator) (BCL2L14), transcript variant 2, mRNA [NM_030766] |
| | d16 | Homo sapiens BCL2-interacting killer (apoptosis-inducing) (BIK), mRNA [NM_001197] |
| | d17 | Homo sapiens biogenesis of lysosomal organelles complex-1, subunit 2 (BLOC1S2), transcript variant 2, |
| | | mRNA [NM_001001342] |
| | d18 | Homo sapiens biogenesis of lysosomal organelles complex-1, subunit 2 (BLOC1S2), transcript variant 2, |
| | | mRNA [NM_001001342] |
| | d19 | Homo sapiens BCL2/adenovirus E1B 19 kDa interacting protein 3 (BNIP3), nuclear gene encoding |
| | | mitochondrial protein, mRNA [NM_004052] |
| | d20 | Homo sapiens BCL2/adenovirus E1B 19 kDa interacting protein 3-like (BNIP3L), mRNA [NM_004331] |
| | d21 | Homo sapiens B-cell translocation gene 1, anti-proliferative (BTG1), mRNA [NM_001731] |
| | d22 | Homo sapiens chromosome 16 open reading frame 5 (C16orf5), mRNA [NM_013399] |
| | d23 | Homo sapiens caspase 1, apoptosis-related cysteine peptidase (interleukin 1, beta, convertase) (CASP1), |
| | | transcript variant alpha, mRNA [NM_033292] |
| | d24 | Homo sapiens caspase 10, apoptosis-related cysteine peptidase (CASP10), transcript variant D, mRNA |
| | | [NM_032977] |
| | d25 | Homo sapiens caspase 10, apoptosis-related cysteine peptidase (CASP10), transcript variant D, mRNA |
| | | [NM_032977] |
| | d26 | Homo sapiens cyclin-dependent kinase inhibitor 1A (p21, Cip1) (CDKN1A), transcript variant 1, mRNA |
| | | [NM_000389] |
| | d27 | Homo sapiens cyclin-dependent kinase inhibitor 1A (p21, Cip1) (CDKN1A), transcript variant 2, mRNA |
| | | [NM_078467] |
| | d28 | Homo sapiens CCAAT/enhancer binding protein (C/EBP), beta (CEBPB), mRNA [NM_005194] |
| | d29 | Homo sapiens CCAAT/enhancer binding protein (C/EBP), beta (CEBPB), mRNA [NM_005194] |
| | d30 | Homo sapiens cell death-inducing DFFA-like effector c (CIDEC), mRNA [NM_022094] |

R*: Ratio to control (times)

(Genes Having Apoptosis-Promoting Action out of Apoptosis-Associated Genes, Expression of Which was Significantly Reduced by EP (Apoptosis Control-Associated Genes GO: 0042981)

TABLE 12

| ID | GeneSymbol | Description | GonbankAccession | PubMed ID | log2 [ratio] | R* | p-value |
|---|---|---|---|---|---|---|---|
| A_23_P21134 | DDIT3 | e1 | NM_004083 | 8898082 | −2.249 | 0.210 | 0.001 |
| A_24_P207479 | DEDD2 | e2 | NM_133328 | 11741985 | −0.744 | 0.597 | 0.016 |
| A_24_P4661 | EI24 | e3 | NM_004879 | 10594026 | −0.618 | 0.652 | 0.002 |
| A_23_P63896 | FAS | e4 | NM_000043 | 1713127 | −1.457 | 0.364 | 0.005 |
| A_23_P110052 | FOXL2 | e5 | NM_023067 | 16153597 | −1.504 | 0.352 | 0.001 |
| A_23_P160025 | IFI16 | e6 | NM_005531 | 14990579 | −0.669 | 0.629 | 0.001 |
| A_23_P217869 | IFI16 | e7 | NM_005531 | 14990579 | −0.773 | 0.585 | 0.001 |
| A_23_P151294 | IFNG | e8 | NM_000619 | 15115612 | −0.710 | 0.611 | 0.031 |
| A_23_P303210 | IKBIP | e9 | NM_153687 | 15389287 | −1.095 | 0.468 | 0.003 |
| A_23_P53467 | IKBIP | e10 | NM_201612 | 15389287 | −0.675 | 0.626 | 0.023 |
| A_23_P72096 | IL1A | e11 | NM_000575 | 1924307 | −2.211 | 0.216 | 0.001 |
| A_32_P230808 | KNG1 | e12 | NM_000893 | 11099478 | −1.484 | 0.358 | 0.008 |
| A_24_P284523 | MAP3K10 | e13 | NM_002446 | 10801775 | −0.810 | 0.570 | 0.029 |
| A_23_P89550 | NLRP1 | e14 | NM_033004 | 15212762 | −1.048 | 0.483 | 0.005 |
| A_24_P83615 | NLRP1 | e15 | NM_001033053 | 15212762 | −0.841 | 0.558 | 0.010 |
| A_23_P128230 | NR4A1 | e16 | NM_002135 | 12030839 | −1.727 | 0.302 | 0.004 |
| A_23_P360797 | NTF3 | e17 | NM_002527 | 21720807 | −1.024 | 0.492 | 0.005 |
| A_23_P34804 | NTRK1 | e18 | NM_002529 | 9136990 | −1.936 | 0.261 | 0.012 |
| A_24_P273157 | OBSCN | e19 | NM_052843 | 22441987 | −0.613 | 0.654 | 0.019 |
| A_23_P25354 | P2RX7 | e20 | NM_002552 | 9843914 | −0.834 | 0.561 | 0.017 |
| A_24_P319113 | P2RX7 | e21 | NM_002562 | 9843914 | −0.772 | 0.586 | 0.024 |
| A_23_P214950 | PERP | e22 | NM_022121 | 10733530 | −0.941 | 0.521 | 0.002 |
| A_23_P62959 | PHLDA3 | e23 | NM_012396 | 19203586 | −1.884 | 0.271 | 0.001 |
| A_23_P207999 | PMAIP1 | e24 | NM_021127 | 10807576 | −1.540 | 0.344 | 0.003 |
| A_23_P358944 | PML | e25 | NM_033244 | 9583581 | −0.770 | 0.586 | 0.003 |
| A_24_P261169 | SEMA4D | e26 | NM_006378 | 22198439 | −0.842 | 0.558 | 0.009 |
| A_23_P169738 | SOX7 | e27 | NM_031439 | 19108950 | −1.464 | 0.362 | 0.001 |
| A_23_P306987 | SOX7 | e28 | NM_031439 | 19108950 | −1.566 | 0.338 | 0.002 |
| A_24_P226782 | SOX7 | e29 | NM_031439 | 19108950 | −0.720 | 0.607 | 0.020 |
| A_23_P252817 | SST | e30 | NM_001049 | 10433861 | −0.587 | 0.666 | 0.045 |

| | | |
|---|---|---|
| Description | e1 | Homo sapiens DNA-damage-inducible transcript 3 (DDIT3), mRNA [NM_004083] |
| | e2 | Homo sapiens death effector domain containing 2 (DEDD2), mRNA [NM_133328] |
| | e3 | Homo sapiens etoposide induced 2, 4 mRNA (EI24), transcript variant 1, mRNA [NM_004879] |
| | e4 | Homo sapiens Fas (TNF receptor superfamily, member 6) (FAS), transcript variant 1, mRNA [NM_000043] |
| | e5 | Homo sapiens forkhead box L2 (FOXL2), mRNA [NM_023067] |
| | e6 | Homo sapiens interferon, gamma-inducible protein 16 (IFI16), mRNA [NM_005531] |
| | e7 | Homo sapiens interferon, gamma-inducible protein 16 (IFI16), mRNA [NM_005531] |
| | e8 | Homo sapiens interferon, gamma (IFNG), mRNA [NM_000619] |
| | e9 | Homo sapiens IKBKB interacting protein (IKBIP), transcript variant 1, mRNA [NM_153687] |
| | e10 | Homo sapiens IKBKB interacting protein (IKBIP), transcript variant 2, mRNA [NM_201612] |
| | e11 | Homo sapiens interleukin 1, alpha (IL1A), mRNA [NM_000575] |
| | e12 | Homo sapiens kininogen 1 (KNG1), transcript variant 2, mRNA [NM_000893] |
| | e13 | Homo sapiens mitogen-activated protein kinase kinase kinase 10 (MAP3K10), mRNA [NM_002446] |
| | e14 | Homo sapiens NLR family, pyrin domain containing 1 (NLRP1), transcript variant 1, mRNA [NM_033004] |
| | e15 | Homo sapiens NLR family, pyrin domain containing 1 (NLRP1), transcript variant 5, mRNA |
| | e16 | Homo sapiens nuclear receptor subfamily 4, group A, member 1 (NR4A1), transcript variant 1 mRNA |
| | e17 | Homo sapiens neurotrophin 3 (NTF3), transcript variant 2, mRNA [NM_002527] |
| | e18 | Homo sapiens neurotrophic tyrosine kinase, receptor, type 1 (NTRK1), transcript variant 2, mRNA |
| | e19 | Homo sapiens obscurin cytoskeletal calmodulin and titin-interacting RhoGEF (OBSCN), transcript variant |
| | | 1, mRNA [NM_052843] |
| | e20 | Homo sapiens purinergic receptor P2X, ligand-gated ion channel, 7 (P2RX7), mRNA [NM_002562] |
| | e21 | Homo sapiens purinergic receptor P2X, ligand-gated ion channel, 7 (P2RX7), mRNA [NM_002562) |
| | e22 | Homo sapiens PERP, TP53 apoptosis effector (PERP) mRNA [NM_022121] |
| | e23 | Homo sapiens pleckstrin homology-like domain, family A, member 3 (PHLDA3), mRNA [NM_012396] |
| | e24 | Homo sapiens phorbol-12-myristate-13-acetate-induced protein 1 (PMAIP1), mRNA [NM_021127] |
| | e25 | Homo sapiens promyelocytic leukemia (PML), transcript variant 5, mRNA [NM_033244] |
| | e26 | Homo sapiens sema domain, immunoglobulin domain (Ig), transmembrane domain (TM) and short |
| | | cytoplasmic domain, (semaphorin) 4D (SEMA4D), transcript variant 1, mRNA [NM_006378] |
| | e27 | Homo sapiens SRY (sex determining region Y)-box 7 (SOX7), mRNA [NM_031439] |
| | e28 | Homo sapiens SRY (sex determining region Y)-box 7 (SOX7), mRNA [NM_031439] |
| | e29 | Homo sapiens SRY (sex determining region Y)-box 7 (SOX7), mRNA [NM_031439] |
| | e30 | Homo sapiens somatostatin (SST), mRNA [NM_001048] |

R*: Ratio to control (times)

(Genes Having Apoptosis-Promoting Action out of Apoptosis-Associated Genes, Expression of Which was Significantly Reduced by EP (Apoptosis Control-Associated Genes GO: 0042981)

TABLE 13

| ID | GeneSymbol | Description | GenbankAccession | PubMed ID | log2 [ratio] | R* | p-value |
|---|---|---|---|---|---|---|---|
| A_23_P82550 | STK17A | f1 | NM_004760 | 9786912 | −0.683 | 0.623 | 0.001 |
| A_23_P88404 | TGFB3 | f2 | NM_003239 | 8753879 | −0.761 | 0.590 | 0.001 |
| A_23_P92499 | TLR2 | f3 | NM_003264 | 10426996 | −0.662 | 0.632 | 0.002 |
| A_23_P255653 | TNFRSF10A | f4 | NM_003844 | 22246181 | −1.314 | 0.402 | 0.007 |
| A_23_P169030 | TNFRSF10B | f5 | NM_003842 | 22789540 | −0.653 | 0.636 | 0.004 |
| A_24_P218265 | TNFRSF10B | f6 | NM_003842 | 22789540 | −1.073 | 0.475 | 0.003 |
| A_23_P126844 | TNFRSF25 | f7 | NM_148965 | 8875942 | −0.909 | 0.532 | 0.003 |
| A_23_P94754 | TNFSF15 | f8 | NM_005118 | 11911831 | −1.327 | 0.399 | 0.046 |
| A_23_P5392 | TP53I3 | f9 | NM_004881 | 8631304 | −1.241 | 0.423 | 0.000 |
| A_23_P168882 | TP53INP1 | f10 | NM_033285 | 11511362 | −1.339 | 0.395 | 0.009 |
| A_23_P74081 | TP73 | f11 | NM_005427 | 10391251 | −0.843 | 0.557 | 0.007 |
| A_24_P413470 | TP73 | f12 | NM_005427 | 10391251 | −1.054 | 0.482 | 0.002 |
| A_23_P502553 | TRIM35 | f13 | NM_171982 | 14662771 | −0.695 | 0.618 | 0.004 |
| A_24_P639629 | ZMAT3 | f14 | NM_152240 | 9250682 | −0.612 | 0.554 | 0.012 |

| | | |
|---|---|---|
| Description | f1 | Homo sapiens serine/threonine kinase 17a (STK17A), mRNA [NM_004760] |
| | f2 | Homo sapiens transforming growth factor, beta 3 (TGFB3), mRNA [NM_003239] |
| | f3 | Homo sapiens toll-like receptor 2 (TLR2), mRNA [NM_003264] |
| | f4 | Homo sapiens tumor necrosis factor receptor superfamily, member 10a (TNFRSF10A), mRNA |
| | f5 | Homo sapiens tumor necrosis factor receptor superfamily, member 10b (TNFRSF10B), transcript variant |
| | | 1, mRNA [NM_003842] |
| | f6 | Homo sapiens tumor necrosis factor receptor superfamily, member 10b (TNFRSF10B), transcript variant |
| | | 1, mRNA [NM_003842] |
| | f7 | Homo sapiens tumor necrosis factor receptor superfamily, member 25 (TNFRSF25), transcript variant 1, |
| | | mRNA [NM_148965] |
| | f8 | Homo sapiens tumor necrosis factor (ligand) superfamily, member 15 (TNFSF15), mRNA [NM_005118] |
| | f9 | Homo sapiens tumor protein p53 inducible protein 3 (TP5313), transcript variant 1, mRNA [NM_004881] |
| | | Homo sapiens tumor protein p53 inducible nuclear protein 1 (TP53INP1), transcript variant 1, mRNA |
| | f10 | [NM_033285] |
| | f11 | Homo sapiens tumor protein p73 (TP73), transcript variant 1, mRNA [NM_005427] |
| | f12 | Homo sapiens tumor protein p73 (TP73), transcript variant 1, mRNA [NM_005427] |
| | f13 | Homo sapiens tripartite motif-containing 35 (TRIM35), transcript variant 2, mRNA [NM_171992] |
| | f14 | Homo sapiens zinc finger, matrin type 3 (ZMAT3), transcript variant 2, mRNA [NM_152240] |

R*: Ratio to control (times)

(Genes Having Apoptosis-Suppressing Action out of Apoptosis-Associated Genes, Expression of Which was Significantly Increased by EP (Apoptosis Control-Associated Genes GO: 0042981)

TABLE 14

| ID | GeneSymbol | Description | GenbankAccession | PubMed ID | log2 [ratio] | R* | p-value |
|---|---|---|---|---|---|---|---|
| A_23_P105138 | CAT | c1 | NM_001752 | 8099875 | 0.589 | 1.505 | 0.001 |
| A_23_P128215 | SOCS2 | c2 | NM_003877 | 9266833 | 0.783 | 1.720 | 0.016 |
| A_23_P253932 | FAIM | c3 | NM_001033030 | 10075978 | 0.740 | 1.670 | 0.000 |
| A_23_P56922 | HSPE1 | c4 | NM_002157 | 11282911 | 0.615 | 1.531 | 0.019 |
| A_23_P64129 | HTATIP2 | c5 | NM_006410 | 11282911 | 0.795 | 1.735 | 0.010 |
| A_23_P72285 | YWHAE | c6 | NM_006761 | 17409426 | 0.613 | 1.529 | 0.019 |
| A_23_P91504 | ITSN1 | c7 | AF064244 | 17405881 | 1.802 | 3.487 | 0.048 |
| A_24_P937256 | RELA | c8 | NM_001145138 | 9342183 | 0.688 | 1.611 | 0.039 |

| | | |
|---|---|---|
| Description | c1 | Homo sapiens catalase (CAT), mRNA [NM_001752] |
| | c2 | Homo sapiens suppressor of cytokine signaling 2 (SOCS2), mRNA [NM_003877] |
| | c3 | Homo sapiens Fas apoptotic inhibitory molecule (FAIM), transcript variant 1, mRNA |
| | | [NM_001033030] |
| | c4 | Homo sapiens heat shock 10 kDa protein 1 (chaperonin 10) (HSPE1), mRNA |
| | | [NM_002157] |
| | c5 | Homo sapiens HIV-1 Tat interactive protein 2, 30 kDa (HTATIP2), transcript variant 2, |
| | | mRNA [NM_006410] |
| | c6 | Homo sapiens tyrosine 3-monooxygenase/tryptophan 5-monooxygenase activation |
| | | protein, epsilon polypeptide (YWHAE), transcript variant 1, mRNA [NM_006761] |
| | c7 | intersectin 1 (SH3 domain protein) [Source: HGNC Symbol; Ace: 6183] |
| | | [ENST00000381318] |
| | c8 | Homo sapiens v-rel reticuloendotheliosis viral oncogene homolog A (avian) (RELA), |
| | | transcript variant 2, mRNA [NM_001145138] |

R*: Ratio to control (times)

(Cytokine or Chemokine-Associated Genes, Expression of Which was Significantly Reduced by EP)

TABLE 15

| ID | GeneSymbol | Description | GenbankAccession | log2 [ratio] | R* | p-value |
|---|---|---|---|---|---|---|
| Cytokines•chemokines | | | | | | |
| A_23_P259071 | AREG | g1 | NM_001657 | −2.969 | 0.128 | 0.000 |
| A_24_P389409 | BMP1 | g2 | NM_006129 | −0.622 | 0.650 | 0.002 |
| A_24_P60930 | BMP1 | g3 | NM_006129 | −0.706 | 0.613 | 0.001 |
| A_23_P131588 | BMP10 | g4 | NM_014482 | −0.678 | 0.625 | 0.043 |
| A_23_P68487 | BMP7 | g5 | NM_001719 | −2.883 | 0.136 | 0.007 |
| A_23_P135248 | CCL27 | g6 | NM_006664 | −0.846 | 0.556 | 0.028 |
| A_23_P407012 | CSF1 | g7 | NM_172210 | −0.753 | 0.593 | 0.008 |
| A_23_P45133 | CSF1 | g8 | M27087 | −0.732 | 0.602 | 0.006 |
| A_23_P213745 | CXCL14 | g9 | NM_004887 | −1.456 | 0.364 | 0.009 |
| A_23_P16523 | GDF15 | g10 | NM_004864 | −3.265 | 0.104 | 0.001 |
| A_23_P167585 | GDF9 | g11 | NM_005260 | −1.486 | 0.357 | 0.004 |
| A_23_P151294 | IFNG | g12 | NM_000619 | −0.710 | 0.611 | 0.031 |
| A_23_P67169 | IL11 | g13 | NM_000641 | −1.482 | 0.358 | 0.002 |
| A_23_P104798 | IL18 | g14 | NM_001562 | −0.641 | 0.641 | 0.007 |
| A_23_P72096 | IL1A | g15 | NM_000575 | −2.211 | 0.216 | 0.001 |
| A_23_P30122 | IL2 | g16 | NM_000586 | −0.898 | 0.537 | 0.011 |
| A_23_P76078 | IL23A | g17 | NM_016584 | −0.977 | 0.508 | 0.006 |
| A_24_P937317 | IL31 | g18 | NM_001014336 | −1.758 | 0.296 | 0.041 |
| A_23_P213706 | IL4 | g19 | NM_000589 | −1.251 | 0.420 | 0.024 |
| A_24_P209047 | IL5 | g20 | NM_000879 | −1.719 | 0.304 | 0.022 |
| A_23_P8961 | IL7 | g21 | NM_000880 | −3.273 | 0.103 | 0.020 |
| A_23_P127322 | NODAL | g22 | NM_018055 | −1.154 | 0.450 | 0.011 |
| A_23_P136493 | NRG1 | g23 | NM_013962 | −0.630 | 0.646 | 0.002 |
| A_23_P315815 | NRG1 | g24 | NM_004495 | −0.586 | 0.666 | 0.002 |
| A_23_P360777 | NRG1 | g25 | NM_013960 | −0.620 | 0.651 | 0.008 |
| A_23_P94754 | TNFSF15 | g26 | NM_005118 | −1.327 | 0.399 | 0.046 |
| A_23_P51361 | TXLNA | g27 | NM_175852 | −0.837 | 0.560 | 0.004 |
| A_23_P70398 | VEGFA | g28 | NM_001025370 | −0.997 | 0.501 | 0.004 |
| A_23_P81805 | VEGFA | g29 | NM_001025366 | −0.710 | 0.611 | 0.001 |
| A_24_P12401 | VEGFA | g30 | NM_001025366 | −0.889 | 0.540 | 0.002 |
| A_24_P179400 | VEGFA | g31 | NM_001025370 | −0.621 | 0.650 | 0.011 |

| | | |
|---|---|---|
| Description | g1 | Homo sapiens amphiregulin (AREG), mRNA [NM_001657] |
| | g2 | Homo sapiens bone morphogenetic protein 1 (BMP1), transcript variant BMP1-3, mRNA |
| | | [NM_006129] |
| | g3 | Homo sapiens bone morphogenetic protein 1 (BMP1), transcript variant BMP1-3, mRNA |
| | | [NM_006129] |
| | g4 | Homo sapiens bone morphogenetic protein 10 (BMP10), mRNA [NM_014482] |
| | g5 | Homo sapiens bone morphogenetic protein 7 (BMP7), mRNA [NM_001719] |
| | g6 | Homo sapiens chemokine (C-C motif) ligand 27 (CCL27), mRNA [NM_006664] |
| | g7 | Homo sapiens colony stimulating factor 1 (macrophage) (CSF1), transcript variant 2, mRNA |
| | | [NM_172210] |
| | g8 | Homo sapiens macrophage colony-stimulating factor (M-CSF1) cDNA to mRNA, complete |
| | | cds. [M27087] |
| | g9 | Homo sapiens chemokine (C-X-C motif) ligand 14 (CXCL14), mRNA [NM_004887] |
| | g10 | Homo sapiens growth differentiation factor 15 (GDF15), mRNA [NM_004864] |
| | g11 | Homo sapiens growth differentiation factor 9 (GDF9), mRNA [NM_005260] |
| | g12 | Homo sapiens interferon, gamma (IFNG), mRNA [NM_000619] |
| | g13 | Homo sapiens interleukin 11 (IL11), mRNA [NM_000641] |
| | g14 | Homo sapiens interleukin 18 (interferon-gamma-inducing factor) (IL18), mRNA |
| | g15 | Homo sapiens interleukin 1, alpha (IL1A), mRNA [NM_000575] |
| | g16 | Homo sapiens interleukin 2 (IL2), mRNA [NM_000586] |
| | g17 | Homo sapiens interleukin 23, alpha subunit p19 (IL23A), mRNA [NM_016584] |
| | g18 | Homo sapiens interleukin 31 (IL31), mRNA [NM_001014336] |
| | g19 | Homo sapiens interleukin 4 (IL4), transcript variant 1, mRNA [NM_000589] |
| | g20 | Homo sapiens interleukin 5 (colony-stimulating factor, eosinophil) (IL5), mRNA |
| | g21 | Homo sapiens interleukin 7 (IL7), mRNA [NM_000880] |
| | g22 | Homo sapiens nodal homolog (mouse) (NODAL), mRNA [NM_018055] |
| | g23 | Homo sapiens neuregulin 1 (NRG1), transcript variant GGF2, mRNA [NM_013962] |
| | g24 | Homo sapiens neuregulin 1 (NRG1), transcript variant HRG-gamma, mRNA [NM_004495] |
| | g25 | Homo sapiens neuregulin 1 (NRG1), transcript variant ndf43, mRNA [NM_013960] |
| | g26 | Homo sapiens tumor necrosis factor (ligand) superfamily, member 15 (TNFSF15), mRNA |
| | | [NM_005118] |
| | g27 | Homo sapiens taxilin alpha (TXLNA), mRNA [NM_175852] |
| | g28 | Homo sapiens vascular endothelial growth factor A (VEGFA), transcript variant 6, mRNA |
| | | [NM_001025370] |
| | g29 | Homo sapiens vascular endothelial growth factor A (VEGFA), transcript variant 1, mRNA |
| | | [NM_001025356] |
| | g30 | Homo sapiens vascular endothelial growth factor A (VEGFA), transcript variant 1, mRNA |
| | | [NM_001025366] |
| | g31 | Homo sapiens vascular endothelial growth factor A (VEGFA), transcript variant 6, mRNA |
| | | [NM_001025370] |

R*: Ratio to control (times)

(First Gene Group)

The first gene group consists of genes, expression of which is promoted by administration of EP to animal cells. Such genes include the genes shown in Table 9, Table 10 and Table 14. These genes are thought to be associated with the typical onset of action caused by EP. The first gene group is preferably the gene group shown in Table 9. It also preferably is comprised of the genes shown in Table 14.

(Second Gene Group)

The second gene group consists of genes, expression of which is suppressed by administration of EP to animal cells, and examples of such genes include the genes shown in Tables 11 to 13 and Table 15. These genes are thought to be associated with the typical onset of action caused by EP. The second gene group preferably is comprised of the genes shown in Tables 11 to 13. The second gene group also preferably is comprised of the genes shown in Table 15.

Expression of such genes can be confirmed by an expression analysis technique using an array or the like provided with probes for normal human total genome genes, or by expression analysis targeting specific genes. The array or the like and the necessary reagents may be obtained commercially, and comprehensive gene expression analysis and expression analysis of specific genes are well-known techniques that can be implemented as necessary by a person skilled in the art.

(Solid-Phase Carrier for Evaluation of Gene Expression Produced by EP)

The expression state of a specific gene as produced in animal cells by EP administration is preferably confirmed using a solid-phase carrier provided with a first region on which one or two or more probes for detecting one or two or more genes selected from the first gene group are fixed, and a second region on which one or two or more probes for detecting one or two or more genes selected from the second gene group are fixed.

With this solid-phase carrier, detection and determination are easy because the probes for detecting genes belonging to the first gene group and the probes for detecting genes belonging to the second gene group are retained at different positions on the carrier. A solid-phase carrier with such probes fixed thereon may be one for performing hybridization between the probes and a sample DNA by immersing the solid-phase carrier in a solution containing the sample DNA, or may be one for performing hybridization between the probes and a sample DNA by expanding such a solution on the solid-phase carrier. The material of the solid-phase carrier and the configuration of the fixed probes may be selected appropriately according to the form of hybridization. The hybridization product may be detected either using fluorescence, or visually with a visible label. Techniques of hybridization between probes that detect specific genes and DNA samples derived from RNA or DNA extracted from cells or the like are well known to those skilled in the art, and a person skilled in the art can prepare a solid-phase carrier for detecting the genes specified in the first gene group and second gene group based on known techniques.

This kind of solid-phase carrier can be applied not only to the expression methods disclosed in the present specification, but also to solid-phase carriers for screening regulators of EP action, methods for evaluating reactivity of animal cells to EP, methods of screening EP-like compounds and the like.

The expression of a specific gene group can be promoted and/or suppressed in this method by administering EP to animal cells. By obtaining animal cells with such expression, it is possible to create cells with enhanced cell functions, which can then be used to develop further functions and applications.

With the present method, EP can be administered to autologous animal cells (somatic cell, etc.), ES cells or iPS cells that are derived from a specific individual animal to achieve animal function enhancement (barrier function improvement, apoptosis suppression) or cell proliferation of the animal cells and obtain proliferative cells for autologous transplantation. These may also be differentiated as necessary. Such cells, and somatic cells and differentiated cells in particular, may also be cell structures with specific three-dimensional shapes (sheets, tubes or the like). The transplantation site is determined appropriately according to the state of the individual animal, and may be a deficient site or damaged site for example. Examples of autologous transplants include skin, cartilage, bone, neural tissue, liver and various other organs. These techniques are useful for proliferation and the like of cells that are to be used for various applications (fermentation, medical treatment (including prostheses and transplantation), drugs).

(Method for Evaluating Reactivity of Animal Cells to EP)

The method for evaluating reactivity of animal cells to EP that is disclosed in the present specification may comprise a step of administering EP to animal cells, a step of measuring an expression level of one or two or more genes selected from the first gene group and/or an expression level of one or two or more genes selected from the second gene group in the animal cells to which the EP was administered, and a step of evaluating the reactivity of the animal cells to EP based on the expression level of the one or two or more genes as obtained in the previous step.

By administering EP to animal cells in the present method, it is possible to evaluate the reactivity of EP with respect to those animal cells, and to select cells in which the proliferative function, barrier function or apoptosis suppression can be effectively enhanced by EP administration. The embodiments explained above with respect to the expression method can be applied as is to the animal cells, the administration of EP to animal cells, the first gene group and second gene group, and the detection of the same in this method.

Reactivity can be evaluated based on the number (ratio) of genes out of the genes belonging to the first gene group for which at least a certain increase in expression can be confirmed, and on the expression levels of the genes for which increased expression is confirmed. The higher the gene ratio or gene expression level, the more affirmatively the cells can be judged to have strong reactivity to EP. Conversely, the lower the gene ratio or gene expression level, the more affirmatively the cells can be judged to have low reactivity to EP.

Similarly, reactivity can be evaluated based on the number (ratio) of genes out of the genes belonging to the second gene group for which at least a certain reduction in expression can be confirmed, and on the expression levels of the genes for which reduced expression is confirmed. The lower the gene ratio or gene expression level, the more affirmatively the cells can be judged to have strong reactivity to EP. Conversely, the higher the gene ratio or gene expression level, the more affirmatively the cells can be judged to have low reactivity to EP.

(Method for Screening Compounds that Regulate the Action of EP)

The method disclosed in the present specification for screening compounds that regulate the action of EP may comprise a step of administering EP and one or two or more test compounds to animal cells, a step of measuring an expression level of one or two or more genes selected from the first gene group and/or an expression level of one or two or more genes selected from the second gene group in the animal cells, and a step of evaluating the suppressing action or enhancing action of the one or two or more test compounds on the action of EP based on the expression level of the one or two or more genes as obtained in the measurement step.

Compounds that further enhance or suppress the cell function enhancing action of EP can be screened by this screening method. Compounds that further enhance cell functions when used in combination with EP can be obtained in this way. Compounds that suppress the cell function enhancing action of EP when used in combination with EP can also be identified, and these compounds can be avoided. The embodiments explained above with respect to the expression method can be applied as is to the animal cells, the administration of EP to animal cells, the first gene group and second gene group and the detection of the same in this method.

A test compound may be a low-molecular-weight organic compound such as a hormone or cytokine or a lipid or other biological membrane component, or may be a high-molecular-weight organic compound such as a peptide, protein or sugar or DNA, RNA or a composite of these. Inorganic compounds or the like such as metal salts and various other inorganic salts are also possible. The method for administering the test compound and EP to animal cells is not particularly limited. Typically, animal cells can be cultured in medium containing the test compound and EP. The test compound can typically be supplied temporarily, continuously or intermittently to the animal cell medium.

The enhancing effect on the action of EP can be evaluated based on the number (ratio) of genes out of the genes belonging to the first gene group for which at least a certain increase in expression can be confirmed, and on the expression levels of the genes for which increased expression is confirmed. The greater the gene ratio or gene expression level, the more affirmatively the compound can be judged to have an enhancing effect on the action of EP. Conversely, the lower the gene ratio or gene expression level, the more affirmatively the compound can be judged to have a suppressing effect on the action of EP.

Similarly, the enhancing effect on the action of EP can be evaluated based on the number (ratio) of genes out of the genes belonging to the second gene group for which at least a certain reduction in expression can be confirmed, and on the expression levels of the genes for which reduced expression is confirmed. The lower the gene ratio or expression level, the more affirmatively a compound can be judged to have an enhancing effect on the action of EP. Conversely, the higher the gene ratio or expression level, the more affirmatively the compound can be judged to have a suppressing effect on the action of EP.

(Method for Screening EP-Like Compounds)

The method for screening EP-like compounds disclosed in the present specification may comprise a step of administering, to animal cells, one or two or more test compounds selected from the group consisting of expression products of one or two or more genes selected from the first gene group, compounds that promote expression of these genes, compounds that act suppressively on expression products of one or two or more genes selected from the second gene group, and compounds that suppress expression of these genes, and a step of measuring one or two or more cell functions of the animal cells.

With this screening method, a compound that exhibits at least some of the actions of EP can be efficiently screened by selecting and administering, to cells, test compounds selected from the group consisting of the expression products of genes associated with the action of EP, the compounds that promote expression of such genes, the compounds that act suppressively on the expression products and the compounds that suppress gene expression, and then evaluating cell function.

Examples of gene expression products include RNA and proteins. Examples of compounds that promote expression include transcription factors and compounds that activate transcription factors and the like. Examples of compounds that act suppressively on expression products include siRNA that causes mRNA expression products to be broken down, and proteins and the like that compete with protein expression products. In addition to siRNA, other examples of compounds that suppress gene expression include knockout constructs and the like. The method for administering such a compound can be determined appropriately according to the type of compound. In the case of a protein for example, the protein must be administered in the form of a protein expression construct for introduction into cells. In the case of siRNA, a siRNA construct may be introduced into cells, or the siRNA itself may be introduced into cells. A low-molecular-weight organic compound or other compound that passes through animal cell membranes may be added to medium.

Cell functions can be evaluated based on the functions obtained when EP is used as an animal cell function enhancer as explained above. That is, the action can be evaluated in various ways depending on the type of cells, the type of test compound and the type of action, and this can be determined appropriately by a person skilled in the art according to the purpose of evaluation.

(Cytokine Suppressor and Anti-Inflammatory Agent)

EP can also be used as a cytokine suppressor. Suppression of the expression of genes coding for cytokines and chemokines by administration of EP to cells has been confirmed. Cytokines and chemokines, expression of which has been reduced by EP administration, were shown already in Table 15. These cytokines and chemokines are all inflammatory cytokines. As also shown in the examples below, induction of IL-8 and TNF-α was clearly suppressed in animal cells that had been subjected to LPS stimulus. Thus, EP is a suppressor of cytokines and particularly inflammatory cytokines. Examples of inflammatory cytokines (inflammation-inducing cytokines) include AREG, BMP7, CCL27, CSF1, CXCL14, GDF15, IL11, IL18, IL23A, IL31, IL4, IL5, TNFSF15, VEGFA and the like.

EP can also be used as an anti-inflammatory agent. That is, EP can be used in the form of a composition for prevention or treatment of inflammatory conditions caused by inflammation reactions. As described above, EP suppresses expression of genes coding for inflammatory cytokines, and suppresses induction of the inflammatory cytokines IL-8 and TNF-α in animal cells that have been subjected to LPS stimulus (method of Singh et al.). These anti-inflammatory properties of EP were evaluated using THP-1, an established human monocytic cultured cell line that has long been used in research as a macrophage model. It has recently been confirmed that this cell line is also useful in h-CLAT testing for evaluating allergens (Toxicol. In Vitro, 2006 August; 20(5): 763-73). Singh et al. (Clin. Chem. 2005 December 51(12): 2252-6) have also shown that this cell line can be applied to evaluating compounds and foods with anti-inflammatory effects without using animal subjects.

Examples of inflammatory conditions that can be treated with EP include, but are not limited to: autoimmune conditions affecting multiple organs (systemic lupus erythematosus (SLE), scleroderma and the like for example), specific tissues or organs (such as musculoskeletal tissue (rheumatoid arthritis, ankylosing spondylitis)), the digestive tract (Crohn's disease, ulcerative colitis), the central nervous system (Alzheimer's disease, multiple sclerosis, motor neuron disease, Parkinson's disease, chronic fatigue syndrome), the beta cells of the pancreas (insulin-dependent diabetes), the adrenal gland (Addison's disease), the kidneys (Goodpasture's syndrome, IgA nephropathy, interstitial nephritis), the exocrine glands (Sjogren's syndrome, autoimmune pancreatitis) and the skin (psoriasis, atopic dermatitis); chronic inflammatory conditions (for example, osteoarthritis, periodontal disease, diabetic nephropathy, diabetic ulcer, retinopathy, chronic obstructive pulmonary disease, arteriosclerosis, graft-versus-host disease, chronic pelvic inflammatory disease, endometriosis, chronic hepatitis, tuberculosis and the like); and IgE mediated (type I) hypersensitivity (for example, rhinitis, asthma, anaphylaxis, dermatitis, eye disease and the like). Pathologies of dermatitis include actinic keratosis, acne rosacea, acne cornedo, allergic contact dermatitis, angioedema, atopic dermatitis, bullous pemphigoid, skin drug reactions, erythema multiforme, lupus erythematosus, photodermatitis, psoriasis, psoriatic arthritis, scleroderma and rash. Eye symptoms include age-related macular degeneration (ARMD), dry eye, uveitis and glaucoma.

EP may be used according to the present invention in cases in which other treatment drugs selected from the known anti-inflammatory drugs, antibiotics and the like used for inflammatory disease would be administered, and may also be administered in combination with these other treatment drugs.

When EP is used in the form of a composition for preventing or treating inflammatory diseases, any appropriate administration route may be used. For example, a peroral, topical, parenteral, intraocular, rectal, vaginal, inhalatory, intraoral, sublingual, or intranasal delivery route may be appropriate. A suitable pharmaceutical composition may be used for this purpose.

A pharmaceutical composition containing an active ingredient may be in a form suited to oral use, such as a tablet, pastille, lozenge, aqueous or oil-based suspension, dispersible powder or granules, emulsion, hard or soft capsule, syrup or elixir. The composition may also be in a prompt release form or a controlled release form.

A composition intended for oral use may be prepared according to any method known to a person skilled in the art for manufacturing pharmaceutical compositions, and such a composition may also contain one or more substances selected from the group consisting of the sweeteners, flavoring agents, colorants and preservatives, which can be combined and used appropriately by a person skilled in the art.

In addition, a person skilled in the art may select other appropriate additives as necessary when manufacturing an aqueous suspension, oil-based suspension, powder or granules, emulsion, hard or soft capsule, syrup or elixir or other known drug formulation containing EP as an active ingredient.

EP may also be formulated as a suppository, or as a cream, ointment, jelly, solution or suspension for topical use or the like. Topical applications include mouthwashes and oral rinses.

The effective dosage of EP differs depending on the individual to be treated and the administration method. For example, in the case of a preparation for oral administration in humans it may vary between 1% and 99% of the total amount of the composition. For example, a dosage unit form normally contains about 1 mg to 1 mg of the active ingredient. It can be understood that the administration level of EP in any individual depends on a variety of factors including the individual's age, weight, general state of health, gender and diet time of administration and on the administration route, excretion rate, combination of drugs, and severity of the specific symptoms to be treated.

(Antioxidant or Anti-Oxidative Stress Agent)

EP also increases the resistance of cells to oxidation. Thus, EP can be used as an anti-oxidative stress agent. That is, it can be used as an agent for treating or preventing conditions caused by oxidation in vivo. Considering that increased expression of the antioxidant substances thioredoxin (TXN) and hydrogen peroxide degrading enzyme (CAT) is found when EP is administered to cells, and as explained in the examples below, it appears that EP reduces cytotoxicity caused by hydrogen peroxide. Thus, EP can confer resistance to oxidative stress on cells.

Conditions caused by oxidation and oxidative stress include atherosclerotic disease and other forms of arteriosclerosis and angina associated with arteriosclerosis, Parkinson's disease, angina, myocardial infarction, Alzheimer's disease, amyotrophic lateral sclerosis, cerebral infarction, schizophrenia, bipolar disorder, fragile X syndrome, chronic fatigue syndrome, hyperlipidemia, diabetes, hypertension, cardiac arrest and liver cancer, as well as the inflammatory conditions described above for example.

EP can be used in the form of a food or nutritional supplement composition, or as an additive to these for achieving various functions. The food is not particularly limited, and examples include various solid foods (including gel foods) and various liquid foods.

EP can be prepared using any suitable method known in the technical field. That is, EP can be obtained commercially, and can also be obtained easily by a person skilled in the field of organic synthetic chemistry from commercially available substances.

All of the documents of prior art cited in the present specification are incorporated by reference into the present specification.

### EXAMPLES

The disclosures of the present specification are explained below using concrete examples, but the examples below do not limit the disclosures of the present specification.

### Example 1

(Cell Proliferation Effect of Ethanolamine Phosphate)

Cells of an established line of normal human airway epithelial cells (BEAS-2B cells) (ATCC) were seeded to 1×10⁴ cells/cm² on a 96-well plate. 10% FBS RPMI-1640 was used as the medium. After 24 hours, the cells were washed gently with 200 μl/well of PBS, and the medium was replaced with 0%, 0.125%, 0.25% and 0.5% FBS (90 μl well). Following medium replacement, 10 μl/well of EP solution was added. A proliferation test (WST-8 assay) was performed after 96 hours. The operations of the WST-8 assay were as follows.

(1) The medium was removed from the 96-well plate by decantation and tapping.

(2) WST-8 reagent (Dojindo) was mixed to a ratio of 1:10 with serum-free medium (RPMI-1640) that had been warmed in advance at 37° C., and added to the wells from which the medium had been removed.

(3) A color reaction was performed for 1 hour in a CO₂ incubator, and absorbance at 450 nm and 650 nm was measured with an absorbance plate reader.

(4) Absorbance at 450 nm—absorbance at 650 nm was calculated for purposes of correction between plates.

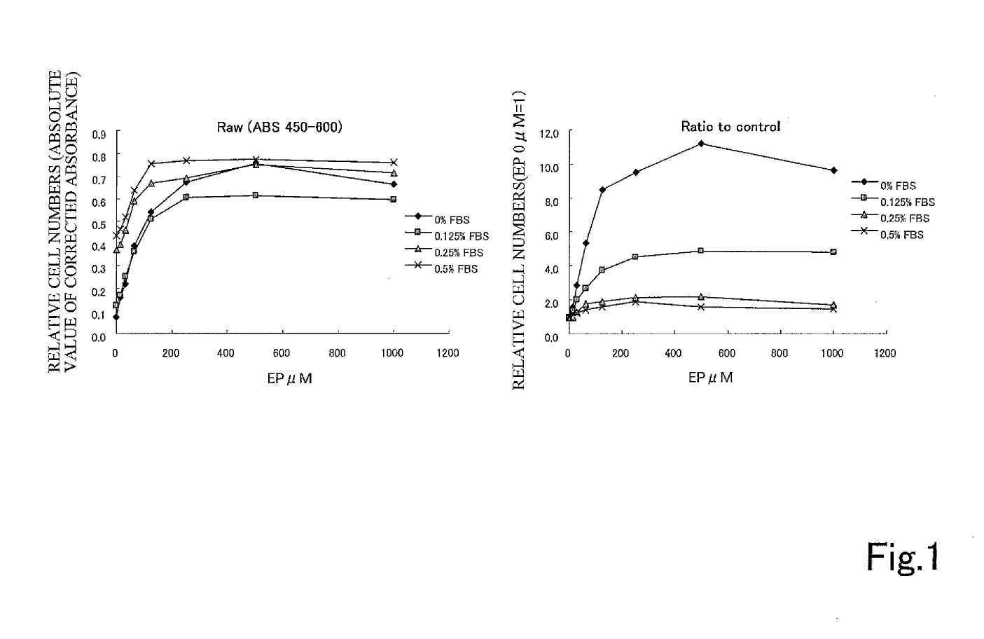

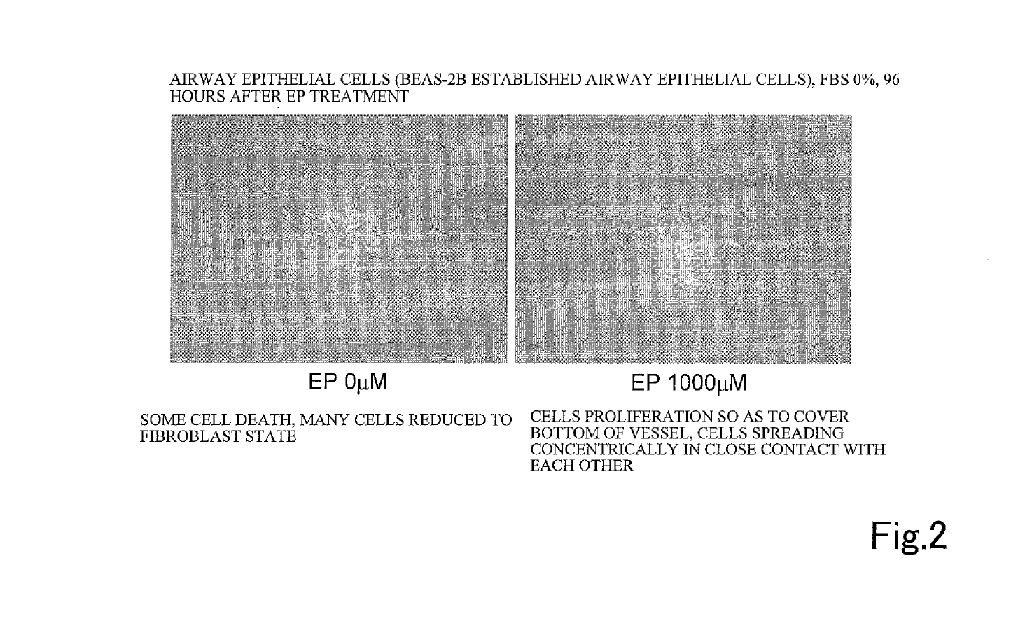

(5) The average of the 4 blank wells was subtracted from the corrected value of all wells, and the result was given as the relative corresponding cell number. The results are shown in FIG. 1 and FIG. 2.

FIGS. 1A and 1B show cell proliferation results using EP in BEAS-2B cells at different FBS concentrations. FIG. 1A shows the absolute value, while FIG. 1B shows the ratio relative to a control using 0 μM EP.

As shown in FIG. 1A, proliferation of BEAS-2B cells was dependent on EP concentration. Even at the high-concentration range (mM level), there was no obvious toxicity attributable to EP. Moreover, EP caused cell proliferation even under conditions with no FBS at all, and the resulting cell proliferation that was not that much different from that obtained with 0.5% FBS. As shown in FIG. 1B, when the medium contains no FBS the cells died due to low-serum stress (see EP 0 μM). However, when EP was added cell proliferation was as high as 11 times the control value (FIG. 1B).

FIG. 2 shows images of cell proliferation with EP 0 μM and EP 1000 μM. As shown in FIG. 2, at EP 0 μM cells died or were reduced to a fibroblast-like shape, while at EP 1000 μM the cells proliferated so as to cover the bottom of the vessel. The cells also spread concentrically in close contact with each other.

### Example 2

(Proliferative Effect in Primary Normal Human Airway Epithelial Cells (HBEpC))

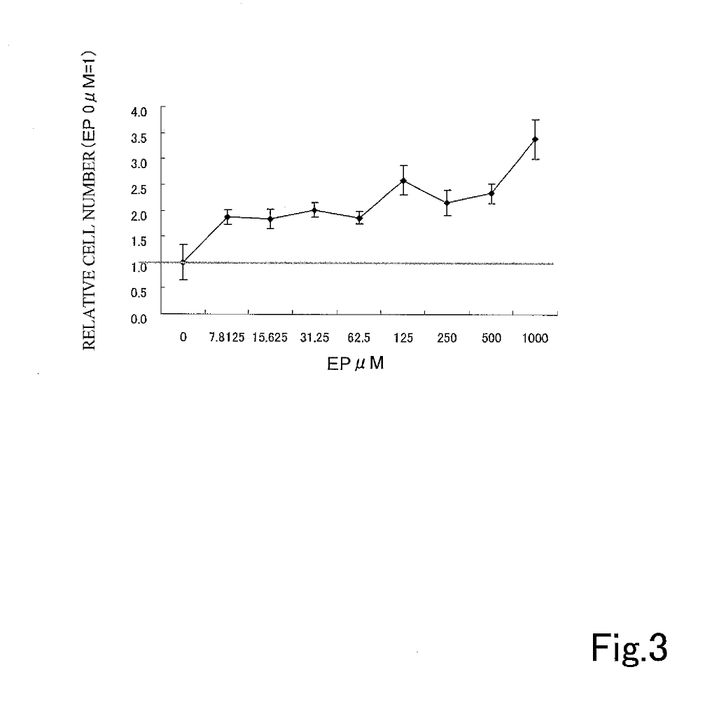

HBEpC cells (Iwaki) were seeded to 3×10⁴ cells/cm² on a 96-well plate. BEGM (Takara Bio, complete medium) was used as the medium. After 24 hours, the cells were washed gently with 200 μl per well of HEPES buffer, and the medium was replaced with BEBM basal medium (medium containing no pituitary extract or peptide growth factor) with only the antibiotic GA-1000 added thereto (90 μl per well). Following medium replacement, 10 μl per well of an EP solution of 10 times the set concentration was added, and after 96 hours a proliferation test (WST-8 assay) was performed. The WST-8 assay was performed as in Example 1. The results are shown in FIG. 3.

As shown in FIG. 3, EP also had a growth-stimulating effect on primary normal human airway epithelial cells. Because the basal medium contained no pituitary extract or peptide growth factor, the growth-stimulating effect on primary normal human airway epithelial cells was obtained in serum-free medium without relying on the combined or synergistic effects of these. The growth-stimulating effect of EP is also dose-dependent, with no toxicity even at the mM level and a roughly two-fold growth-stimulating effect was even at 7 μM.

### Example 3

(Proliferative Effect in Primary Normal Human Lung Fibroblasts (NHLF))

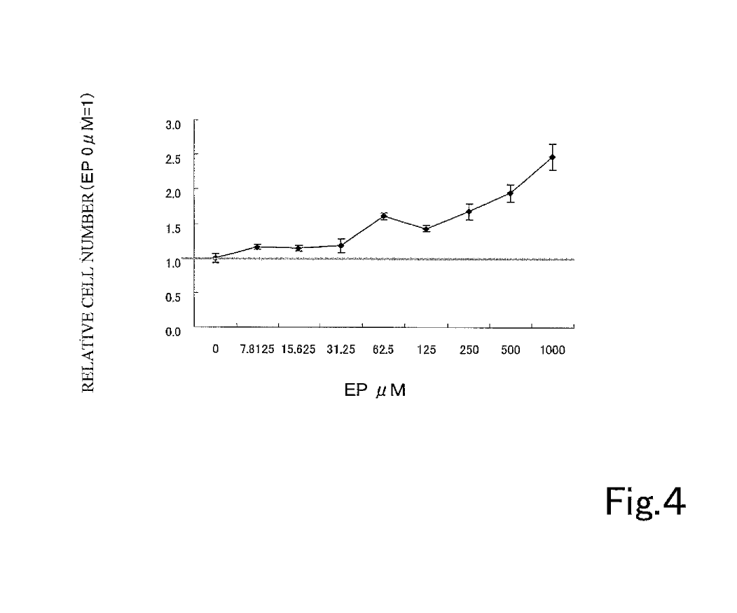

NHLF cells (Takara Bio) were seeded to 3×10⁴ cells/cm² on a 96-well plate. 10% FBS-DMEM was used as the medium. After 24 hours, the cells were washed gently with 200 μl per well of PBS, and the medium was replaced with 0% FBS DMEM (90 μl per well). Following medium replacement, 10 μl/well of EP solution was added, and a proliferation test (WST-8 assay) was performed after 124 hours. The WST-8 assay was performed as in Example 1. The results are shown in FIG. 4.

As shown in FIG. 4, EP was confirmed to have a growth-stimulating effect even in primary human fibroblasts. Proliferation of lung fibroblasts plays a major role in airway remodeling in asthma patients. In the past it was thought that airway remodeling contributed to the pathologies of asthma and chronic obstructive pulmonary disease, but it is becoming clear that proliferation of fibroblasts plays an important role in the wound healing process of injured epithelium, so this is an example healing effect of EP against asthma.

### Example 4

(Proliferative Effect in Primary Normal Keratinocytes (HEKa)

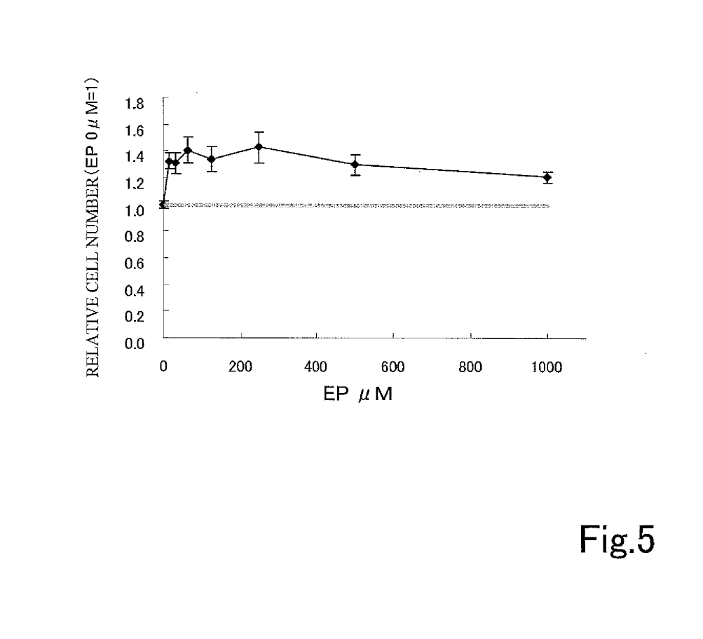

HEKa cells (Takara Bio) were seeded to 2×10⁴ cells/cm² on a 48-well plate. Epilife basal medium (M-EPI-500-CA, Invitrogen) with HKGS Kit (S-001-K, Invitrogen) as an added factor was used as the medium. This HKGS Kit contains bovine pituitary extract, bovine insulin, hydrocortisone, bovine transferrin and human EGF. After 24 hours, the cells were washed gently with 400 μl per well of PBS, the medium was replaced with 252 μl per well of Epilife basal medium, and the cells were cultured for 24 hours. After culture, 28 μl per well of EP solution was added, and a proliferation test (WST-8 assay) was performed 21 days after EP administration. The WST-8 assay was performed as in Example 1 except with 2.8 times the amounts of reagent and medium. The results are shown in FIG. 5.

As shown in FIG. 5, EP had a growth-stimulating effect even in primary human keratinocytes. Roughly the maximum growth-stimulating effect was obtained at a minimum concentration of 15.6 μM. Keratinocytes are skin-forming cells, so substances that enhance the growth and functions of these cells are extremely useful. EP can be applied during preparation of skin sheets in regenerative medicine, and can be used as a medium component for preparing tissue sections containing no animal proteins and having few allergic effects.

### Example 5

(Proliferative Effect in Primary Normal Human Hair Dermal Papilla Cells (HHDPC))

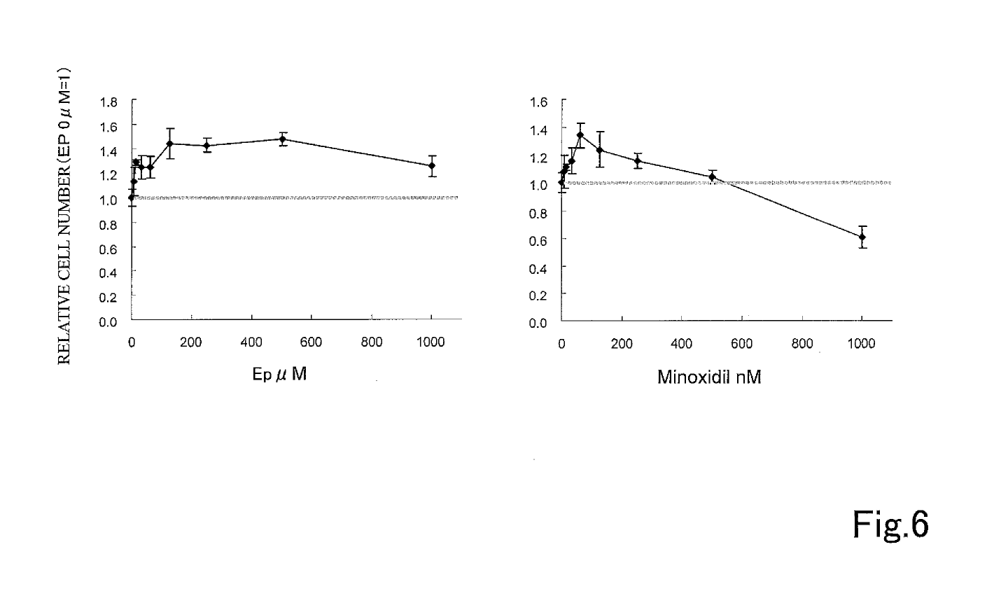

HHDPC cells (Cosmo Bio) were seeded to 1×10⁴ cells/cm² on a 96-well plate. Complete MSCM medium (500, ScienCell) was used as the medium. After 24 hours, the cells were washed gently with 200 μl per well of PBS, the medium was replaced with 90 μl per well of 0% FBS DMEM with 1 vol % of complete MSCM medium added thereto, and the cells were cultured for 24 hours. After culture, 10 μl per well of EP solution was added, and a proliferation test (WST-8 assay) was performed 6 days after EP administration. The WST-8 assay was performed as in Example 1. The same test was performed using minoxidil, which has a clinical hair growth effect. The results are shown in FIG. 6.

As shown in FIG. 6, EP had a proliferative effect equal to or greater than that of minoxidil, which is known to have a clinical hair growth effect. Minoxidil has a rather narrow effective concentration range of tens of nM to 400 nM, and is toxic above that range, but the present component exhibited proliferative effects within a very broad concentration range, and has been shown to be highly safe. Moreover, although monixidil exhibits cell toxicity at high concentrations, EP is useful because it is an endogenous cell substance and non-toxic. A correlation has already been found between proliferative effects in HHDPC cells and hair-growth effects, so clearly EP could be used as a hair growth or hair restoration agent.

### Example 6

(Proliferative Effects in Cancer Cells and Immortalized Model Cells)

(1) A549 and HEK293 Cells; Attachment Cells

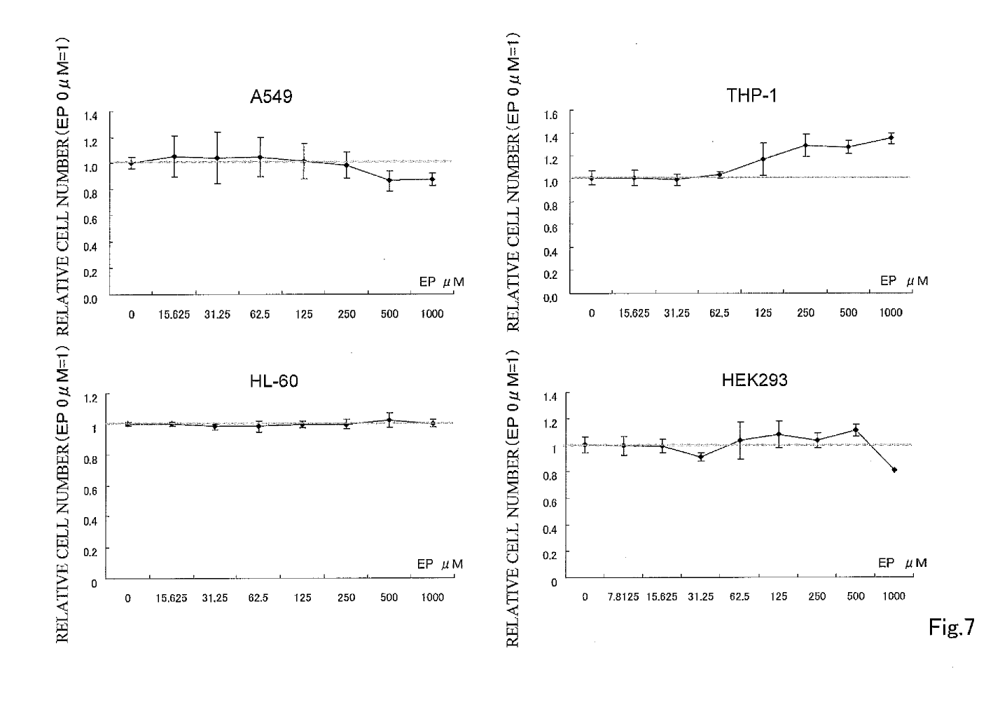

A549 (human type II alveolar epithelial cells: adenocarcinoma) and HEK 293 (adenovirus immortalized human embryonic kidney cells) were seeded to 1×10⁴ cells/cm² on a 96-well plate. 0% FBS RPMI-1640 was used as the medium. 24 hours after seeding the cells were washed with 20 μl per well of PBS, and the medium was replaced with 0% FBS RPMI-1640. Following medium replacement, EP solution with 10 times the set concentration was added 10 μl per well, and a proliferation test (WST-8 assay) was performed 96 hours after addition of EP. The WST-8 assay was performed as in Example 1. The results are shown in FIG. 7.

(2) THP-1, HL-60 Cells; Floating Cells

THP-1 (human acute monocytic leukemia cells) and HL-60 (human myeloid leukemia cells) were logarithmically growth cultured in 10% FBS RPMI-1640 to 2 to 10×10⁵ cells/cm² to achieve logarithmic growth before EP administration. The cell culture liquid was then centrifuged and precipitated at 1000 rpm, and 90 μl per well was dispensed onto a 96-well plate to 2×10⁴ cells/cm² in 0% FBS RPMI-1640. 10 μl per well of EP solution was added, and a proliferation test (WST-8 assay) was performed after 72 hours. For the WST-8 assay of the floating cells, 10 μl per well of WST-8 assay reagent was added directly to the cell suspension during culture, and after 1 hour absorbance at 450 nm and 600 nm was measured with a plate reader and the data were analyzed as in the examples above. The results are shown in FIG. 7.

As shown in FIG. 7, EP exhibited absolutely no cell proliferation ability even at high concentrations with A549, HL60 and HEK 293. With THP-1, on the other hand, a small cell proliferation effect was observed at high concentrations of 125 μM or more.

These results show that EP has a highly selective proliferative effect on normal cells, but has almost no proliferative effect on cancer cells.

### Example 7

(Test Confirming Suppression of the Proliferative Effect of this Component by a MEK 1/2 Inhibitor)

Regulation of cell proliferation is mediated by a number of intercellular signals. In general, a growth signal (hormone) from outside the cell binds to a cell membrane receptor, activating proteins inside the cell by a hierarchical signaling cascade, at the end of which a mitogen-activated protein kinase (MAPK) moves inside the cell nucleus, regulating expression of a gene necessary for cell proliferation. Several families of MAPKs are known, of which ERK 1/2, JNK, p38, ERK5, ERK7 and the like have been identified. Any of the mechanisms described above may be responsible for the proliferative effect of EP. Preliminary investigation has shown that the proliferative effect of EP is suppressed by inhibitors of MEK 1/2, a molecule upstream from ERK 1/2 that activates (phosphorylates) ERK 1/2.

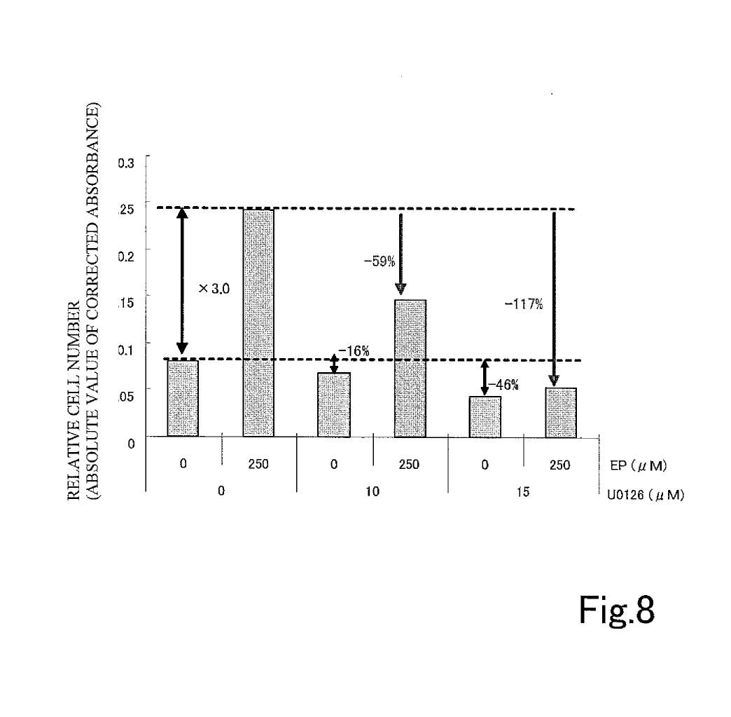

Cells of the established normal human airway epithelial cell line BEAS-2B were seeded to 1×10⁴ cells/cm² on a 96-well plate. 10% FBS RPMI-1640 was used as the medium. After 24 hours, the medium was replaced with 90 μl per well of 0.1% FBS RPMI-1640 containing the MEK 1/2 inhibitor U0126 (Cayman). 10 μl/well of a 2.5 μM EP solution was then added (final concentration 250 nM). A proliferation test (WST-8 assay) was performed after 72 hours. The WST-8 assay was performed as in Example 1. The results are shown in FIG. 8.

As shown in FIG. 8, the cells proliferated roughly 3-fold after treatment with 250 μM EP when no MEK 1/2 inhibitor was added. When a different MEK 1/2 inhibitor was added, on the other hand, a decrease in cell numbers was observed from treatment with the inhibitor alone, but the anti-proliferative effect with 25 μM of EP was even greater. This shows that one proliferative mechanism of EP involves growth signaling via MEK 1/2 and ERK 1/2, a MAPK downstream from MEK 1/2.

### Example 8

(Proliferative Effects of Substances Metabolically Upstream and Downstream from EP)

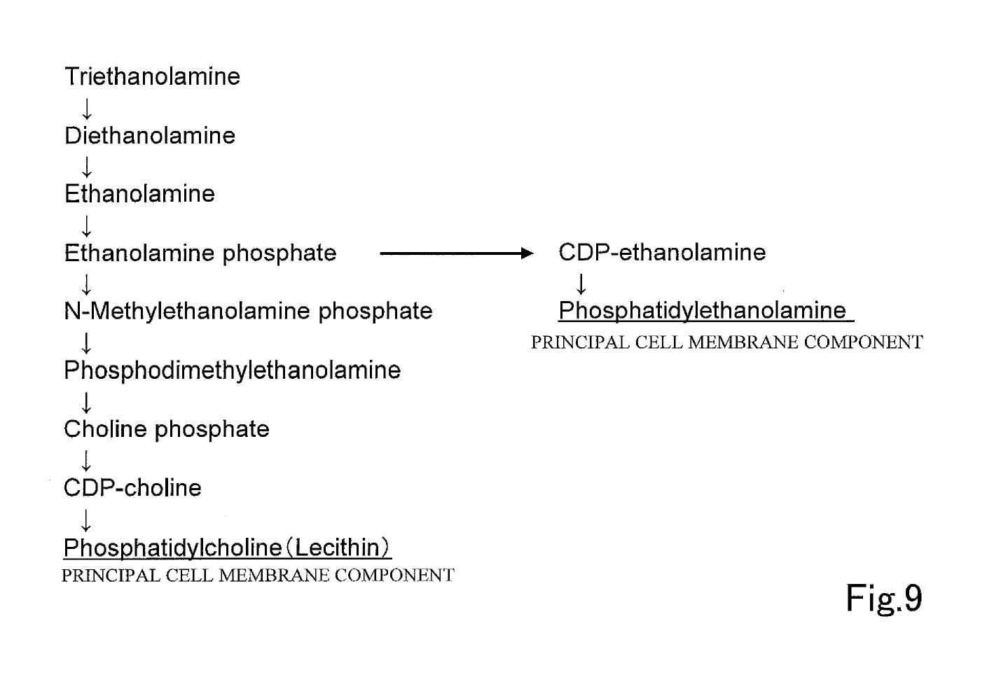

EP is known as a precursor of phospholipids essential for cells, such as phosphatidylcholine (lecithin), which is a key component of cell membranes, and phosphatidylethanolamine, which is common in neural tissue. As an experiment to test whether the proliferative effects of EP were due to replenishment of these cell constituents, we tested the proliferative effects of substances upstream and downstream from EP in the metabolic pathway. FIG. 9 shows a metabolic map of EP and components upstream and downstream from EP leading to these cell constituents.

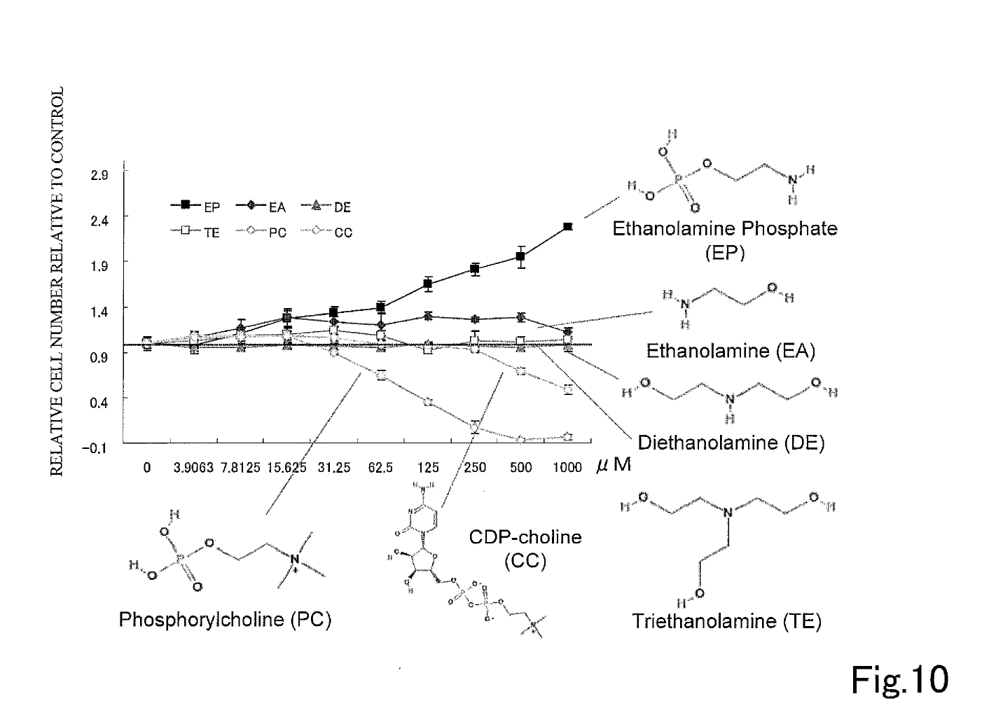

Cells of the established normal human airway epithelial cell line BEAS-2B were seeded to 1×10⁴ cells/cm² on a 96-well plate. 10% FBS RPMI-1640 was used as the medium. After 24 hours, the medium was replaced with 90 μl per well of 0.1% FBS RPMI-1640. EP and metabolic derivatives of EP were added 10 μl/well. A proliferation test (WST-8 assay) was performed after 72 hours. The WST-8 assay was performed as in Example 1. The proliferative and toxic actions of EP and its derivatives are shown in FIG. 10.

As shown in FIG. 10, while EP has a dose-dependent proliferative action, no great growth-promoting action was seen with the other components. The action of ethanolamine, which is metabolically upstream from EP, was shown to be much weaker than that of EP. These results show that the proliferative action of EP is a characteristic action of EP, and is not attributable to replenishment of lipid precursors.

### Example 9

(Proliferative Effect of GABA Receptor Agonist and Anti-Proliferative Effect of GABA-A Receptor Antagonist)

Homotaurine, a receptor agonist for the biologically active substance GABA-A, was found in a search for structural analogs of the present component in the comprehensive chemical database PubChem (http://pubchem.ncbi.nlm.nih.gov/). An intervention test was performed with GABA agonists and an antagonist to confirm whether the cell proliferative effects of EP are mediated by the GABA receptor.

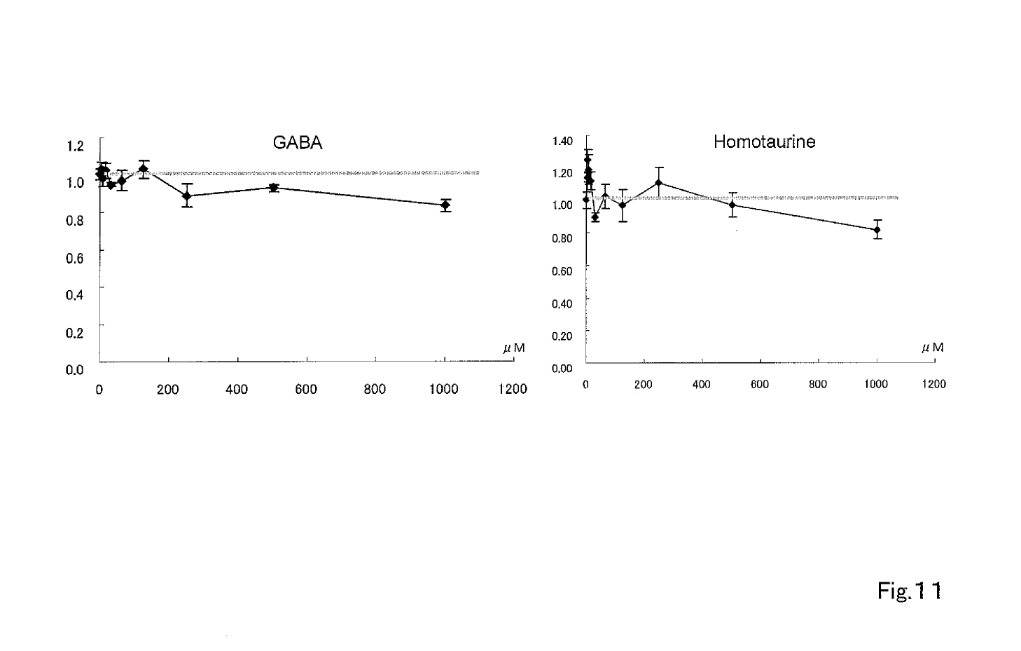

Cells of the established normal human airway epithelial cell line BEAS-2B were seeded to 1×10⁴ cells/cm² on a 96-well plate. 10% FBS RPMI-1640 was used as the medium. After 24 hours, the medium was replaced with 90 μl per well of 0.1% FBS RPMI-1640. Next, 10 μl per well of GABA (GABA-A and GABA-B receptor agonist) and homotaurine (GABA-A agonist) were added. A proliferation test (WST-8 assay) was performed after 72 hours. The WST-8 assay was performed as in Example 1. The results are shown in FIG. 11.

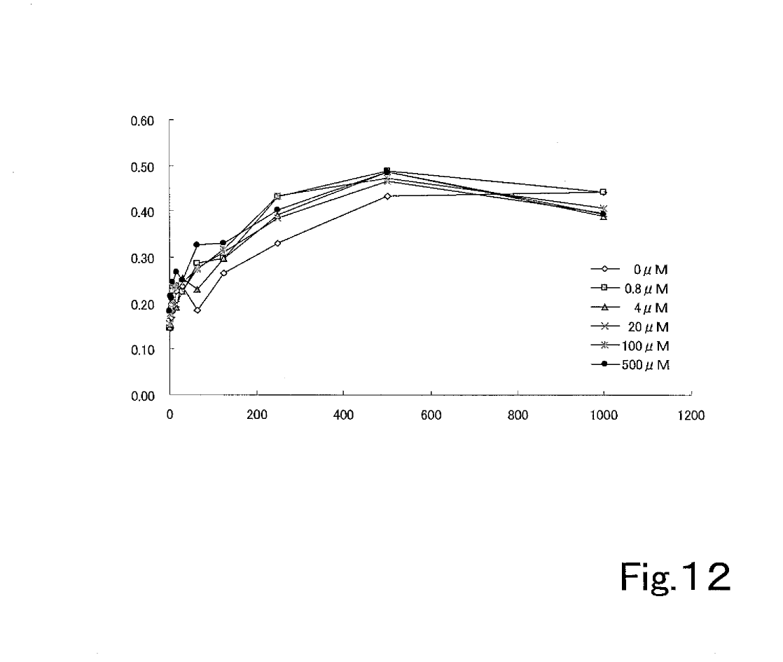

Cells of the established normal human airway epithelial cell line BEAS-2B were seeded to 1×10⁴ cells/cm² on a 96-well plate. 10% FBS RPMI-1640 was used as the medium. After 24 hours, the medium was replaced with 90 μl per well of 0.1% FBS RPMI-1640 containing a GABA-A inhibitor ((−)-Bicuculline Methochloride, #14343 Sigma; abbreviated as BM)). 10 μl per well of EP solution was added. A proliferation test (WST-8 assay) was performed after 72 hours. The WST-8 assay was performed as in Example 1. The results are shown in FIG. 12.

As shown in FIG. 11, neither the GABA-A and GABA-B receptor agonist GABA nor the EP structural analog and GABA-A receptor agonist homotaurine exhibited any statistically significant proliferative action. As shown in FIG. 12, moreover, the proliferative action of EP was not inhibited by the GABA-A receptor antagonist BM. This shows that the proliferative action of EP is produced by a mechanism that does not involve the GABA receptor.

### Example 10

(Inhibitory Effect of EP on Serum-Free Stress-Induced Elevation of Caspase 3/7 Activity)

EP by itself causes cell proliferation without serum. In general, animal cells cannot survive without the presence of multiple growth factors. When these are lacking, a self-destruct function of cells called apoptosis (programmed cell death) operates. Some of the intracellular pathways of apoptosis are known, and all of the known apoptosis pathways are associated with increased activity of a peptidase called caspase 3/7. One way to assess whether the present component has an apoptosis regulating action is by measuring caspase 3/7 activity. The following test was performed for this reason.

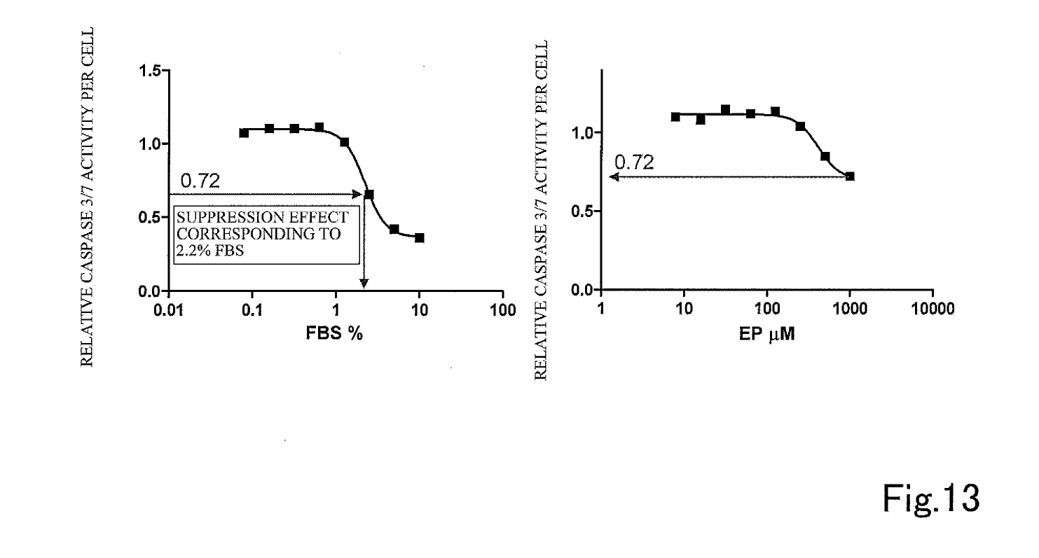

Cells of the established normal human airway epithelial cell line BEAS-2B were seeded to 5×10⁴ cells/cm² on a 96-well plate. 10% FBS RPMI-1640 was used as the medium. After 24 hours, the cells were washed gently with 200 μl per well of PBS, and the medium was replaced with 90 μl per well of 0% FBS RPMI-1640. After medium replacement, 10 μl per well of EP or FBS solution was added. After 24 hours, a proliferation test (WST-8 assay) and caspase 3/7 assay (Caspase-Glo3/7 assay, Promega) were performed. The WST-8 assay was performed as in Example 1, while the caspase 3/7 assay was performed according to the attached protocols. Caspase 3/7 activity was measured by a luminescence test, the blank average was subtracted, and this was divided by the corrected absorbance (corresponding to relative cell number) from the WST-8 assay to calculate the caspase 3/7 activity per cell. The results are shown in FIG. 13.

As shown by the relationship between FBS concentration and caspase 3/7 activity on the left side of FIG. 13, caspase 3/7 activity that was increased by substitution of serum-free medium was reduced dose-dependently by addition of FBS. As shown by the relationship between EP concentration and caspase 3/7 activity on the right side of FIG. 13, meanwhile, caspase 3/7 activity was reduced to about 0.72-fold by 1000 μM of EP. According to the regression curve, EP 1000 μM corresponds to the effect of FBS 2.2%. This shows that in addition to its cell proliferation function, EP strongly inhibits caspase 3/7, which represents an endpoint in the signaling pathway of apoptosis. This shows that EP functions as an apoptosis suppressor.

### Example 11

(Barrier Functions Produced by EP (Transepithelial Electrical Resistance, Substance Permeability) in an Established Line of Normal Airway Epithelial Cells)

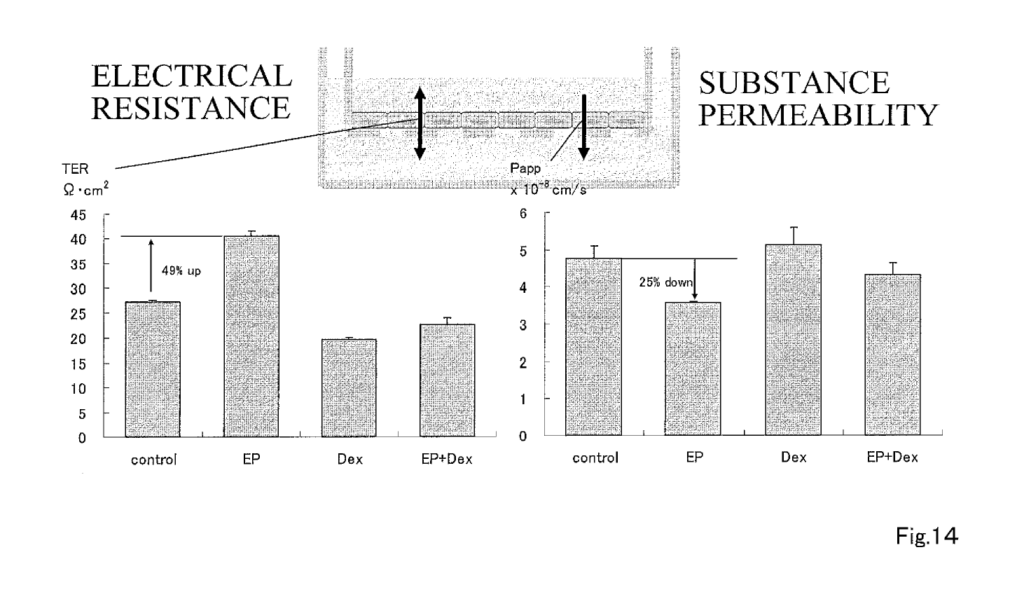

Cells of the established normal human airway epithelial cell line BEAS-2B were seeded to 1×10⁵ cells/cm² on a cell culture insert (353104, BD Falcon) (FIG. 14). 10% FBS RPMI-1640 was used as the medium. The amount of medium was 200 μl on the insert side and 750 μl on the well side. Beginning the next day, substitution/medium replacement was performed once a day with 1% FBS RPMI-1640 prepared with the following 4 reagent conditions, and transepithelial electrical resistance (TER) was measured with MilliCell (Millipore).
Condition 1: Control
Condition 2: EP 1 mM
Condition 3: Dexamethasone 1 μM (dexamethasone (Dex): positive control substance known to enhance barrier function)
Condition 4: EP 1 mM and Dex 1 μM

7 days after cell seeding a permeability test was performed as follows.

### Permeability Test

A 4-kilodalton fluorescent dextran molecule (Sigma) was added to the cell culture insert to a concentration of 0.1 mg/ml, 20 μl of the medium on the well side was removed after 1 hour, and the amount of permeating fluorescent dextran was estimated from the fluorescent strength at each concentration. The permeation rate Papp of a substance from the insert side to the well side is normally determined by the following formula. The results are shown in FIG. 14.

Papp=(volume of medium on well side/insert area×initial insert concentration)×(altered concentration on well side/elapsed time)

As shown on the left side of FIG. 14, a 49% increase in TER was seen in the normal human airway epithelial BEAS-2B cells to which EP was administered. The epithelial barrier function is known to correlate generally with transepithelial electrical resistance (TER), and the substance permeation rate is inversely correlated with TER. As shown on the right side of FIG. 14, when the permeation rate Papp of the fluorescent dextran molecule was measured, the permeation rate was 25% lower, or in other words the barrier function was greater in the cells to which EP was administered. In these cells, no rise in TER or drop in Papp was observed from treatment with the positive control substance dexamethasone alone or from treatment with both dexamethasone and EP. Thus, it appears that because EP promotes barrier function and has a proliferative effect on epithelial cells, it could be used to treat asthma by blocking permeation of allergens and the like in the damaged airway regions of asthma patients.

### Example 12

(Barrier Function Modification Action of EP in Cells Other than BEAS-2B)

(Calu-3 Human Cancer Airway Epithelial Cells)

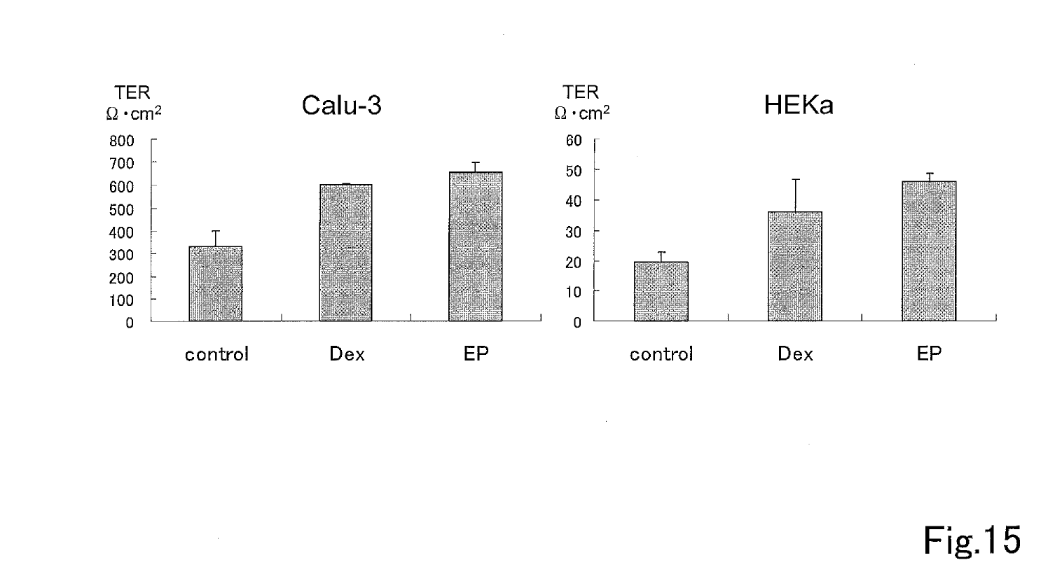

Calu-3 cells were seeded to 3×10⁵ cells/cm² on a cell culture insert (353104, BD Falcon). They were cultured until confluent in 10% FBS RPMI-1640, and once confluence was reached, 0.1% FBS RPMI-1640 containing the following was substituted, and the medium was replaced every day. TER was measured as in Example 11. The results are shown in FIG. 15.
Condition 1: Control
Condition 2: 1 μM dexamethasone
Condition 3: 1 mM EP

(HEKa Human Primary Normal Keratinocytes, Adult)

HEKa cells were seeded to 6×10⁴ cells/cm² on a cell culture insert (353104, BD Falcon). Culture was performed in complete medium consisting of HKGS Kit (S-001-K, Invitrogen) added to Epilife basal medium (M-EPI-500-CA, Invitrogen), and once confluence was reached, a medium containing Calu-3 and the specific compounds listed under Conditions 1 to 3 above was substituted for the complete medium, and medium replacement was performed once a day. TER was measured as in Example 11. The results are shown in FIG. 15.

As shown in FIG. 15, a statistically significant rise in TER value was seen with both Calu-3 and HEKa cells relative to the control (2.0-fold and 2.3-fold, respectively). These results show that the barrier function promoting action of EP is seen generally in epithelial cells. Because EP also promotes cell proliferation and improves TER value in keratinocytes, which make up human skin, it could potentially be used as a powerful aid in preparing high-quality grafts for regenerative medicine, as an additive for treating rough skin or sensitive skin in the cosmetic field, or as an active component of a medicine for treating atopy.

### Example 13

In this example, a comprehensive gene expression analysis of the various actions of EP was performed with a DNA microarray.

(Cells, Array, Reagents, etc.)

Cells of the established normal human airway epithelial cell line BEAS-2B were used in the experiment. These cells were expansively cultured in 10% FBS RPMI-1640, and seeded at a density of 1×10⁵/cm² on a 24-well plate. After 24 hours 0.1% FBS RPMI-1640 was substituted, and EP was administered to a final concentration of 0 or 1 mM (n=4). After 24 hours, the cells were used as the DNA microarray samples. RNA was extracted using a QIAGEN RNeasy Mini Kit. The DNA microarray test was performed using a Whole Human Genome DNA microarray 4×44K (Agilent), and the necessary labeling reagents and the like for the DNA microarray test were all from Agilent.

(Analysis of DNA Microarray Data)

The DNA microarray test data were analyzed statistically with GeneSpring (Agilent) dedicated analysis software, and gene function analysis (Gene Ontology (GO) analysis) was performed based on differential expression. The purpose of the GO analysis is to determine stochastically the degree to which gene sets with statistically significant differential expression correspond to known functional classification gene sets. The calculated p-value represents the probability that a functional classification analogized from differentially expressed genes would occur by chance in the same number of genes selected randomly from the total number of available genes (about 40,000 in the present DNA microarray). The p-value is calculated according to the following formula.

$$f ( x ) = \frac{( \begin{matrix}N p \\ x\end{matrix} ) ( \begin{matrix}Nq \\ n - x\end{matrix} )}{( \begin{matrix}N \\ n\end{matrix} )}$$

N: Total number of genes in array

Np: Total number of genes in array included in a particular functional classification

Nq: Total number of genes not included in a particular functional classification

x: Number of differentially expressed genes with the functional classification

n: Total number of differentially expressed genes

Of the genes with differential expression 24 hours after cell treatment with 1 mM EP, the number of probes* (genes) whose expression was increased to 1.5-fold or more or reduced to 1/1.5 or less (p<0.05) was 801 and 1939, respectively. GO analysis was performed for both, and Tables 7 and 8 show those cases in which 3 or more genes were included in a single functional classification and p<0.05 (top 20 based on p value). Proliferation of BEAS-2B and many other cells was promoted by EP treatment. Of the functions associated with cell proliferation among the genes whose expression was increased by EP treatment, many were gene functions involved in angiogenesis and differentiation (GO: 0001568, GO: 0048514, GO: 0001944). These genes (29 probes*, 20 genes) are examples of functional molecules responsible for the cell proliferation effect of EP (Table 9). Of the genes associated with cell proliferation (GO: 0008283) that did not appear among the top 20 in GO analysis, those that fulfilled the condition of 1.5-fold or more and p<0.05 (16 probes*, 12 genes) are shown in Table 10. This means that these genes may be performance molecules responsible for the cell proliferative function of EP.

A probe is a fragmentary DNA sequence of a gene mounted on a DNA microarray. Because a single gene may have multiple (sometimes overlapping) probes, the number of genes is smaller than the number of probes.

### Example 14

It has already been found that apoptosis (cell self-destruct function) is suppressed by EP. To support this, apoptosis-associated functions are notable among the genes whose expression was reduced (9 of the top 20 functional classifications, Table 8). Gene expression was reduced in the case of 147 probes (123 genes) associated with regulation of apoptosis (GO: 0042981). “Regulation of apoptosis” includes the functions of promoting and suppressing apoptosis. This GO analysis may include some genes that were extracted by the computer based on natural language processing but that are not associated with the actual functions. Thus, the association of the 147 probes with apoptosis was investigated on the PubMed biomedical literature database, with the results shown in Table 16.

TABLE 16

| promotion | suppression | Pro./supp.* | Low Association |
|---|---|---|---|
| 74 | 48 | 3 | 22 |

Although this was not given as an important EP function in GO analysis, promotion or suppression of apoptosis regulator genes whose expression was significantly increased by EP (1.5-fold or more, p<0.05) is shown in Table 17.

TABLE 17

| promotion | suppression | Pro./supp.* | Low Association |
|---|---|---|---|
| 21 | 8 | 0 | 6 |

Apoptosis is suppressed by EP, so of the genes with reduced expression it is the genes with an apoptosis promoting effect that are significant for apoptosis suppression, and conversely out of the genes with increased expression, the genes that suppress apoptosis are examples of performance molecules responsible for the apoptosis suppressing function of EP (Tables 11 to 14).

In particular, one example of an important performance molecule for apoptosis suppression is the FASLG receptor FAS (A_{—}23_P63896, TNF receptor superfamily, member 6) (FAS), transcript variant 1, [NM_{—}000043], a principal cell death ligand expression of which was reduced by 0.36-fold (p=0.0046).

### Example 15

(Comparison of Gene Expression Dynamics in H₂O₂-Treated Samples of Genes, Expression of Which was Reduced by EP)

EP has been identified as a low-molecular-weight substance induced by oxidative stress. It has also been discovered that when administered to cells, EP has an apoptosis suppression action in addition to its cell proliferation function. Because this suggests that EP has some kind of protective function against oxidative stress, a correlational analysis was performed using the typical oxidative stress reagent hydrogen peroxide (H₂O₂).

The data of Example 13 were used as the EP gene expression data. Cell preparation and H₂O₂ administration were performed as in Test 13. Samples that had been treated for 6 hours with H₂O₂ at a final concentration of 125 μM were used as samples for gene expression analysis by DNA microarray.

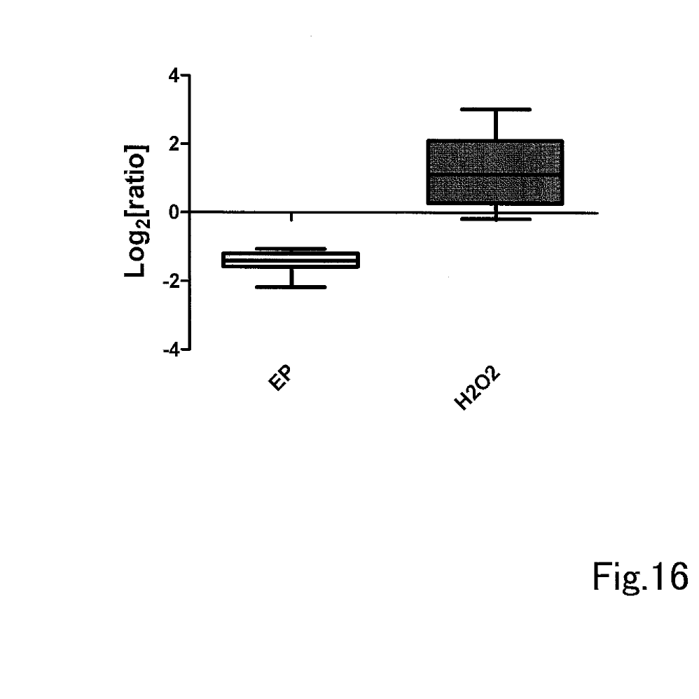

Of the apoptosis-associated genes (apoptosis, GO: 0006915, total 734 probes), expression of which was reduced by 2 times or more (p<0.05) with EP, FIG. 16 shows a boxplot of 30 probes with valid data for which signals could be detected in both EP and H₂O₂ (Present or Marginal in GeneSpring microarray analysis software). The apoptosis-associated genes, expression of which was reduced by EP, generally had their expression increased by the oxidative stress reagent H₂O₂, showing that EP has an action opposite to that of oxidative stress in cells.

(Comparison of Gene Expression Dynamics of all Apoptosis-Associated Genes Between Samples Treated with EP and H₂O₂)

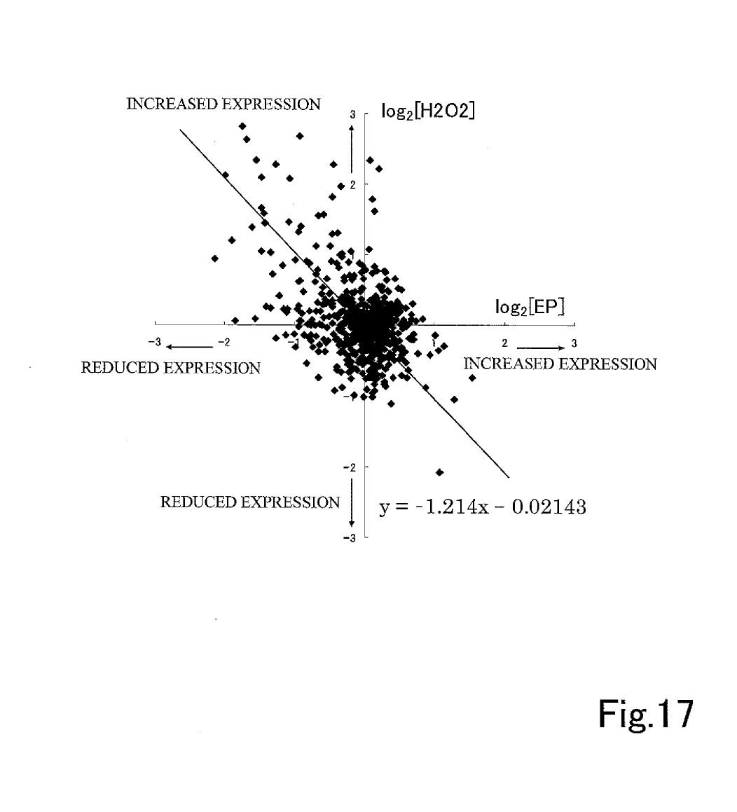

Of all the apoptosis-associated genes (apoptosis, GO: 0006915, total 734 probes), FIG. 17 shows a scatter plot of 611 probes with valid data for which good signals were obtained with EP and H₂O₂. The apoptosis-associated genes with increased and reduced expression exhibited a statistically significant negative correlation at the genome level (Pearson's correlation coefficient 0.48, p=3×10⁻³⁷).

(Correlational Analysis of Gene Expression Dynamics of Oxidation-Reduction Associated Genes Between Samples Treated with EP and H₂O₂).

The scatter plot above shows a negative correlation between the expression dynamics of apoptosis-associated genes treated with EP and H₂O₂. It is thus possible that EP produces a protective (reductive) environment against oxidative stress in cells. Expression of oxidation-reduction associated genes is regulated in response to the oxidation/reduction state in the cell, and assuming that EP has a reductive effect inside the cell, these genes will correlate inversely with dynamics under oxidative stress.

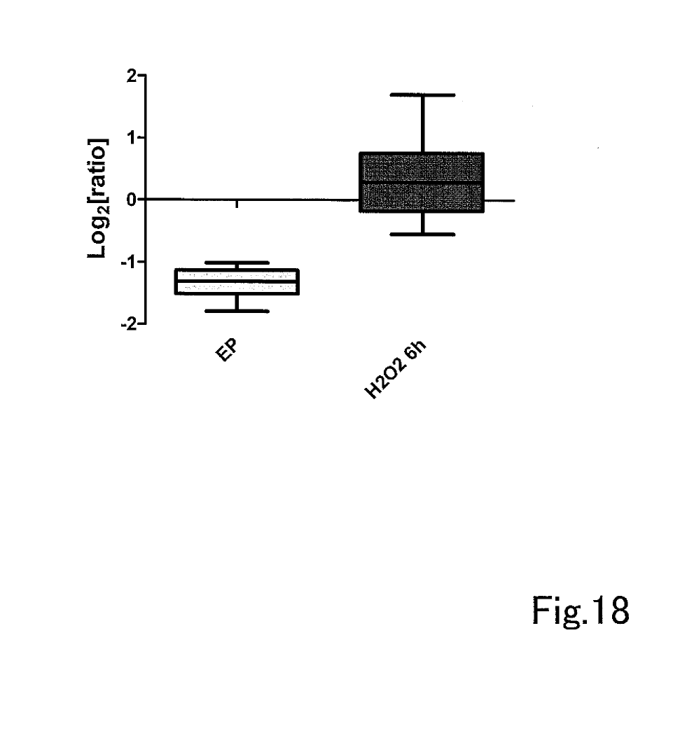

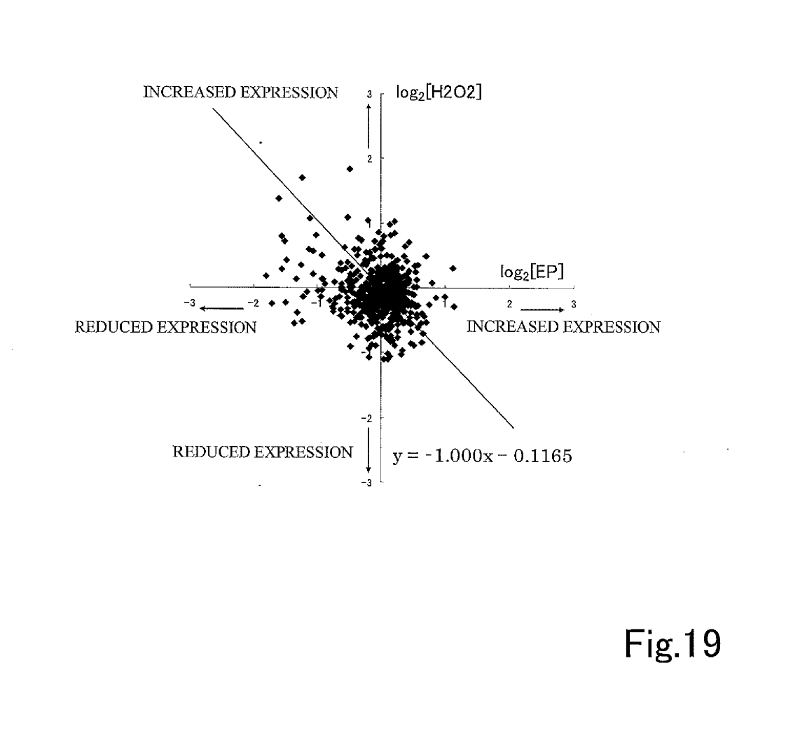

Of the genes associated with oxidation-reduction (GO: 0055114), FIG. 18 and FIG. 19 show those for which a gene expression correlation analysis was performed comparing EP and H₂O₂ (box plot: 21 probes, scatter plot: 614 probes). As with the apoptosis-associated genes, a negative correlation between EP and H₂O₂ treatment of the oxidation-reduction associated genes appeared at the genome level and with the set of genes whose expression was significantly reduced by EP (Pearson's correlation coefficient −0.19, p=2×10⁻⁶).

Focusing on individual genes, there was a 1/2.3-fold decrease (p=0.017) in expression of heme oxygenase-1 (A23_P120883, HMOX1, heme oxygenase (decycling) 1, NM_{—}002133), which is commonly used as a marker of oxidative stress, suggesting a reductive effect of EP in cells. One example of a performance molecule for the anti-oxidative stress function of EP is the gene expression product and antioxidant thioredoxin (A_{—}24_P175519, TXN, thioredoxin, NM_{—}00339), expression of which was increased by 1.4 times (p=0.021) in connection with this reduction effect.

(Comparison of Gene Expression Dynamics of Cytokines and Chemokines with EP and H₂O₂ Treatment)

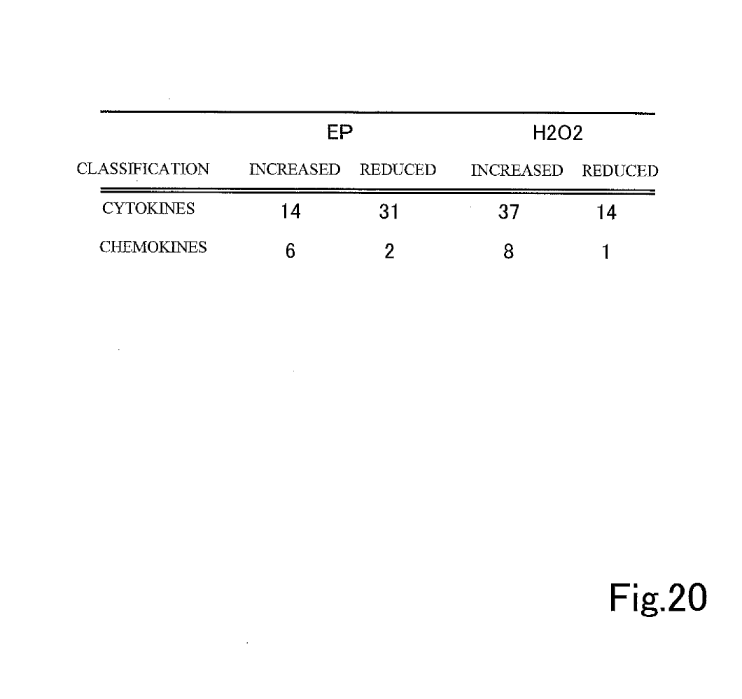

EP has been shown to correlate negatively with the expression dynamics of oxidation-reduction associated genes and with apoptosis caused by oxidative stress. In addition to cell death, oxidative stress is also known to induce expression of cytokines and other genes that induce inflammation, and of chemokines that attract immune cells that cause inflammation. FIG. 20 shows the numbers of genes for which a statistically significant change in expression (expression change of 1.5-fold or more or 1/1.5-fold or less, p<0.05) was produced by EP and H₂O₂ treatment. H₂O₂ treatment induced expression of cytokines and chemokines. EP reduced expression of many cytokines in particularly, and reduction of the cytokines and chemokines shown in Table 15 is an example of its inflammation reaction suppressing function. Of these, examples of inflammation inducing cytokines include AREG, BMP7, CCL27, CSF1, CXCL14, GDF15, IL11, IL18, IL23A, IL31, IL4, IL5, TNFSF15, VEGFA and the like.

Based on these results, since molecules that are altered by EP and the oxidative stress substance H₂O₂ are inversely correlated, EP can be expected to have a protective action (anti-oxidative action) against oxidative stress in cells. To elucidate the mechanism of action of the cell protective effect of EP, we focused on the gene function classifications of oxidation-reduction (GO: 0055114) and cell redox homeostasis (GO: 1145454) associated with anti-oxidation, and extracted data on the differential expression of protective genes against oxidative stress. The results are shown in Table 18.

TABLE 18

| ID | GeneSymbol | Description | GenbankAccession | log2 [ratio] | R* | p-value | PubMed ID |
|---|---|---|---|---|---|---|---|
| A_23_P1361 | ALDH18A1 | h1 | NM_002860 | 0.546 | 1.460 | 0.015 | |
| A_23_P426305 | AOC3 | h2 | NM_003734 | 0.623 | 1.540 | 0.044 | |
| A_23_P151649 | APEX1 | h3 | NM_080649 | 0.541 | 1.455 | 0.010 | 7800476 |
| A_24_P18105 | ASPH | h4 | NM_032466 | 0.642 | 1.560 | 0.018 | |
| A_23_P29754 | BDH1 | h5 | NM_203314 | 0.548 | 1.462 | 0.012 | |
| A_23_P105138 | CAT | h6 | NM_001752 | 0.589 | 1.505 | 0.001 | 16742049 |
| A_23_P40453 | CBR3 | h7 | NM_001236 | 0.491 | 1.406 | 0.006 | 22527884 |
| A_24_P934477 | CBR3 | h8 | AJ000096 | 1.281 | 2.429 | 0.017 | 22527884 |
| A_23_P114662 | CRYZ | h9 | NM_001889 | 0.707 | 1.632 | 0.008 | 19007775 |
| A_24_P914519 | CYBB | h10 | S67289 | 0.968 | 1.956 | 0.012 | |
| A_23_P209625 | CYP1B1 | h11 | NM_000104 | 0.663 | 1.584 | 0.009 | |
| A_23_P58180 | CYP4V2 | h12 | NM_207352 | 1.123 | 2.178 | 0.015 | |
| A_24_P722216 | DHTKD1 | h13 | NM_018706 | 0.515 | 1.429 | 0.013 | |
| A_23_P48740 | DIO2 | h14 | NM_013989 | 0.761 | 1.694 | 0.029 | 20660060 |
| A_23_P122052 | GPX8 | h15 | NM_001008397 | 0.499 | 1.413 | 0.000 | 23201771 |
| A_24_P876522 | GPX8 | h16 | NM_001008397 | 0.680 | 1.602 | 0.016 | 23201771 |
| A_24_P307580 | HTATIP2 | h17 | NM_001098523 | 0.865 | 1.821 | 0.003 | |
| A_23_P64129 | HTATIP2 | h18 | NM_006410 | 0.795 | 1.735 | 0.010 | |
| A_32_P45009 | IDH1 | h19 | NM_005896 | 0.571 | 1.486 | 0.001 | 21506885 |
| A_23_P129313 | IVD | h20 | NM_001159508 | 0.559 | 1.473 | 0.010 | |
| A_23_P127405 | KDM4D | h21 | NM_018039 | 0.534 | 1.448 | 0.007 | |
| A_23_P122216 | LOX | h22 | NM_002317 | 0.711 | 1.637 | 0.001 | |
| A_23_P206661 | NQO1 | h23 | NM_000903 | 0.566 | 1.480 | 0.010 | 8528266 |
| A_23_P326913 | PRUNE2 | h24 | BC019095 | 0.527 | 1.441 | 0.029 | |
| A_23_P258310 | PXDNL | h25 | NM_144651 | 1.278 | 2.426 | 0.019 | |
| A_23_P383278 | PYCRL | h26 | NM_023078 | 0.527 | 1.441 | 0.001 | |
| A_23_P122775 | RTN4IP1 | h27 | NM_032730 | 0.522 | 1.436 | 0.007 | |
| A_23_P132388 | SCO2 | h28 | NM_005138 | 0.580 | 1.495 | 0.002 | |
| A_24_P317708 | SDR16C5 | h29 | NM_138969 | 1.232 | 2.349 | 0.033 | |
| A_32_P39691 | SORD | h30 | NM_003104 | 0.590 | 1.506 | 0.024 | |
| A_23_P90601 | STEAP3 | h31 | NM_182915 | 1.134 | 2.194 | 0.003 | |
| A_24_P200000 | STEAP3 | h32 | NM_182915 | 1.007 | 2.009 | 0.002 | |
| A_23_P138116 | TMEM195 | h33 | NM_001004320 | 0.972 | 1.962 | 0.035 | |
| A_24_P175519 | TXN | h34 | NM_003329 | 0.524 | 1.438 | 0.021 | 22330043 |

| | | |
|---|---|---|
| Description | h1 | Homo sapiens aldehyde dehydrogenase 18 family, member A1 (ALDH18A1), nuclear gene encoding |
| | | mitochondrial protein, transcript variant 1, mRNA [NM_002860] |
| | h2 | Homo sapiens amine oxidase, copper containing 3 (vascular adhesion protein 1) (AOC3), mRNA |
| | h3 | Homo sapiens APEX nuclease (multifunctional DNA repair enzyme) 1 (APEX1), transcript variant 3, mRNA |
| | | [NM_080649] |
| | h4 | Homo sapiens aspartate beta-hydroxylase (ASPH) transcript variant 3, mRNA [NM_0324661 |
| | h5 | Homo sapiens 3-hydroxybutyrate dehydrogenase, type 1 (BDH1), nuclear gene encoding mitochondrial |
| | | protein, transcript variant 3, mRNA [NM_203314] |
| | h6 | Homo sapiens catalase (CAT), mRNA [NM_001752] |
| | h7 | Homo sapiens carbonyl reductase 3 (CBR3), mRNA [NM_001236] |
| | h8 | Homo sapiens mRNA for carbonyl reductase, trapped exon B1084-9, partial [AJ000096] |
| | h9 | Homo sapiens crystallin, zeta (quinone reductase) (CRYZ), transcript variant 2, mRNA [NM_001889] |
| | h10 | CYBB = cytochrome b558 gp91-phox subunit [human mRNA Partial Mutant, 102 nt]. [S67289] |
| | h11 | Homo sapiens cytochrome P450, family 1, subfamily B, polypeptide 1 (CYP1B1), mRNA [NM_000104] |
| | h12 | Homo sapiens cytochrome P450, family 4, subfamily V, polypeptide 2 (CYP4V2), mRNA [NM_207352] |
| | h13 | Homo sapiens dehydrogenase E1 and transketolase domain containing 1 (DHTKD1), nuclear gene encoding |
| | | mitochondrial protein, mRNA [NM_018706] |
| | h14 | Homo sapiens deiodinase, iodothyronine, type II (DIO2), transcript variant 1, mRNA [NM_012989] |
| | h15 | Homo sapiens glutathione peroxidase 8 (putative) (GPX8), mRNA [NM_001008397] |
| | h16 | Homo sapiens glutathione peroxidase 8 (putative) (GPX8), mRNA [NM_001008397] |
| | h17 | Homo sapiens HIV-1 Tat interactive protein 2, 30 kDa (HTATIP2), transcript variant 5, mRNA |
| | h18 | Homo sapiens HIV-1 Tat interactive protein 2, 30 kDa (HTATIP2), transcript variant 2 mRNA [NM_006410] |
| | h19 | Homo sapiens isocitrate dehydrogenase 1 (NADP+) soluble (IDH1), mRNA [NM_005896] |
| | h20 | Homo sapiens isovaleryl Coenzyme A dehydrogenase (IVD), transcript variant 2, mRNA [NM_001159508] |
| | h21 | Homo sapiens lysine (K)-specific demethylase 4D (KDM40), mRNA [NM_018039] |
| | h22 | Homo sapiens lysyl oxidase (LOX), mRNA [NM_002317] |
| | h23 | Homo sapiens NAD(P)H dehydrogenase, quinone 1 (NQO1), transcript variant 1, mRNA [NM_000903] |
| | h24 | Homo sapiens prune homolog 2 (Drosophila), mRNA (oDNA clone IMAGE: 5015761), [BC019095] |
| | h25 | Homo sapiens peroxidasin homolog (Drosophila)-like (PXDNL), mRNA [NM_144651] |
| | h26 | Homo sapiens pyrroline-5-carboxylate reductase-like (PYCRL), mRNA [NM_023078] |
| | h27 | Homo sapiens raticulon 4 interacting protein 1 (RTN4IP1), nuclear gene encoding mitochondrial protein, |
| | | mRNA [NM_032730] |
| | h28 | Homo sapiens SCO cytochrome oxidase deficient homolog 2 (yeast) (SCO2), nuclear gene encoding |
| | | mitochondrial protein, transcript variant 1, mRNA [NM_005138] |
| | h29 | Homo sapiens short chain dehydrogenase/reductase family 16C, member 5 (SDR16C5), mRNA [NM_138969] |
| | h30 | Homo sapiens sorbitol dehydrogenase (SORD), mRNA [NM_003104] |
| | h31 | Homo sapiens STEAP family member 3 (STEAP3) transcript variant 1, mRNA [NM_182915] |
| | h32 | Homo sapiens STEAP family member 3 (STEAP3), transcript variant 1, mRNA [NM_182915] |
| | h33 | Homo sapiens transmembrane protein 195 (TMEM195) mRNA [NM_001004320] |
| | h34 | Homo sapiens thioredoxin (TXN), mRNA [NM_003329] |

R*: Ratio to control (times)

Of the genes associated with oxidation-reduction (GO: 0055114) and cell redox homeostasis (GO: 0045454), 34 probes fulfilled the conditions of a 1.4-fold or greater increase in expression due to EP and p<0.05 or less as shown in Table 18. It is thought that in some of these cases, increased gene expression may have a cell protective and anti-oxidative effect, or the effect of protecting tissue against diseases caused by oxidative stress. The biomedical literature database PubMed was searched for genes that cause oxidative stress or disease due to oxidative stress when their expression is reduced or their function is lost, and the PubMed IDs for those for which an association was found are shown in the rightmost column of Table 18.

In the gene sets corresponding to oxidation-reduction (GO: 0055114) and cell redox homeostasis (GO: 0045454), in addition to TXN as discussed above, EP causes increased expression of catalase (A_{—}23_P105138, CAT, catalase (CAT), mRNA [NM_{—}001752], NM_{—}001752) and NADPH:quinone reductase-1 (A_{—}23 P206661, NQ01, NAD(P)H dehydrogenase, quinone 1 (NQO1), transcript variant 1, mRNA, NM_{—}000903) and other active oxygen-scavenging enzymes and detoxification metabolism enzymes. In particular, because CAT functions as a hydrogen peroxide scavenger, increased expression of this gene has a protective function against cell damage caused by active oxygen. NQO1 is an enzyme associated with reduction of quinones, and is known as an antioxidant enzyme that suppresses production of active oxygen.

### Example 16

(Confirmation of Oxidative Stress Resistance Resulting from EP Treatment)

Gene expression changes caused by EP correlate inversely with changes caused by oxidative stress. In particular, reduced expression of the oxidative stress marker HMOX1 suggests that a reductive environment rather than a static state has been produced inside the cell, and this is supported by increased expression of the antioxidant TXN. The above is a functional analogy based on changes in gene expression, and it is still necessary to show that EP actually reduces cell toxicity caused by oxidative stress. The following test was performed for this reason.

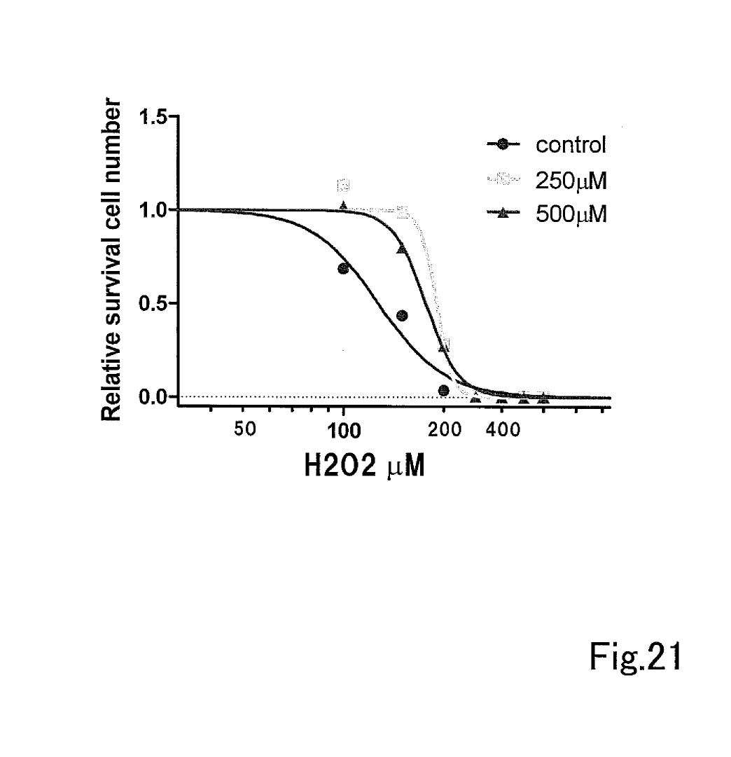

Cells of the normal airway epithelial cell line BEAS-2B were seeded at a density of 1×10⁵/cm² on a 96-well plate, and after 24 hours the medium was replaced with 0.5% FBS RPMI_{—}1640 containing EP at final concentrations of 0, 250 and 500 μM. 24 hours after medium replacement the medium was replaced with 0.5% FBS RPMI-1640, and serially diluted H₂O₂ was administered (n=4). 24 hours after H₂O₂ administration a cell toxicity test WST-8 assay (Cell Counting Kit-8, Dojindo) was performed. For the data analysis, the 50% cell death concentration EC₅₀ was calculated with GraphPad Prism 5 statistical analysis software. FIG. 21 shows a dose-dependency curve of cell death caused by H₂O₂ in cells pre-treated with 0, 250 μM and 500 μM of EP.

As shown in FIG. 21, pre-treatment with 250 μM and 500 μM of EP caused toxicity to appear at higher concentrations of H₂O₂ than in the group without EP treatment, indicating that the cells had acquired resistance to oxidative stress. In general, the cytotoxicity of a compound is proportional to the inverse of the 50% cell death concentration EC₅₀. The EC₅₀ in each EP pre-treatment group and the relative toxicity in each case are shown in Table 19.

TABLE 19

| | control | 250 uM | 500 uM |
|---|---|---|---|
| EC_{50 (uM)} | 127.7 | 190.4 | 177.3 |
| 1/EC₅₀ | 0.00783 | 0.00525 | 0.00564 |
| Relative toxity* | 1.00 | 0.67 | 0.72 |
| Toxity | — | 33 | 28 |
| reduction rate % | | | |

As shown in Table 19, the cytotoxicity of H₂O₂ was reduced by about 30% by pre-treatment with 250 to 500 μM of EP. This means that in addition to gene expression changes at the genome level, EP imparts resistance to oxidative stress as a cell phenotype.

### Example 17

Example 15 showed clearly that EP suppresses expression of many cytokines and chemokines. This suggests that EP may have an anti-inflammatory action. Inflammation is an acute reaction that may occur in any tissue due to stimulus by bacteria and other foreign bodies, and leads to increased vascular permeability, infiltration of lymphocytes and other immunocompetent cells into the inflammation site, and an expanded inflammation reaction caused by secretion of inflammation-inducing cytokines by the immunocompetent cells. The cells used in Example 15 are airway epithelial cells, and the same effects need to be confirmed in immunocompetent cells. Moreover, Example 15 shows effects at the gene expression level, and to show an anti-inflammatory effect it is necessary to show secretion suppression at the protein level, which is the functional level of the inflammation-inducing cytokines and chemokines secreted by the inflammatory cells. In the examples below, the anti-inflammatory action of EP was confirmed by the methods of Singh et al. (Clin. Chem., 2005 December; 51(12): 2252-6).

Human monocytic cultured THP-1 cells were cultured logarithmically as follows in 10% FBS RPMI-1640 growth culture. Cells were seeded to 4×10⁵/ml on a 24-well plate (500 μl/well), additional growth culture containing EP was added, and finally the EP concentration was adjusted to the target concentration (0.1 mM, 1 mM). 2 hours after EP administration, further growth medium containing lipopolysaccharides (LPS) was added, and adjusted to concentrations of 0.1 μg/ml and 1 μg/ml.

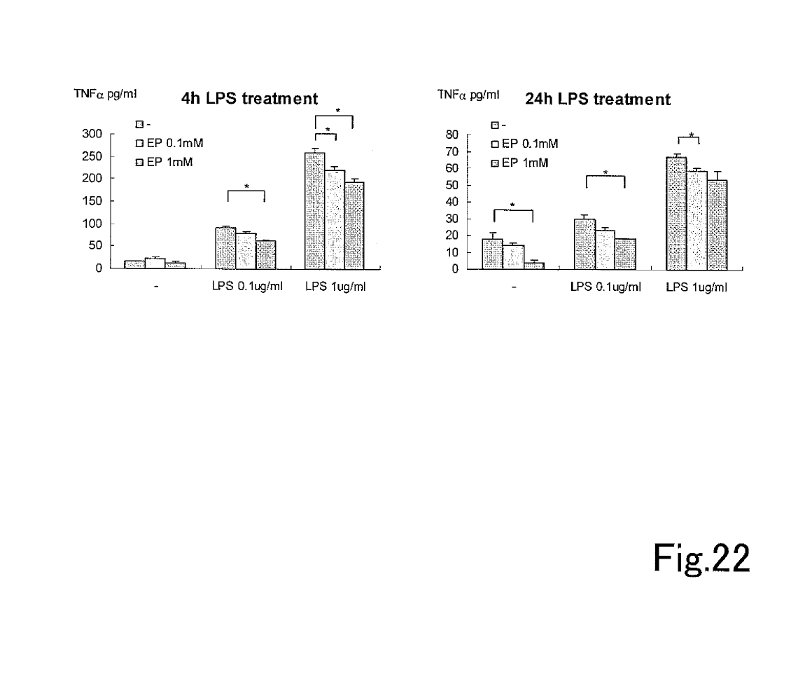

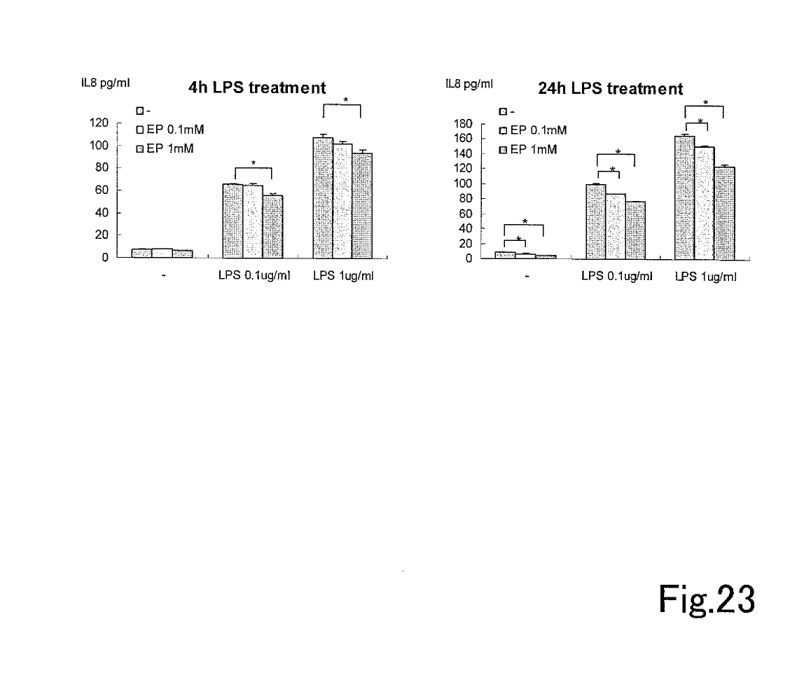

4 hours and 24 hours after lipopolysaccharide administration, the medium was collected, the cells were precipitated by centrifugation, and the supernatant was used as an ELISA sample. As in the Singh et al. report, the inflammatory cytokine TNF-α and the chemokine IL-8, which attracts inflammatory cells, were used as ELISA measurement indicators. The results are shown in FIG. 22 and FIG. 23.

Singh et al. report that anti-inflammatory drugs can be screened by suppressing TNF-α secretion caused by short-term LPS treatment (cited above). This is because the promotion of TNF-α secretion by LPS stimulus normally peaks within about 2 to 6 hours. As shown in FIG. 22, while the amount of TNF-α induced by LPS stimulus (1 μg/ml) was about 260 pg/ml (without EP) with 4 hours of stimulus, it fell to about 70 pg/ml (EP 1 mM) with 24 hours of stimulus, a result that matches the evaluation of Singh et al. Moreover, as shown in FIG. 22, EP dose-dependently suppressed secretion of induced TNF-α after both 4 hours and 24 hours of stimulus.

As shown in FIG. 23, moreover, EP also dose-dependently suppressed secretion of IL-8. IL-8 secretion in response to LPS stimulus tended to be greater after 24 hours than after 4 hours, but EP had an obvious IL-8 secretion suppression effect even after 24 hours of stimulus.

Moreover, in FIGS. 22 and 23, after 24 hours (26^{th} hour of EP treatment counting 2 hours of pre-treatment), EP suppressed secretion of TNF-α and IL-8 even in cells not treated with LPS. That is, TNF-α secretion was reduced to about 78% and IL-8 secretion to about 43% by 26 hours of treatment with 1 mM EP. This shows that even without any special LPS or other stimulus, there is a constant secretion suppressing effect on cytokines and chemokines produced by macrophage and other immunocompetent cells.

In recent years, there have been reports that concentrations of TNF-α and other inflammatory cytokines in the blood may be increased due to obesity even in the absence of any particular injury or infection, as in the case of metabolic syndrome (Isr Med Assoc J. 2008 July; 10(7): 494-8). It is also thought that TNF-α is produced by macrophages when oxidized LDL increases in the blood of obese patients, promoting arteriosclerosis (Artrioscler Thromb Vase Biol. 1996 December; 16(12); 1573-9).

For these reasons, EP is not only expected to have anti-inflammatory effects, but may have preventative and therapeutic effects against diseases caused by cytokines and chemokines induced by inflammation (arteriosclerosis or myocardial infarction, cerebral infarction and other obstructive vascular insults caused by arteriosclerosis).

Specific examples of the present invention have been described in detail, however, these are mere exemplary indications and thus do not limit the scope of the claims. The art described in the claims includes modifications and variations of the specific examples presented above.

Technical features described in the description and the drawings may technically be useful alone or in various combinations, and are not limited to the combinations as originally claimed. Further, the art described in the description and the drawings may concurrently achieve a plurality of aims, and technical significance thereof resides in achieving any one of such aims.

1. A method of inhibiting oxidative stress to a cell, comprising contacting the cell with an effective amount of ethanolamine phosphate.

2. The method according to claim 1, wherein the cell is an animal cell.

3. The method according to claim 2, wherein the animal cell is a mammalian cell.

4. The method according to claim 1, wherein the cell is contacted with ethanolamine phosphate in vitro.

5. The method according to claim 1, wherein the cell is contacted with ethanolamine phosphate in vivo.

6. The method according to claim 1, wherein the ethanolamine phosphate is administered to a living body.

7. The method according to claim 6, wherein the living body is an animal.

8. The method according to claim 7, wherein the animal is a human.

9. A method for preventing or treating a disease or a condition caused by oxidative stress or oxidation, comprising the method of claim 7.

10. A food composition comprising ethanolamine phosphate as an anti-oxidative agent.

11. A nutritional composition comprising ethanolamine phosphate as an anti-oxidative agent.
